(19) United States
(12) Patent Application Publication
(10) Pub. No.: US 2003/0134420 A1
(43) Pub. Date: Jul. 17, 2003

(54) Methods and compositions for gene delivery

(76) Inventors: Charles Peter Lollo, Encinitas, CA (US); Mariusz Banaszczyk, San Marcos, CA (US); Henry C. Chiou, Encinitas, CA (US); Dongpei Wu, San Diego, CA (US); Patricia M. Mullein, Encinitas, CA (US); Alison T. Carlo, Rancho Santa Fe, CA (US)

Correspondence Address: LAHIVE & COCKFIELD, 28 STATE STREET, BOSTON, MA, 02109, US

(21) Appl. No.: 10/211,214

(22) Filed: Aug. 2, 2002

Related U.S. Application Data
(63) Continuation of application No. PCT/US01/05234, filed on Feb. 16, 2001.
(60) Provisional application No. 60/183,516, filed on Feb. 18, 2000.

(51) Int. Cl. A61K 38/00; C12N 15/85

(52) U.S. Cl. 435/455; 514/12

(57) ABSTRACT

The present invention provides novel compositions and formulations for delivering anionic compounds, particularly polynucleotides (DNA and RNA), across cellular boundaries (e.g., cellular membranes) either in vivo or in vitro.

### RELATED APPLICATIONS

This application claims priority to U.S. Provisional Patent Application Serial No. 60/183,516, filed on Feb. 18, 2000, and PCT Application No. PCT/US01/05234 filed Feb. 16, 2001, published in English in accordance with PCT Article 21(2), the contents of each of which are hereby incorporated herein by reference.

### BACKGROUND OF THE INVENTION

Gene therapy is making steady progress through preclinical development. Nonviral gene delivery systems currently under development are naked DNA, cationic liposomes, cationic polymers, and combinations of both; cationic lipids with cationic polymers (Hickman, et al. Hum Gene Ther 1994, 5, 1477-1483; Wolff, J. A.; et al. Science 1990, 247, 1465-1468; Felgner, P. L. et al. Hum Gene Ther 1997, 8, 511-512; Felgner, et al. Ann NY Acad Sci 1995, 772, 126-139; Mahato, R. I. et al Pharm Res 1997, 14, 853-859; Nicolau, C. et al. Crit Rev Ther Drug Carrier Syst 1989, 6, 239-271; Lasic, et al. J Am Chem Soc 1997, 119, 832-833; Wolfert, et al. Hum Gene Ther 1996, 7, 2123-2133; Tang et al. Gene Ther 1997, 4, 823-832; Haensler, J. et al. Bioconjug Chem. 1993, 4, 372-379; Kabanov et al. Bioconjug Chem 1995, 6, 7-20; Gottschalk et al. Gene Ther 1996, 3, 48-57; Wu, et al. J Biol Chem 1988, 263, 14621-14624; Kwok, et al. Journal of Pharmaceutical Sciences 1999, 88(10), 996; Katayose et al. Bioconjugate Chem. 1997, 8, 702; Lee et al. Hum Gene Ther 1996, 7, 1701-1717). In general, these delivery systems suffer from, simply termed, “serum effects” and show low levels of gene expression in vivo (Lollo, et al. Blood Coagulation and Fibrinolysis 1997, 8, S31-S38). For example, DNA poly-L-lysine complexes (polyplexes) are cleared quickly from the vascular compartment and extensive DNA degradation is detected (Ley, et al. 1998, Keystone Symposia, Colorado, ORGN Abstract #4106). These polyplexes show two additional shortcomings. One is colloidal instability, which has been recently alleviated by PEGylation (Hansma et al. Nucleic Acids Research 1998, 26, 2481-2487; Lee, et al. J Biol Chem 1996,271, 8481-8487; Banaszczyk, et al. J.M.S. -Pure Appl. Chem. 1999, A36(7&8), 1061; Dash et al. Gene Therapy 1999, 6, 643; Ogris, et al. Gene Therapy 1999, 6, 595; Wolfert et al. Human Gene Therapy 1996, 7, 2123; Kwok et al. J. Pharm. Sci. 1999, 88(10), 996). The second is instability of polyplexes when exposed to anionic molecules in vitro and in vivo (Ruponen, et al. Biochim. Biophys. Acta 1999, 1415, 331).

In general, complexes of DNA with either cationic lipids or cationic polymers must protect DNA from degradation in extracellular (vascular) compartment, and advantageously should remain intact. However, both cationic lipid and cationic polymer DNA complexes, when challenged with negatively charged molecules (such as those which are typically present in extracellular space) will, to a varying extent, release DNA. These complexes are, generally, unstable and labile. The premature DNA release from labile complexes can result in rapid DNA degradation and poor transfection efficiency. The complex lability and colloidal instability is a challenge for designers of effective gene delivery methods and compositions.

### SUMMARY OF THE INVENTION

The present invention provides novel compositions and formulations for delivering anionic compounds, particularly polynucleotides (DNA and RNA), across cellular boundaries (e.g., cellular membranes) either in vivo or in vitro.

In one embodiment, the invention provides novel molecular complexes, referred to as “polyplexes,” containing an anionic compound, such as a nucleic acid, associated with one or, more typically, multiple co-polymer domains, including a cationic domain, a transitional domain, and/or a surface domain. The co-polymer domains function as “delivery enhancers” to facilitate delivery of the anionic compound across cellular boundaries by interacting with or “encapsulating” the anionic compound. The surface domain of the polyplexes optionally also can include cellular ligands which target polyplexes to cells.

In another embodiment, the invention provides formulants or “penetration enhancers” which can be combined with polyplexes of the invention, or with free (“naked”) nucleic acids, to further enhance the ability of these compositions to traverse cellular membranes (i.e., be taken up by cells). Suitable penetration enhancers include, for example, DHPC, bile salts, surfactants and combinations thereof. Other techniques, such as sonification, also can be used in conjunction with the present invention to enhance cellular uptake of polyplexes.

Polyplex compositions and formulations of the present invention can be used to enhance delivery and uptake of a wide variety of therapeutic agents agents by cells, particularly in applications of gene therapy.

In an embodiment, the invention pertains, at least in part, to a method of delivering an anionic agent through a lipid membrane. The method includes contacting the anionic agent with a delivery enhancing formulation, allowing a polyplex to form; and contacting the lipid membrane with a penetration enhancer, such that upon contact of the polyplex with the lipid membrane, the anionic agent is delivered through the membrane. The delivery enhancing formulation contains one or more components selected from a cationic backbone moiety, a hydrophobic moiety, and a hydrophilic moiety. Typically, the formulation contains all three components.

In another embodiment, the invention pertains to a method for enhancing expression of a nucleic acid in a cell. The method includes contacting the nucleic acid with a delivery enhancing formulation (as described above), allowing a polyplex to form, and contacting the membrane of the cell with a penetration enhancer, such that upon contact of the polyplex with the membrane of the cell, the nucleic acid is internalized into the cell and expression of said nucleic acid is enhanced.

In yet another embodiment, the invention pertains to a method for treating a subject by administering an effective amount of a penetration enhancer and a polyplex of the present invention (e.g., comprising a nucleic acid, a cationic backbone moiety, a hydrophobic moiety, and a hydrophilic moiety), such that said subject is treated. The penetration enhancer can be administered before, after or concurrently with the polyplex.

The invention also pertains, at least in part, to polyplexes of the invention, comprising copolymers as described herein and anionic agents (e.g., nucleic acids, etc.). The invention also pertains to pharmaceutical compositions comprising such polyplexes along with an effective amount of a penetration enhancer, combined in a pharmaceutically acceptable carrier to form a therapeutic composition.

In yet another embodiment, the invention to pertains to a method for enhancing expression of a nucleic acid in a cell by contacting the cell with a free nucleic acid (i.e., not in the form of a polyplex) and a penetration enhancer, such that the expression of the nucleic acid is enhanced.

### BRIEF DESCRIPTION OF THE FIGURES

FIG. 1 shows a polynucleotide carrier complex in Cartesian coordinates.

FIG. 2 is a drawing of the polynucleotide carrier complex of FIG. 1.

FIG. 3 shows the interaction between a ligand on the surface domain of a polyplex interacting with a cellular receptor.

FIG. 4 shows polyplex and lipid membrane in equilibrium with a formulant.

FIG. 5 shows polyplex containing polymers comprising two hydrophobic domains linked with hydrophilic polymer. FIG. 5A depicts polyplex with polymers with one hydrophobic domain each. FIG. 5B depicts polyplex formed with conjugates containing two hydrophobic domains, one of which is shown in its unbound state. FIG. 5C shows polyplex with conjugates with two hydrophobic domains in the bound state.

FIG. 6 shows polyplex fusion with cellular membrane facilitated by residues in the second hydrophobic domain of the conjugates.

FIG. 7 is a representation of n-block co-polymer.

FIG. 8 is a representation of the compounds described in Table 1.

FIG. 9 shows the structure of (a) randomly grafted hydrophilic PEG chains and randomly grafted hydrophobic chains on a cationic domain and (b) randomly grafted hydrophobic-hydrophilic element on a cationic domain.

FIG. 10 shows the structure of grafted polymers with one hydrophobic domain per PEG chain. FIG. 10a shows a hydrophobic domain between a cationic domain and hydrophilic domains. FIG. 10b shows a hydrophobic domain positioned at the terminus of hydrophilic domain that is then grafted on a cationic domain.

FIG. 11 shows the structure of grafted polymers with two hydrophobic domains per PEG chain. FIG. 11a shows a hydrophobic domain between the cationic domain and the surface domain. FIG. 11b shows a hydrophobic domain positioned at the terminus of a surface (e.g., hydrophilic) domain, and between the surface (e.g., hydrophilic) and cationic domains.

FIG. 12 shows the equilibrium between the polynucleotide carrier complex (B) with unincorporated formulant (A) and the polynucleotide carrier complex with incorporated formulant (C).

FIG. 13 is a bar graph showing the effect of a penetration enhancer on the expression of luciferase encapsulated in a polyplex of the invention. From the left, the first bar of the graph (white) represents a polyplex formed from copolymers comprised of random grafts of PEG5k on PLL10k chain, the second bar (white) represents a polyplex comprised of copolymers of the formula PLL10k-g-(ε-NH-PEG5k)_{14.3}. The remaining bars on the graph represent polyplexes comprised of PLL10k-g-(ε-NH—C₁₀-O-PEG2k)₉ with different penetration enhancing formulants. The third bar (grey) represents no additional formulation enhancer. The fourth bar (white) represents the result with added 0.19% Brij 35 formulant. The fifth bar (black) represents the result with added 0.41% OGP formulant. The sixth bar (horizontal lines) represents the result with added 0.5% TCDC formulant. The seventh bar (diagonal lines) represents the results with added 0.4% DHPC formulant.

FIG. 14 is a graph showing the effects of polyplexes comprised of different copolymers on luciferase expression when administer with the formulant, DHPC. ‘●’ represents PLL9.4k-g-(ε-NH-“Chenodeoxycholic Acid”)₁₆, ‘▴’ represents the copolymer PLL9.4k-g-(ε-NH-“Cholic Acid”)₁₀, ‘▾’ represents the copolymer PEG5k-b-(Cys-S—C18)₁₀-b-(Lys)₄₅-g-(ε-NH-Chenodeoxycholic Acid)₁₀, ‘♦’ represents the copolymer PEG5k-g-(Cys-S—C18)₁₀-b-(Lys)₁₂₀-g-(β-NH-“Chenodeoxycholic Acid”)₁₅, ‘♦’ represents the copolymer PLL9.4k-g-(ε-NH-“Chenodeoxycholic Acid”)₁₆, and ‘□’ represents the copolymer PLL9.4k-g-(ε-NH—C₁₀-PEG2k)₁₄.

FIG. 15 is a graph showing the effects of the addition of the formulant DHPC on expression of luciferase, when administered with polyplex of the invention. In FIG. 15, the symbol ‘●’ represents a polyplex formed from the copolymer, PLL9.4k-g-(ε-NH—CO-“Trigalactose”)_{16.1}, the symbol ‘▴’ represents a polyplex formed from the copolymer, PLL9.4k-g-(ε-NH—C12-PEG5k)_{4.7}-g-(ε-NH-“Trigalactose”)₉), the symbol ‘▾’ represents a polyplex formed from the copolymer, PLL9.4k-g-(ε-NH—CO-“Trigalactose”)_{16.1} coadministered with DHPC, and the symbol ‘♦’ represents a polyplex formed from the copolymer, PLL9.4k-g-(ε-NH—C 12-PEG5k)_{4.7}-g-(ε-NH-“Trigalactose”)₉) coadministered with DHPC

FIG. 16 is a graph showing the expression of luciferase in mice when the gene is administered with a variety of co-polymer polyplexes that were formulated with DHPC. The symbol ‘●’ represents a polyplex which were formed using copolymers constructed from random grafts of hydrophobe (—CH₂CONHCH₂CH₂CH₂—O-β-Cholesterol ether) and PEG, PLL9.4k-g-(ε-NH-PEG5k)_{12.8}-g-(ε-NH—CH₂CONHCH₂CH₂CH₂—O-β-Cholesterol ether)₂₆. The symbol ‘▴’ represents polyplexes comprised of the block co-polymer (PEG5k-b-(Cys-S—C18)₁₀-b-(Lys)₄₅); and the symbol ‘♦’ represents polyplexes comprised of the block co-polymer (PEG5k-b-(Phe)₁₄-b-(Lys)₅₁). Polyplexes comprised of polymers consisting of random grafts of PEG-coupled-hydrophobe with and without Trigalactose ligand, include PLL9.4k-g-(ε-NH-PEG4.4k-C18)_{2.8} represented by the symbol ‘♦’, PLL10k-g-(ε-NH—C₁₀-PEG4.4k-C18)_{6.6} represented by the symbol ‘♦’, PLL9.4k-g-(ε-NH—C₁₂-PEG5k)_{4.7}-g-(ε-NH—CH2CO-“Trigal”)₉ represented by the symbol ‘□’.

FIGS. 17A and 17B are bar graphs which show the biodistribution of ¹²⁵I-pCMVβGal when free (light grey), free with TCDC (medium light grey), encapsulated in a polyplex comprised of block co-polymer (PEG5k-b-(Cys-S—C18)₁₀-b-(Lys)₄₅) (BP-A) (dark grey) and encapsulated in a polyplex comprised of block co-polymer (PEG5k-b-(Cys-S—C18)₁₀-b-(Lys)₄₅) (BP-A) with TCDC (black). The biodistribution is determined at 5 minutes (FIG. 17A) and one hour (FIG. 17B).

### DETAILED DESCRIPTION OF THE INVENTION

I Polyplexes

The present invention provides, in one aspect, molecular complexes referred to as “polyplexes” for delivering anionic agents (e.g., anionic polymers or negatively charged therapeutic agents, such as DNA, RNA, proteins, and small molecules) through lipid membranes (e.g., cellular boundaries, e.g., cellular membranes, nuclear membranes, endosomal membranes, etc.). The complexes are referred to as “polyplexes” because the multiple components, which make up the complexes, interact through both covalent and non-covalent bonds.

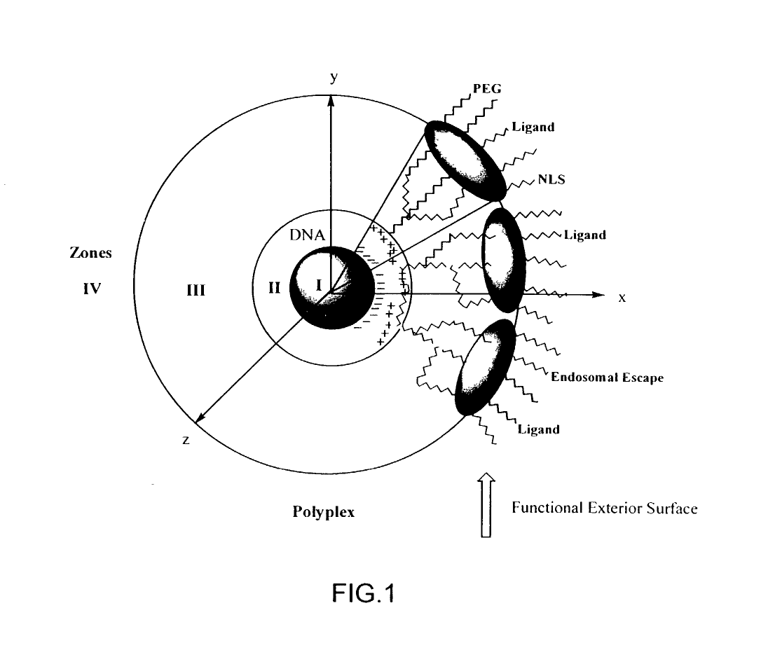

As shown in FIG. 1, polyplexes of the present invention are made up of multiple co-polymer domains. These domains are organized by the type of functional groups present on the co-polymer making up the domain. Typically, the center domain (Zone I of FIG. 1) contains the anionic agent. Examples of anionic agents include nucleic acids, negatively charged drugs and other small molecules capable of being delivered via a polyplex through a cellular boundary or lipid membrane. The cationic domain (Zone II of FIG. 1) is designed to interact, e.g., electrostatically, with the anionic domain/agent. Generally, the cationic domain is comprised of one or more cationic backbone moieties of copolymers, which are described in greater detail below. The transitional domain (Zone III of FIG. 1) links the cationic domain with the surface domain, typically via linear or branched co-polymers. The transitional domain may be hydrophobic in nature and may be comprised, at least in part, of hydrophobic moieties of copolymers. When the transitional domain is comprised at least in part of hydrophobic moieties, it is generally referred to as the “hydrophobic domain.” Finally, the surface domain (Zone IV of FIG. 1) defines the polyplex surface by way of, for example, branching elements which allow the introduction of multiple molecules or other polymers on the polyplex surface. Such moieties modify the surface properties of the polyplex so as to enhance overall delivery of the anionic agent. The surface domain may be comprised, at least in part, of hydrophilic moieties of copolymers, as well as other ligands and other surface moieties which allow the polyplex to perform its intended function.

Overall, polyplexes of the invention essentially consist of multiple co-polymer domains which interact (e.g., as a carrier) with an anionic agent which is delivered across a cell boundary or lipid membrane.

In order to minimize steric hindrance, the functional moieties of the polyplexes can first be attached to a single grafting element which, in turn, can then be grafted onto a desired cationic domain. For example, in one embodiment, a hydrophobic moiety is coupled to PEG (a hydrophilic moiety) and then grafted on to a cationic domain.

Delivery of anionic agents to cells or cellular compartments using polyplexes of the invention can, in certain embodiments, be further enhanced using ligand-receptor interactions, endosome disruptive residues, and nuclear localizing sequences. These surface moieties may also aid in polyplex delivery by protecting the polyplex from deleterious interactions in, for example, vascular compartments. Further enhancement can be achieved by attaching additional hydrophobic moieties to the cationic, transition and/or surface domains, such as lather releasing molecules that change permeability of membrane barriers, and as a result, increase overall uptake and expression. Furthermore, other pentration enhancers can also be used to enhance the permeability of the membrane barriers.

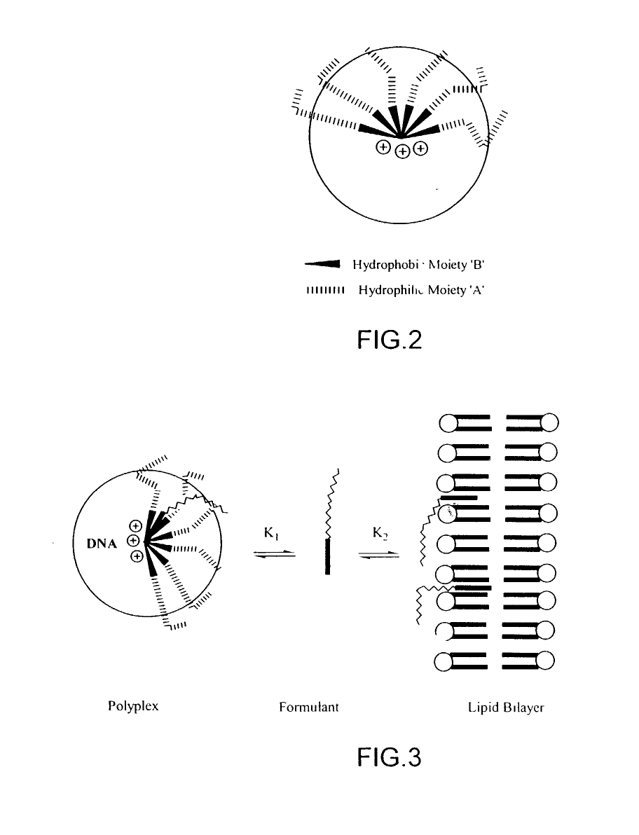

A simplified representation of a polyplex made up of co-polymers with one hydrophobic moiety each (i.e., one per co polymer) is shown in FIG. 2. Many additional features present in the zones or domains of the polyplex are omitted for clarity, and only interior and exterior residues are show.

Polyplexes of the invention can be formed, in one embodiment, with polymers containing one hydrophobic moiety on a grafted cationic backbone moiety. The hydrophobic moiety aids with DNA condensation as evidenced by fluorescent quenching assay. Additional hydrophobic moieties grafted on to the cationic backbone moiety can be used to increase the hydrophobicity of the polyplex. The hydrophobic moieties, through the process of self association, micellization-like processes, and co-micellization processes, can interact with formulant or penetration enhancer molecules which may enhance delivery of the anionic agent through the lipid membrane.

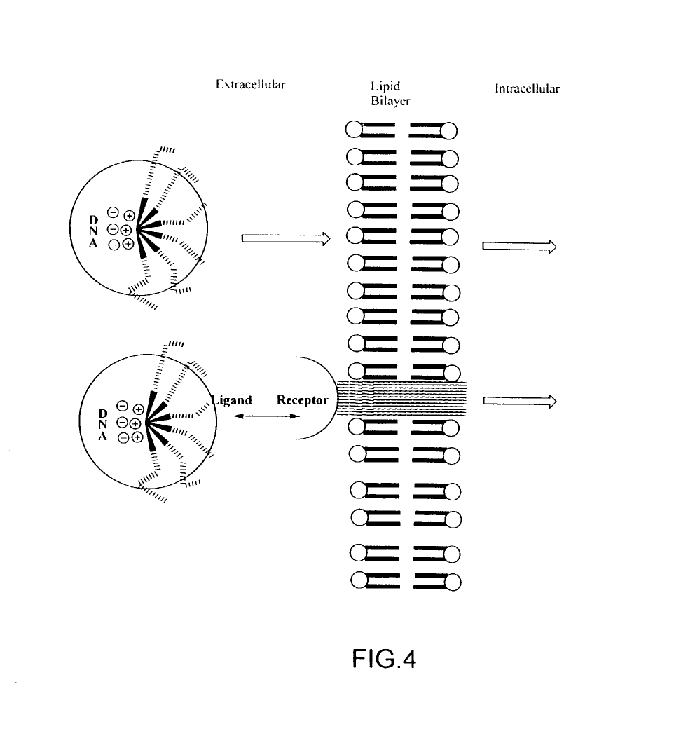

In addition to including cellular ligands, nuclear ligands, endosomal escape mechanisms and other delivery (e.g., targeting) agents, polyplexes of the invention can be formulated with permeation enhancers and other delivery formulants which are coadministered with the polyplex. Such delivery formulants of the invention also can be used to enhance delivery of free DNA. Both ligand-receptor mediated (specific) and nonspecific modes of cellular entry are shown in FIG. 3. FIG. 4 shows that specific cellular entry (e.g., via ligand interactions) can be further enhanced by the coadministration of permeation enhancers. Suitable cellular ligands for incorporation into polyplexes of the invention can include, for example, any natural or synthetic ligand which is capable of binding a cell surface receptor. The ligand can be a protein, polypeptide, glycoprotein, glycopeptide or glycolipid which has functional groups that are exposed sufficiently to be recognized by the cell surface component. It can also be a component of a biological organism such as a virus, cells (e.g., mammalian, bacterial, protozoan).

Alternatively, the ligand can comprise an antibody, antibody fragment (e.g., an F(ab′)₂ fragment) or analogues thereof (e.g., single chain antibodies) which binds the cell surface component (see e.g., Chen et al. (1994) FEBS Letters 338:167-169, Ferkol et al. (1993) J. Clin. Invest. 92:2394-2400, and Rojanasakul et al. (1994) Pharmaceutical Res. 11(12):1731-1736). Such antibodies can be produced by standard procedures.

Useful ligands will vary according to the particular cell to be targeted. For targeting hepatocytes, proteins and polypeptides containing galactose-terminal carbohydrates, such as carbohydrate trees obtained from natural glycoproteins, can be used. For example, natural glycoproteins that either contain terminal galactose residues or can be enzymatically treated to expose terminal galactose residues (e.g., by chemical or enzymatic desialylation) can be used. In one embodiment, the ligand is an asialoglycoprotein, such as asialoorosomucoid, asialofetuin or desialylated vesicular stomatitis virus.

Alternatively, suitable ligands for targeting hepatocytes can be prepared by chemically coupling galactose-terminal carbohydrates (e.g., galactose, mannose, lactose, arabinogalactan etc.) to nongalactose-bearing proteins or polypeptides (e.g., polycations) by, for example, reductive lactosamination. Methods of forming a broad variety of other synthetic glycoproteins having exposed terminal galactose residues, all of which can be used to target hepatocytes, are described, for example, by Chen et al. (1994) Human Gene Therapy 5:429-435 and Ferkol et al. (1993) FASEB 7: 1081-1091 (galactosylation of polycationic histones and albumins using EDC); Perales et al. (1994) PNAS 91:4086-4090 and Midoux et al. (1993) Nucleic Acids Research 21(4):871-878 (lactosylation and galactosylation of polylysine using α-D-galactopyranosyl phenylisothiocyanate and 4-isothiocyanatophenyl β-D-lactoside); Martinez-Fong (1994) Hepatology 20(6):1602-1608 (lactosylation of polylysine using sodium cyanoborohydride and preparation of asialofetuin-polylysine conjugates using SPDP); and Plank et al. (1992) Bioconjugate Chem. 3:533-539 (reductive coupling of four terminal galactose residues to a synthetic carrier peptide, followed by linking the carrier to polylysine using SPDP).

For targeting the polyplex to other cell surface receptors, the surface domain of the polyplex can comprise other types of ligands. For example, mannose can be used to target macrophages (lymphoma) and Kupffer cells, mannose, 6-phosphate glycoproteins can be used to target fibroblasts (fibro-sarcoma), intrinsic factor-vitamin B12 and bile acids (See Kramer et al. (1992) J. Biol. Chem. 267:18598-18604) can be used to target enterocytes, insulin can be used to target fat cells and muscle cells (see e.g., Rosenkranz et al. (1992) Experimental Cell Research 199:323-329 and Huckett et al. (1990) Chemical Pharmacology 40(2):253-263), transferrin can be used to target smooth muscle cells (see e.g., Wagner et al. (1990) PNAS 87:3410-3414 and U.S. Pat. No. 5,354,844 (Beug et al.)), Apolipoprotein E can be used to target nerve cells, and pulmonary surfactants, such as Protein A, can be used to target epithelial cells (see e.g., Ross et al. (1995) Human Gene Therapy 6:31-40).

Other examples of ligands include, but are not limited to, Br(CH₂)₁₀CO—NH-β-lactosyl amide, N¹-(bromoacetamide)-N¹³-(chenodeoxycholic acid amide)-4,7,10-trioxo-1,13-tridecanediamine; 1,1,1-tris-[(O¹⁶-β-D-galactopyranoside)-7,10,13,16-tetraoxo-5-one-4-aza-hexadecanyl]-1-[1-aza-11-amino-2-one-undecanyl]-methane, 1,1,1-tris-[(O¹⁶-β-D-galactopyranoside)-7,10,13,16-tetraoxo-5-one-4-aza-hexadecanyl]-1-[1,11-diaza-2,12-dione-13-bromotridecanyl]-methane, N¹-(iodoacetamide)-N¹³-(cholic acid amide)-4,7,10-trioxo-1,13-tridecanediamine, and BrCH₂CONH(CH₂)₃—O-β-cholesterol ether.

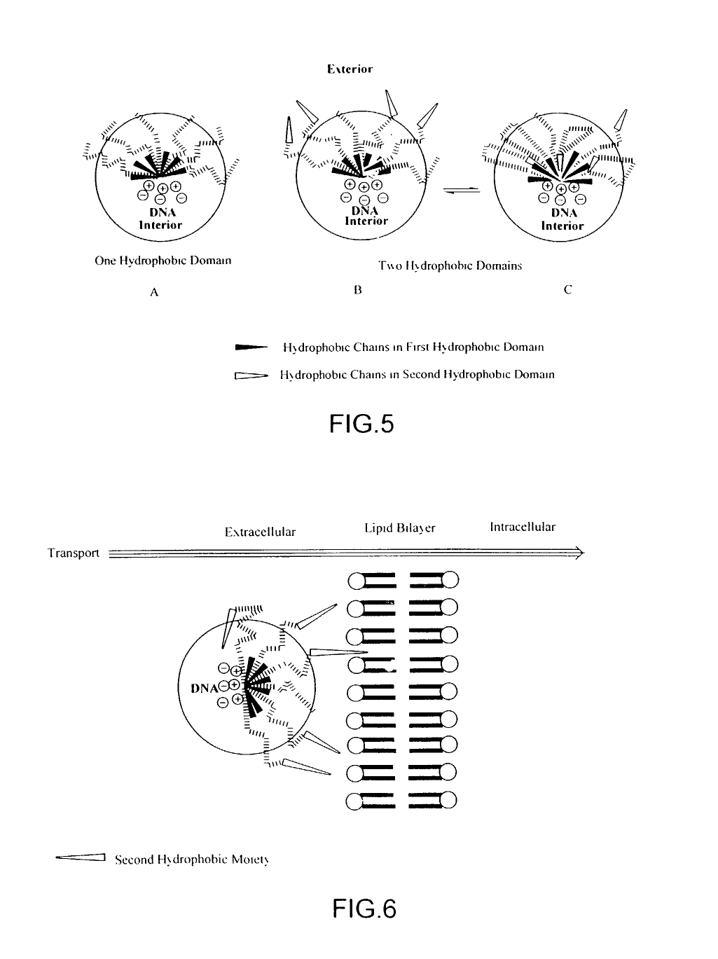

In another embodiment of the invention, polyplexes containing co-polymer domains having one or more hydrophobic moieties are able to interact, e.g., bind, with particular formulants and fuse with cellular membranes. Such polyplexes (formed with copolymers comprising one or more hydrophobic moieties) are shown in FIGS. 5a and b, respectively. In these figures, the equilibrium of the hydrophobic moieties in the second domain between a free state (FIG. 5b) and a bound state (FIG. 5c) is depicted. This mechanism of equilibration between free and bound states may permit some population of free state form to enhance a docking and fusing step that may be required for cellular entry (FIG. 6). This equilibrium can be modulated by relative strength of hydrophobic moieties within the hydrophobic domains of the polyplexes.

The “cationic moiety” or “cationic backbone moiety” of the copolymers which make up the cationic domain of the polyplex can include any moiety capable of electrostatically interacting with the anionic agent (e.g., negatively charged polynucleotides). Preferred cationic moieties for use in the carrier include non-peptidic and peptidic polycations, such as polylysine (e.g., poly-L-lysine), polyarginine, polyomithine, spermine, basic proteins such as histones (Chen et al., supra.), avidin, protamines (see e.g., Wagner et al., supra.), modified albumin (i.e., N-acylurea albumin) (see e.g., Huckett et al., supra.) and polyamidoamine cascade polymers (see e.g., Haensler et al. (1993) Bioconjugate Chem. 4: 372-379). A preferred polycation is polylysine (e.g., ranging from about 2,000 to about 80,000 daltons, from about 3,800 to about 60,000 daltons, or from about 5,000 to about 50,000 daltons). Examples of non-peptidic cationic backbone moities include peptoids (e.g., polymers comprised of modified amino acids or other peptide like polymers) and polyalkylenimines, such as polyethylenimine and polypropylenimine.

In one embodiment, the cationic backbone moiety comprises polylysine having a molecular weight of about 17,000 daltons (purchased as the hydrogen bromide salt having a MW of a 26,000 daltons), corresponding to a chain length of approximately 100-120 lysine residues. In another embodiment, the cationic backbone moiety comprises a polycation having a molecular weight of about 2,600 daltons (purchased as the hydrogen bromide salt having a MW of a 4,000 daltons), corresponding to a chain length of approximately 15-10 lysine residues.

The term “hydrophobic moiety” includes moieties which make up the hydrophobic domain of the polyplex. Hydrophobic moieties may be selected based on their fusogenic properties or their interactions with components of cellular membranes, such as lectins and lipid head groups. In one embodiment, the hydrophobic moiety comprises linear or branched polymers, linear branched or cyclic, aliphatic, alkenyl, alkynyl groups, aromatic groups or combinations thereof. The hydrophobic moiety may comprise one or more heteroatoms heterocyclic groups, peptides, peptoids, natural products, synthetic compounds, steroids, and steroid derivatives (e.g., hydrophobic moieties which comprise a steroidal nucleus, e.g., a cholesterol ring system) and/or other hydrophobic moieties known in the art which enable the polyplex to perform its function, e.g., deliver an anionic agent across a cell membrane. Delivery of polyplexes also may be further enhanced using permeation enhancers. In a further embodiment, the hydrophobic moiety contains from about 4 to 40 carbon atoms. In another embodiment, about 0.5% to about 85% of cationic charges on the cationic backbone are modified by hydrophobic moieties. The hydrophobic groups may be, for example, charged, neutral, ligand bearing, polymeric, polypeptidic, peptoidic, or polypeptoidic. Examples of hydrophobic moieties include poly-(C18-S-Cys) and poly (Phe). In a further embodiment, the hydrophobic domain may be absent.

The term “hydrophilic domain” or “hydrophilic moieties” may be selected such that the polyplex is capable of performing its intended function, e.g., deliver anionic agents through lipid membranes. Examples of hydrophilic moieties which comprise the hydrophilic domains of the polyplexes include polymers such as, for example, polyethers, such as poly(oxyalkylene glycol) (e.g., poly(oxyethylene glycol) (PEG), or poly(oxypropylene glycol), etc.). Other examples of hydrophilic moieties include polyheterocyclic polymers, such as poly(ethyloxazoline) and poly(methyloxazoline). In an embodiment, the mass ratio of the hydrophilic moieties to the cationic backbone moiety is from about 1:1 to about 40:1. Other hydrophilic moieties are described in greater detail below.

II Co-Polymers Used to Form Polyplexes

Polyplexes of the invention can be formed using a variety of co-polymers arranged and combined to form several different “architectures” suitable for cell delivery. These co-polymers include, for example, block co-polymers and random graft co-polymers, and may also include other chemical or biological constructs which are useful for cell delivery (e.g., peptides or other cellular ligands as described in the previous subsection).

A. Block Co-polymers

Polyplexes can be formed using block co-polymers of the formula (I):

A-B-C (I)

wherein A is a hydrophilic moiety, B is a hydrophobic moiety; and C is a cationic backbone moiety. In certain embodiments, the block copolymers may also comprise one or more additional hydrophobic and/or hydrophilic moieties.

In an embodiment, the polyplex of the invention is comprised of one or more copolymers of the formula (I). Generally, the cationic backbone moiety of one or more copolymers of formula (I) interact with an anionic agent, as described above, to form the cationic domain of the polyplex. The hydrophobic moieties of the copolymer(s) interact to form the transitional or hydrophobic domain and the hydrophilic moieties of the copolymers interact to form the hydrophilic (e.g., suface domain) of the polyplex.

In an embodiment, the invention uses hydrophilic PEG chains grafted onto through hydrophobic moieties to cationic backbone moieties which evade the reticuloendothelial system. The hydrophilic PEG polymer moieties also minimize serum effects and extend circulation. Furthermore hydrophobic moiety of the block co-polymer also generates a protective “hydrophobic shell” around the anionic agent (e.g., DNA) during polyplex formation.

Previously described co-polymers posed the disadvantage of having various chains grafted onto a cationic domain (e.g., poly-L-lysine) via a lysine ε-amino group. These grafted chains introduced steric hindrance to DNA binding and limit the grafted co-polymer architecture. In contrast, polyplexes of the present invention are formed with co-polymers which reduce the amount of steric hindrance of the anionic agent by using block co-polymers having unmodified cationic domains (except, in certain embodiments, at the two terminal ends) which, thus, can be added or built onto (e.g., other blocks (domains, moieties) can be added on).

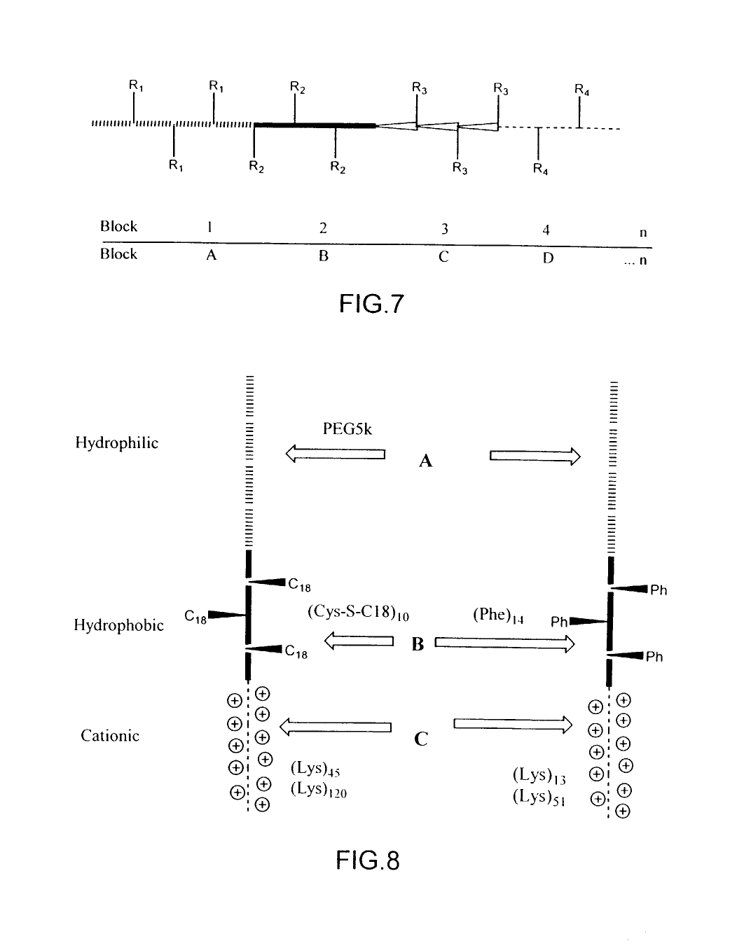

This concept, as used herein, is referred to as “block architecture” of co-polymers. Each block is synthesized by a sequential polymerization of appropriate monomers. The initiation step involves the first block (block A) that has a functional group ready to start a polymerization of a monomer B for the second block (block B). Once the supply of monomer B is exhausted and block B polymerization is completed, the second monomer C can be added and polymerization continued until completion. The entire stepwise polymerization can be repeated any number of times until desired composition of block co-polymer is achieved. Each block can then be modified by substituents to further modulate properties of polymers. As shown in FIG. 7, each individual block (domain), as designated by either a number or a letter, may have additional substituents as shown (R₁ through Rn). These substituents may or may not be equal to each other R₁≠R₂≠R₃≠ . . . ≠Rn) in each individual domain.

The use of block co-polymers to form polyplexes of the invention is shown in FIG. 8. The constituent chains of the block co-polymer can span the cationic, transitional (e.g., hydrophobic), and/or surface (e.g., hydrophilic) domains. Advantageously, the block copolymers also can be designed in such a way as to create interactions, such as hydrophobic interactions, between the domains that may promote a “closed shell” upon polyplex formation with an anionic agent, such as DNA. Other chemical interactions that may be used to close the shell upon polyplex formation are electrostatic interactions, hydrogen bonding, Van der Waals interactions, ionic interactions, and metal ion complexation. Such interactions can stabilize the interactions between the cationic domain and the anionic agent, such that the cationic moieties assemble near the anionic agent due to the cooperative nature of interactions of closing the shell, and thereby forming the polyplex.

The properties of hydrophobic interactions may be modulated in this design by varying the ratios of hydrophobic monomer to initiator during polymerization. This design allows for selection of monomers with stronger or weaker hydrophobes. Examples of synthesized block co-polymers are presented in Table 1. Monomers may be prepared by literature methods (Daly et al Tet. Lett. 1988 29(46), 5859; Kataoka, K. Macromolecules, 1995, 28, 5294; Blessing et al. PNAS, 1998, 95, 1427). Cysteine-S—C18 was prepared as described in the examples. Polymerization was conducted by adopting literature procedure. MeO-PEG5k-NH2 was used to initiate polymerization of monomer aminoacid anhydride.

TABLE 1
Examples of Block Co-polymers of the Invention

| | | | Block B | Stoichio- | ¹H | | Stoichio- | ¹H | Calcu- | Effective |
|---|---|---|---|---|---|---|---|---|---|---|
| | | Bloc | Hydro- | metric | NMR | Block C | metric | NMR | lated | Diameter |
| | | A Size | phobic | Ratio | Ratio | Cationic | Ratio | Ratio | MW | [nm] at |
| ID | BLOCK A | ×10³ | Domain | m/PEG | m/PEG | Domain | n/PEG | n/PEG | [g/mol] | +/−1.35 |
| BP-A | PEG-NH2 | 5 | (Cys—S—C₁₈)ₘ | 10 | 10 | (Lys)n | 45 | 45 | 17950 | 230.7 |
| BP-B | PEG-NH2 | 5 | (Cys—S—C₁₈)ₘ | 10 | 10 | (Lys)n | 120 | 120 | 34550 | 142.8 |
| BP-C | PEG-NH2 | 5 | (Phe)ₘ | 10 | 14 | (Lys)n | 50 | 51 | 16136 | 92.1 |
| BP-D | PEG-NH2 | 5 | (Phe)ₘ | 10 | 14 | (Lys)n | 10 | 13 | 9372 | 97.5 |
| BP-E | PEG-NH2 | 5 | None | NA | NA | (Lys)n | 20 | 3 | 9200 | Not Done |
| BP-F | PEG-NH2 | 5 | None | NA | NA | (Lys)n | 120 | 120 | 30200 | 107.4 |
| BP-G | PEG-NH2 | 5 | None | NA | NA | (Lys)n | 20 | 20 | 9200 | 102.8 |

NA—Not Applicable

Examples of block copolymers of the invention include those given in Table 1 above as well as block copolymers of the formula PEG1-20k-block-(CysC₁₈)₈₋₁₂-block-(Lys)₁₀₋₁₄₀, such as PEG5k-block-(CysC₁₈)₁₀-block-(Lys)₄₅ and PEG5k-block-(CysC₁₈)₁₀-block-(Lys)₁₂₀.

B. Random Grafts

Polyplexes of the invention can also comprise copolymers which have been formed by the random graft method. In an embodiment, the copolymers synthesized by the random graft method are of the formula:

(A)ₙ—C-(B)ₓ (II)

wherein A is a hydrophilic moiety, B is a hydrophobic moiety, C is a cationic backbone moiety, and n and x are values which can be selected such that the resulting polyplex is capable of performing its intended function (e.g., values of x and n may each range independently from 0 to 1000).

Typically, polymeric chains (such as ‘A’ and ‘B’ above) are grafted to amino groups on proteins, cationic polymers, or more specifically poly-L-lysine (e.g., ‘C’ above) using activated esters. The reaction of an activated ester produces an amide bond linked conjugate and, in effect, causes a net loss of charge on the conjugate. Random loss of positive charge can significantly weaken interactions with anionic agents, such as DNA. However, chemistry that leads to charge preservation on the cationic domain may have a minimal impact on interactions with anionic agents, although the interaction will be affected by also by steric hindrance of grafted chains.

Accordingly, for synthesis of random graft polymers for use in forming polyplexes of the present invention, synthetic chemistries are selected which preserve charges on the cationic domain and produce secondary and tertiary amines, as well as potentially quaternary ammonium salts. These amine species can bear a positive charge at physiological pH and, as a result, bind to anionic agents, e.g., polynucleotides, e.g., DNA. The impact of steric hindrance of grafted chains on polymer-DNA interactions can then be monitored by a fluorescence quenching assay.

The design of random graft polymers for use in polyplexes of the present invention is based, in part, on two principles. One is to preserve charge within the cationic domain. The second is to introduce one or more hydrophobic domains into the polyplex to stabilize the polyplex and to allow for interaction with hydrophobic formulants (e.g., penetration enhancers) which interact with these domains through hydrophobic interactions. Ligand-mediated cell targeting also can be used by hydrophobic association of the ligand with the hydrophobic domain and/or a conjugate with an engineered hydrophobic domain.

Advantageously, partially hydrophobic conjugates also may be used since they possess moieties that preserve sufficient water solubility (since purely hydrophobic molecules are water insoluble). These conjugates can be made up of two different types of grafts, hydrophilic moieties to maintain adequate water solubility (‘A’), and hydrophobic moieties (‘B’) to introduce a domain with binding and micelle formation properties. In one embodiment, the polymer is designed by grafting two or more of these elements onto a cationic backbone moiety (e.g., a cationic polymer, ‘C’). A suitable grafting element, or hydrophilic moiety for this approach is PEG, which promotes solubility and steric shielding. Another suitable grafting element is any hydrophobic moiety, as described above, which may form domains with binding capabilities. These two or more types of grafting elements can then be randomly distributed along a cationic backbone moiety during the grafting step.

As shown in Formula III below, these grafting elements can be simple or complex, and may have additional functionalities. M and K are functional groups for attachment of polymer functional domains. N is a terminal group. Y is functional group for ligand attachment or, alternatively a terminal group. The number of oxyethylene (EO) units in the hydrophilic domain is represented by c; b is represents the number of hydrophobic units in hydrophobic chain; x and n are number of hydrophilic and hydrophobic moieties attached to the cationic backbone moiety.

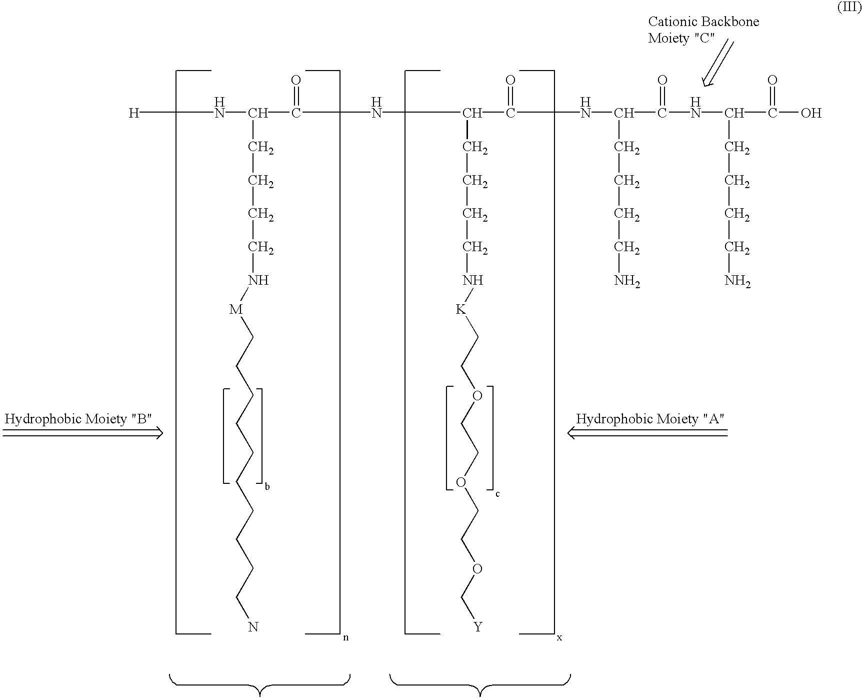

C. Polymers with Hydrophobic Moieties Grafted onto Cationic Backbone Moieties

Previously, polymer complexes have been used successfully for gene delivery in vitro. However, in vivo applications suffer from a variety of serum effects that lower the overall gene delivery efficiency.

In an effort to boost expression levels, polymers have been synthesized with multiple domains. However, as previously discussed, such polymers suffer (due to a large percent of substitution of amino groups on the cationic domain) from increased steric hindrance for DNA binding.

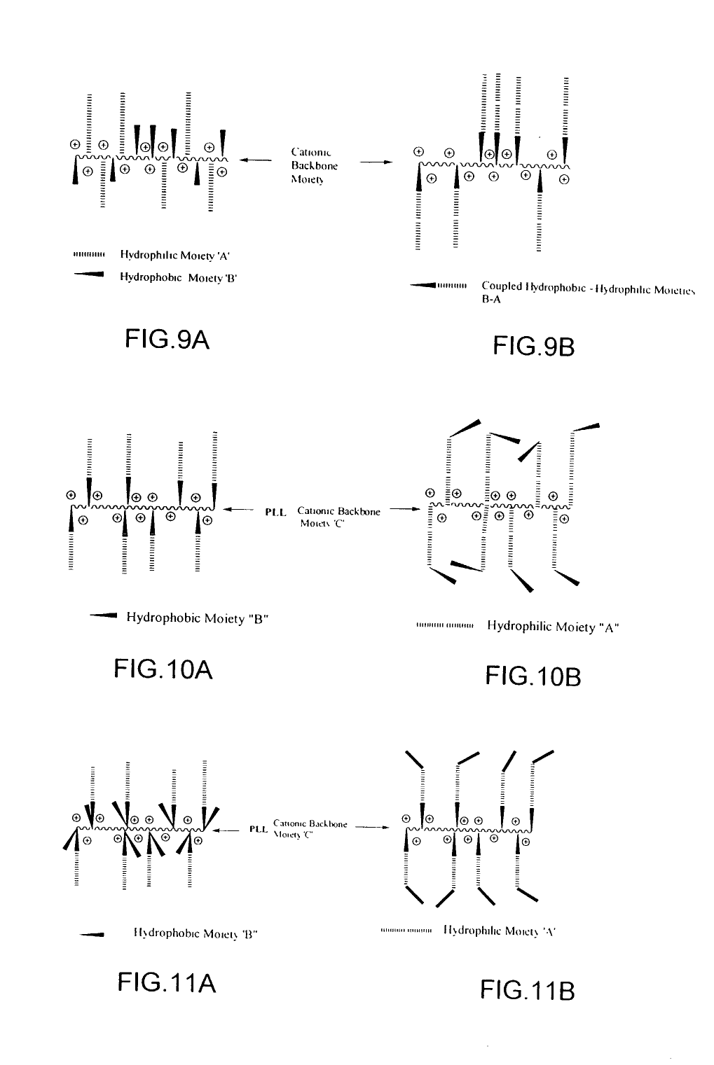

As part of the present invention, it was discovered that a high percent modification and position of a hydrophobic moiety with respect to cationic and hydrophilic moieties plays a significant role in terms of the properties of polyplexes made up of multiple co-polymer domains. These properties include, for example, solubility, size, surface properties, ligand-receptor interactions, targeting, stability characteristics and biodistribution. Thus, in an effort to lower the percent modification of cationic backbone, a new strategy was devised. In this strategy, instead of grafting independently different kinds of molecules (hydrophilic and hydrophobic moieties) on the cationic backbone moiety, these moieties are coupled into one hybrid grafting element first (hydrophobic moieties (‘B’) coupled to hydrophilic moieties (‘A’), and then the resulting hybrid element is grafted onto the cationic back bone moiety (FIG. 9a vs 9b).

One novel strategy of the present invention (i.e., coupling hydrophilic-hydrophobic grafting elements) reduces the percent modification by half and results in stronger polyplexes. It also allows one to vary the position of the hydrophobic moieties with respect to the cationic and hydrophilic moieties (FIGS. 10a and 10b).

Such hydrophobic domains can be engineered onto cationic backbone moieties using several different methods. First, the hydrophobic moieties may be positioned between the cationic backbone and the hydrophilic moieties (FIG. 10a). Alternatively, they may be attached at the terminus of the hydrophilic domain, which then may be grafted onto a cationic backbone moiety (FIG. 10b). Moreover, any of these hydrophobic moieties can be made “more hydrophobic” by increasing the number of hydrophobic moieties per individual grafting element (FIG. 11a vs b). Such hydrophobic moieties also may incorporate natural and synthetic polymers, substituted and unsubstituted linear, branched, aliphatic, alkenyl, and alkynyl groups. The hydrophobic moieties may also include heterocyclic and carbocyclic groups, as well as combinations of groups. The hydrophobic moiety can be any moiety which allows the polyplex to perform its intended function. Furthermore, the overall hydrophobicity of these conjugates can be modulated by changes in grafting densities as well as the substitution and chemical makeup of the hydrophobic moieties.

In an embodiment, the polyplexes of the invention comprise copolymers formed by the graft method. In an embodiment, the copolymers synthesized by the graft method are represented by formula IV, below:

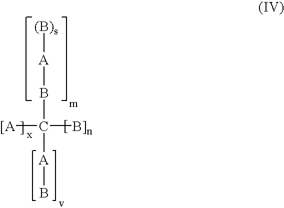

wherein each A is an independently selected hydrophobic moiety, each B is an independently selected hydrophilic moiety, C is a cationic backbone moiety and m, n, s, v, and x which are selected such that the resulting polyplex is capable of performing its intended function (e.g., values of m, n, v, s and x may each range independently from 0 to 1000).

Examples of cationic backbone moieties (‘C’) include poly-L-lysine (PLL) polyethylenimine.

Examples of hydrophobic moieties (‘B’) include alkyl groups having from about 2 to about 80 carbon atoms, alkyl groups having from about 4 to about 40 carbon atoms, etc, cholesterol derivatives, hydroxybenzyl-amidine, biphenyl, cholic acid derivative Trigal-NH(CO)CH₂Br lactose-(CO)—C₁₂—Br picolyl-Cl, or chenodeoxycholic acid-Br.

Examples of hydrophilic moieties (‘A’) include-(ε-NH-PEG2-8k)₁₀₋₂₀, (ε-NH—C10-Igepal-CO-990)₂₋₁₀, (ε-NH-Brij98)₇₋₂₀, nd (-ε-NH—CH₂CH(OH)CH₂O(PO)₃₀₋₈₀(EO)₈₀₋₁₅₀OCH₃)₅₋₃₀, TritonX-405-C₁₀—Br PEG5k-C₁₂—Br, Igepal-C₁₀—Br, PEG0.75k-C₁₀—Br C₁₈-PEG4.4k-Br C₁₈-PEG5k-C₁₀—Br N—(C₁₀-PEG2k)—N—(C₁₂)—N—(COCH₂I) PEG2k-C₁₀—Br, and PEG-Epoxide.

Examples of copolymers which may be used to form the polyplexes of the invention include, but are not limited to, poly-L-lysine-graft-(ε-NH-PEG2-8k)₁₀₋₂₀ t-(ε-NH—CH₂CONHCH₂CH₂CH₂—O-β-cholesterol ether)₁₀₋₄₀; poly-L-lysine-graft-(ε-NH-PEG2-8k)₁₀₋₂₀(ε-NH—(CH₂)₅₋₂₀—CO—NH-Lactose)₅₋₂₀; and PLL5-10k-graft-(ε-NH—C10-PEG2k)₁₋₁₀-graft-(ε-NH—CH₂CONH(CH₂)₅₋₂₀—CO—NH-Trigalactose)₅₋₂₀. Other examples include poly-L-lysine-graft-(ε-NH-PEG5k)_{12.8}-graft-(ε-NH-CH₂CONHCH₂CH₂CH₂—O-β-cholesterol ether)₂₆; poly-L-lysine9.6k-graft-(ε-NH-PEG3k)_{12.6}-graft-(ε-NH—(CH₂)₁₀-CO—NH-Lactose)_{8.8}; and PLL9.4k-graft-(ε-NH—C10-PEG2k)_{4.7}-graft-(ε-NH—CH₂CONH(CH₂)₁₀-CO—NH-Trigalactose)₉.

Other examples of polymers which the polyplexes of the invention may be comprised of include poly-L-Lysine-graft-(ε-NH—C10-PEG2k)₅₋₁₅, poly-L-Lysine-graft (ε-NH—C1-Triton X-405)₅₋₁₅, PLL-graft-(ε-NH—C10-Igepal-CO-990)₂₋₁₀; PLL-graft-(ε-NH-Brij700)₂₋₁₀; PLL-graft-(ε-NH—C10-Brij700)₄₋₁₅; PLL-graft-(ε-NH—CH₂CH(OH)(CH₂)₉-PEG)₄₋₁₅; PLL-graft-(ε-NH-Brij98)₂₋₂₀; PLL-graft-(NH-Brij98)₄₋₁₀; PLL-(-ε-NH—CH₂CH(OH)CH₂O(PO)₃₀₋₈₀(EO)₈₀₋₁₅₀OCH₃)₅₋₃₀; or polyethylenimine-graft-(—NH—CH₂CH(OH)CH₂O(PO)₃₀₋₈₀(EO)₈₀₋₁₅₀OCH₃)₅₋₃₀.

In a further embodiment, the polymers of which the polyplexes of the invention are comprised are PLL10k-graft-(ε-NH—C10-PEG2k)₉; PLL10k-graft-(ε-NH—C10-Triton X-405)₉; PLL9.4k-graft-(ε-NH—C10-Igepal-CO-990)_{3.2}; PLL9.4k-graft-(ε-NH-Brij700)_{2.8}; PLL9.4k-graft-(ε-NH—C10-Brij700)_{6.6}; PLL9.4k-graft-(ε-NH—CH₂CH(OH)(CH₂)₉-PEG5k)_{6 5}; PLL9.4k-graft-(ε-NH-Brij 98)₁₁; PLL9.4k-graft-(NH-Brij98)₆; PLL9.4k-graft-(-ε-NH—CH₂CH(OH)CH₂O(PO)₆₁(EO)₁₁₃OCH₃)_{9.8}; PLL9.4k-graft-(ε-NH—CH₂CH(OH)CH₂O(PO)₆₁(EO)₁₁₃OCH₃)_{24.6}; polyethylenimine-graft-(—NH—CH₂CH(OH)CH₂O(PO)₆₁(EO)₁₁₃OCH₃)₇; or polyethylenimine-graft-(—NH—CH₂CH(OH)CH₂O(PO)₆₁(EO)₁₁₃OCH₃)₁₅.

III Formulants, Surfactants, and Other Penetration Enhancers Which Can be Used in Conjunction with Polyplexes or Free Nucleic Acids

In another aspect, the the invention provides various penetration enhancers, such as formulants and surfactants, which can be used in combination with polyplexes of the invention, or in combination with free (i.e., uncomplexed) anionic agents (e.g., free DNA), to deliver the anionic agents across lipid membranes and cellular boundaries. Furthermore, the penetration enhancers can be used in concert with nucleic acid, alone or with a polyplex formulation, to enhance expression of the nucleic acid.

Many drugs are present in solution in both ionized and nonionized forms. However, sometimes only lipid soluble or lipophilic drugs readily cross cell membranes or other lipid membranes. It has been discovered that even non-lipophilic drugs may cross cell membranes if the membrane to be crossed is treated with a penetration enhancer. In addition to aiding the diffusion of non-lipophilic drugs across cell membranes, penetration enhancers also enhance the permeability of lipophilic drugs. The term “penetration enhancer,” “formulant” and “surfactant” are used interchangeably herein and refer to reagents which enhance delivery of anionic agents across cellular boundaries, alone or in conjunction with a polyplex of the invention. In a particular embodiment, the invention employs DHPC as a penetration enhancer.

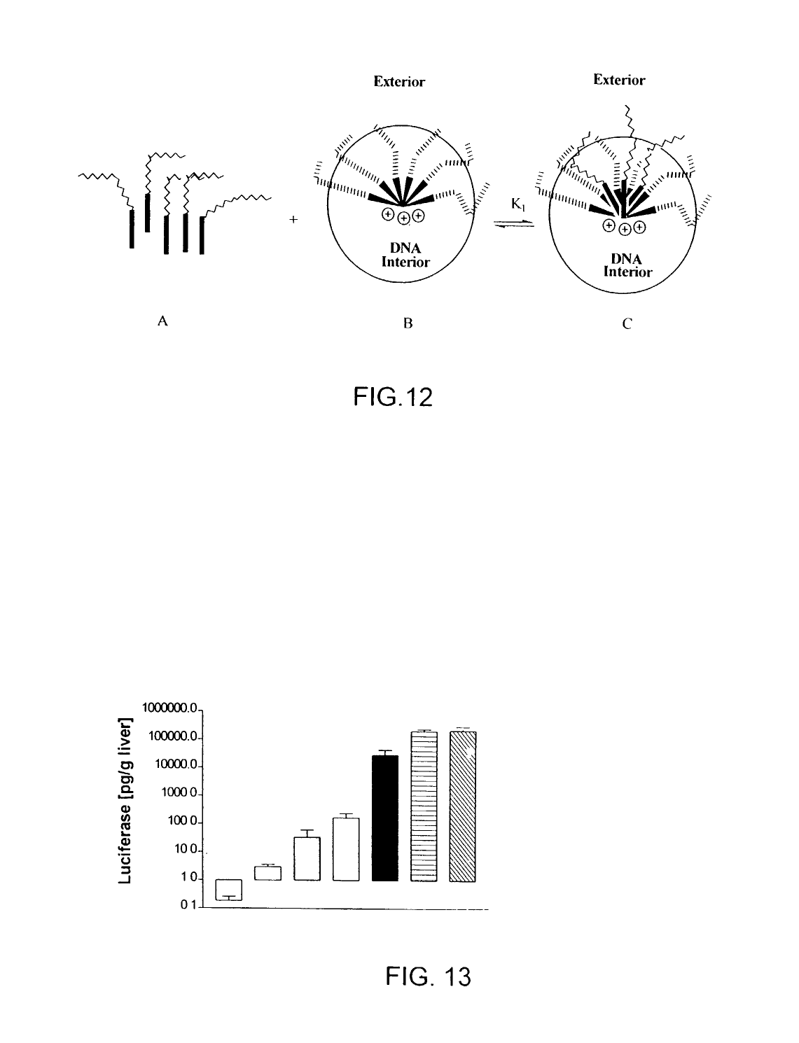

As shown in FIG. 12, during polyplex formation, penetration enhancers (e.g., formulants) may interact with polyplex. An equilibrium between the interacting formulant and the free formulant is established and is represented by the equilibrium constant, K₁. The equilibrium constant can be modulated, for example, by modifying the strength of hydrophobic domain present in the polyplex or by modifying the hydrophobic domain of the penetration enhancer molecule itself (if applicable). The equilibrium also can be shifted depending on the structure of the pentration enhancer and the architecture of the polyplex and its constituent co-polymers and their hydrophobic moieties. The formulation process may also be accomplished in a stepwise manner. For example, a penetration enhancer may be equilibrated with a copolymer followed by the addition of anionic agent. Stronger penetration enhancers may form stronger co-micelles with conjugate verses polyplex and may in effect release DNA from the polyplex. The DNA release is both conjugate and formulant dependent and can be monitored by a fluorescence DNA release assay.

Under ideal conditions, after administration by, for example, injection of the polyplex (e.g., and the penetration enhancer) and arrival of the polyplex near or at the target cell surface, the penetration enhancer will be released and will equilibrate between its polyplex bound form, free form, and cell surface (membrane) bound form, as shown in FIG. 12. Once pentration enhancer-cell surface interactions commence, the lipid bilayer membrane permeability changes, resulting in enhanced internalization, i.e., cellular uptake. In the case where the polyplex encapsulates nucleic acid (e.g., DNA), enhanced cellular uptake correlates with enhanced levels of expression.

Accordingly, in another aspect, the invention provides methods and compositions for enhancing delivery of anionic agents, e.g., polynucleotides, through cellular membranes, by combining the anionic agent, either in the form of a polyplex or in free form (e.g., free DNA), with a formulant, surfactant, or other penetration enhancer and contacting the resulting composition with the membrane.

Examples of suitable formulants or penetration enhancers for in vitro, ex vivo or in vivo administration of anionic agents (e.g., DNA) to a subject, such as an animal or human, include, for example, non-ionic, ethyleneoxide/propyleneoxide formulants; fluorinated type formulants; non-ionic carbohydrate and polyol formulants; ionic negatively charged formulants; bile acids and their derivatives and salts; ionic, cationic and zwitterionic formulants; lipid derivatives; hydrophobes; and other formulants.

Specifically, suitable non-ionic, ethyleneoxide/propyleneoxide type formulants or penetration enhancers include: Brij surfactants (e.g., Brij 30, Brij 35 (C12EO23), Brij 36, Brij 52, Brij 56, Brij 58, Brij 72, Brij 76, Brij 78, Brij 92, Brij 96, Brij 97 (C18-1-EO10), Brij 98, Brij 98/99 (C18-1-EO20), Brij 700 (C18EO100), Brij 721 (C18EO21), 18-1-EO20), Brij 97 (C18-1-EO10 etc.), Igepal® CO-990, Igepal® DM-970, Tween 20, Tween 40, Tween 60, Tween 80, Triton X-405, Triton X-100, Tetronic 908, Cholesterol PEG 900, Span 20, Span 40, Span 85, Polyoxyethylene Ether W-1, Polypropyleneglycol monobutyl ether, Tetronic 1307, oleyl surfactants (e.g., oleyl-EO₀, oleyl-EO₂, oleyl-EO5, and oleyl-EO₁₀), azones (N-ethyl-aza-cycloheptanones, N-hexyl-aza-cycloheptanones, N-octyl-aza-cycloheptanones, N-decyl-aza-cycloheptanones, N-dodecyl-aza-cycloheptanones, N-tetradecyl-aza-cycloheptanones, and N-hexadecyl-aza-cycloheptanones) and mixtures thereof.

Examples of fluorinated type formulants include Zonyl FSN 100, Zonyl FSA, and mixtures thereof.

Examples of non-ionic, carbohydrate or polyol type formulants include D-glucopyranosides (such as n-decyl-β-, n-dodecyl-β-, n-heptyl-β-, n-octyl-β-, phenyl-β-, n-hexyl-β-, methyl-6-O—N-heptylcarbonyl-α-, n-octyl-β-, n-octyl-α-, n-octyl-racemic mixture, phenyl-β-), D-1-thioglucopyranosides (such as n-decyl-β-, n-dodecyl-β-, n-heptyl-β-, n-hexyl-β-, n-octyl-β-), D-galactopyranosides (such as n-dodecyl-β-, n-octyl-β)N-decyl-β-D-maltopyranoside, N-decanoyl-N-methyl-glucamine, N-octanoyl-N-methyl-glucamine, and mixtures thereof.

Examples of ionic (negatively charged or anionic) type formulants include: N-lauryl sarcosine salt, linolic acid salt, cholesteryl hydrogen succinate, DSPE-PEG, bile acids (e.g., natural and synthetic bile acids, conjugated bile acids, mixtures, and salts), hydrotropes (e.g., 8-(5-carboxy-4-hexyl-cyclohex-2-enyl)-octanoic acid), embonic acid, hydroxy cholic acid sodium salt, linoleic acid sodium salt, N-lauryl sarcosine sodium salt, oleic acid sodium salt, sodium lauryl sulfate and mixtures thereof.

Examples of bile acids include, but are not limited to natural and synthetic bile acids, salts, and derivatives thereof. Examples of bile acids also include lithocholate, deoxycholate, glycodeoxycholate, taurodeoxycholate, chenodeoxycholate, glycochenodeoxycholate, taurochenodeoxycholate, ursodeoxycholate, glycoursodeoxycholate, tauroursodeoxycholate, cholate, glycocholate, taurocholate, ursocholate, glycoursocholate, or tauroursocholate.

Examples of ionic, cationic or zwitterionic type formulants include cetyl pyridinium chloride monohydride, cetyltrimethylammonium bromide, DOCUSATE, N,N-dimethylheptylamine-N-oxide, N,N-dimethylnonylamine-N-oxide, N,N-dimethyloctadecylamine-N-oxide, 2-heptadecylimidazole, 2-undecylimidazole, and mixtures thereof.

Examples of lipid derivatives useful as permeation enhancers include, for example, 1,2-diheptanoyl-sn-glycero-3-phosphocholine, and 1,2-dioctanoyl-sn-glycero-3-phosphocholine, and mixtures thereof.

Examples of alcohols include, but are not limited to, aliphatic alcohols such as ethanol, N-propanol, isopropanol, butyl alcohol, and acetyl alcohol. Examples of glycols include, but are not limited to, glycerine, propyleneglycol, polyethyleneglycol and other low molecular weight glycols such as glycerol and thioglycerol. Acetates include, for example, acetic acid, gluconol acetate, and sodium acetate. Hypertonic salt solutions include sodium chloride solutions and other pharmaceutically acceptable salt solutions. Heparin-antagonists include quaternary amines, such as prolamine sulfate. Cyclooxygenase inhibitors such as sodium salicylate, salicyclic acid, and non-steroidal anti-inflammatory drugs (NSAIDS) such as indomethacin, naproxin, diclofenac are also included as penetration enhancers.

Other examples of substances useful for use as permeation enhancers include: β-carotene, chloroquine diphosphate, N-decanoyl-N-methylglucamine, DSPE-PEG, menthol, nystatin, N-octanoyl-N-methylglucamide, natural and synthetic saponins. Still other suitable formulants (penetration enhancers) for use in the invention include include alcohols, glycols, heparin antagonists, cyclooxygenase inhibitors, hypertonic salt solutions, and acetates.

Such penetration enhancers, formulants and detergents can be administered in conjunction with the anionic agent to be delivered (e.g., in the form of a polyplex of the invention or in free form), before the anionic agent, or after the anionic agent.

Advantageous penetration enhancers include N¹-(cholic acid amide)-4,7,10-trioxo-1,13-tridecanediamine, N¹-(chenodeoxycholic acid amide)-4,7,10-trioxo-1,13-tridecanediamine, and N-Chenodeoxycholyl-2-aminoethyl-phosphonic acid monopotassium salt.

A. Surfactants

Surfactants (or “surface active agents,” i.e., “detergents”) are chemical agents which, when dissolved in an aqueous solution, reduce the surface tension of the solution or the interfacial tension between the aqueous solution and another liquid, with the result that absorption of anionic agents or polyplexes of the invention interacting with such surfactants through cellular membranes is enhanced. At low concentration of surfactant, cell membrane permeability is significantly increased. As a result of increased permeability, cellular uptake of, e.g., polyplexes, can be increased. The increased cellular uptake can be observed by fluorescence histology as described in the Examples herein.

Suitable surfactants for use in the invention include, for example, bile salts and fatty acids. Other suitable surfactants include sodium lauryl sulfate, polyoxyethylene-9-lauryl ether and polyoxyethylene-20-cetyl ether (see, Lee et al. Crit. Rev. Ther. Drug Carrier Systems, 1991, p. 91); and perfluorochemical emulsions, such as FC-43 (Takahashi, et al. J. Pharm. Pharmacol. 1988 40:252). Other suitable surfactants include, for example, sodium dodecyl sulfate (SDS), lysolecithin, polysorbate 80, nonylphenoxypolyoxyethylene, lysophosphatidyl choline, polyethyleneglycol 400, polysorbate 80, polyoxyethylene ethers, polyglycol ether surfactants and DMSO.

Still other suitable surfactants include ZWITTERGENT 3-14 detergent, CHAPS (3-[(3-Cholamidopropyl)dimethylammonio]1-propanesulfonate hydrate), Big CHAP, Deoxy Big CHAP, TRITON—X-100 detergent, C12E8, Octyl-B-D-Glucopyranoside, PLURONIC-F68 detergent, TWEEN 20 detergent, and TWEEN 80 detergent.

B. Fatty Acids

Suitable fatty acids and their derivatives which can be used as penetration enhancers according to the present invention include, for example, oleic acid, lauric acid, capric acid (n-decanoic acid) myristic acid, palmitic acid, stearic acid, linoleic acid, linolenic acid, dicaproate, tricaproate, monoolein (1-monooleolyl-rac-glycerol), dilaurin, caprylic acid, arachidonic acid, glycerol 1-monocaparate, 1-dodecylazacycloheptan-2-one, acylcartinines, acycl cholines, C₁₋₁₀ alkyl esters thereof (e.g., methyl, isopropyl, and t-butyl), and mono- and di-glycerides thereof (e.g., oleate, laurate, caproate, myristate, palmiate, stearate, linoleate, etc.) (Lee et al. Crit. Rev. Ther. Drug Carrier Systems, 1991, p. 9²; Muranishi, Crit. Rev. Ther. Drug Carrier Systems, 1990, 7:1; El Hariri et al. J. Pharm. Pharmacol. 1992, 44:651).

C. Bile Salts

Natural bile salts, and their synthetic derivatives, also can be used as effective penetration enhancers according to the present invention. The term “bile salts” includes any of the naturally occurring components of bile as well as any synthetic derivatives thereof. Examples of bile salts include, for example, cholic acid (or its pharmaceutically acceptable salts, e.g., sodium cholate), dehydrocholic acid, sodium dehydrocholate, deoxycholic acid, sodium deoxycholate, glucholic acid, sodium glucholate, glycholic acid, sodium glycocholate, glycodeoxycholic acid, sodium glycodeoxycholate, taurocholic acid, sodium taurocholate, taurodeoxycholic acid, sodium taurodeoxycholate, chenodeoxycholic acid, sodium chenodeoxycholate, ursodeoxycholic acid (UDCA), sodium tauro-24,25-dihydrofusidate (STDHF), sodium glycodihydrofusidate and polyoxyethylene-9-lauryl ether (POE) (Lee et al. Crit. Rev. Ther. Drug Carrier Systems, 1991, p. 92; Muranishi, crit. Rev. in Therapeutic Drug Carrier Systems, 1990, 7:1; Yamamoto et al. J. Pharm. Exp. Ther. 1992, 263:25; Yamashita et al. J. Pharm. Sci, 1990, 79:579, and WO 99/60167).

Other examples of suitable bile acids and derivatives include natural and synthetic bile acids, their organic and inorganic salts, and conjugated bile acids and their organic and inorganic salts. Still further examples include bigchap, chaps, chapso, chenodeoxycholic acid, cholic acid methyl ester, cholesteryl hydrogen succinate, cholesteryl sulfate potassium salt, dehydrocholic acid, dehydrocholic acid sodium salt, deoxycholic acid (sodium deoxycholate), deoxy-bigchap, fusidic acid, glucholic acid(sodium glucholate), glycholic acid (sodium glycholate), glycodeoxycholic acid(sodium glycodeoxycholate), lithocholic acid, sodium tauro-24, 25-dihydro-fasidate(STDHF), sodium glycodihydrofusidate, taurocholic acid sodium salt, taurodeoxycholic acid(sodium taurodeoxycholate), taurolithocholic acid sodium salt, and ursodeoxy cholic acid.

D. Chelating Agents

Chelating agents which can be used in the present invention include compounds which remove metallic ions from solution by forming complexes with the metallic ions, resulting in absorption of the anionic agent, e.g., polynucleotides or polyplexes, through cellular membranes. Chelating agents also provide the advantage of serving as DNAase inhibitors, as most characterized DNA nucleases require a divalent metal ion for catalysis and are thus inhibited by chelating agents (Jarrett, J. Chromatogr. 1993, 613, 315). Suitable chelating agents for use in the invention include, for example, disodium ethylenediaminetetraacetate (EDTA), citric acid, salicylates (e.g., sodium salicylate, 5-methoxysalicytate and homovanilate), N-acyl derivatives of collagen, laureth-9 and N-amino acyl derivatives of β-diketones (enamines).

E. Non-Chelating Non-Surfactants Penetration Enhancers

Non-chelating, non-surfactant, formulants which can be used in the present invention include compounds which have insignificant activity as chelating agents or as surfactants, but nonetheless enhance absorption of oligonucleotides through membranes (Muranishi, Crit. Rev. in Therapeutic Drug Carrier Systems 1990, 7:1). This class of formulants (penetration enhancers) includes, for example, unsaturated cyclic ureas, 1-alkyl and 1-alkenylazacyclo-alkanone derivatives and non-steroidal anti-inflammatory agents such as dichlofenac sodium, indomethacin, and phenylbutazone.

Other agents which can be used as formulants to enhance uptake of polynucleotides or polyplexes at the cellular level include cationic lipids (such as lipofectin, U.S. Pat. No. 5,705,188), cationic glycerol derivatives and polycationic molecules, such as polylysine (WO 97/30731).

F. Other Penetration Enhancers

Other suitable penetration enhancers for use in the present invention consist of compounds having the formula:

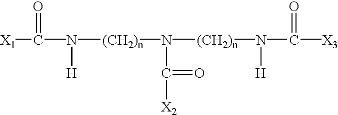

wherein n is an integer from 1 to 10, X₁ is a cholic acid group, a deoxycholic acid group, or an analog or derivative thereof, and X₂ and X₃ are each independently selected from the group consisting of a cholic acid group, a deoxycholic acid group, and a saccharide group. At least one of X₂ and X₃ is a saccharide group. Examples of saccharide groups include, for example, pentose monosaccharide groups, hexose monosaccharide groups, pentose-pentose disaccharide groups, hexose-hexose disaccharide groups, pentose-hexose disaccharide groups, and hexose-pentose disaccharide groups. For example, in a particular embodiment, the penetration enhancer has the following formula:

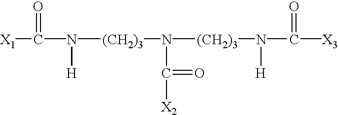

wherein X₁ and X₂ are selected from the group consisting of a cholic acid group and a deoxycholic acid group and X₃ is a saccharide group. WO 98/35554 includes other penetration enhancers of the invention.

G. Non-Chemical Penetration Enhancers

Sound waves also can be employed in conjunction with the invention to facilitate uptake of polynucleotides and polyplexes by cells.

IV Administration and Delivery of Polyplex/DNA Formulations

Polyplex and DNA formulations of the present invention can be administered to cells in vitro or in vivo (i.e., to a subject, such as a mammal) using a variety of suitable techniques known in the art, such as injection, oral administration and, in some cases, topical delivery.

In an embodiment, the invention pertains, at least in part, to a method of delivering an anionic agent through a lipid membrane. The method includes contacting the anionic agent with a delivery enhancing formulation, allowing a polyplex to form; and contacting the lipid membrane with a penetration enhancer, such that upon contact of the polyplex with the lipid membrane, the anionic agent is delivered through the membrane. The formulation comprises a cationic backbone moiety, a hydrophobic moiety, and a hydrophilic moiety;

In another embodiment, the invention also pertains to a method for enhancing expression of a nucleic acid in a cell. The method includes contacting the nucleic acid with a delivery enhancing formulation (as described above), allowing a polyplex to form, and contacting the membrane of the cell with a penetration enhancer, such that upon contact of the polyplex with the membrane of the cell, the nucleic acid is internalized into the cell and expression of said nucleic acid is enhanced. The term “enhanced” includes any expression of the nucleic acid that is greater than that observed by administering the DNA to a subject or a culture of cells with out any a polyplex or penetration assistance.

The delivery enhancing formulation is generally comprised of copolymers, as described above, with a variety of architectures which are allow the polyplex to perform its intended function, e.g., deliver an anionic agent across a lipid membrane, e.g., a cellular boundary, e.g., a cellular membrane, a nuclear membrane, an endosomal membrane, etc. For example, the delivery enhancing formulation may be a copolymer which comprises a cationic backbone moiety, a hydrophobic moiety, and a hydrophilic moiety. In an embodiment, the polyplex comprises a polymer which includes a polylysine back bone moiety, a hydrophobic moiety, and a poly(oxyethylene glycol) hydrophilic moiety.

For in vitro delivery of a polynucleotide, cultured cells can be incubated with the the compositions of the invention in an appropriate medium under conditions conducive to uptake of the compositions by the cells.

The compositions also can be delivered ex vivo to cells or tissues which have been removed from an organism, incubated the compositions of the invention, and then returned to the organism.

For in vivo delivery, compositions of the invention can be administered to a subject in a pharmaceutically acceptable vehicle. The term “pharmaceutically acceptable carrier”, as used herein, is intended to include any physiologically acceptable carrier for stabilizing the compositions invention for administration in vivo, including, for example, saline and aqueous buffer solutions, solvents, dispersion media, antibacterial and antifungal agents, isotonic and absorption delaying agents, and the like. The use of such media and agents for pharmaceutically active substances is well known in the art.

The compositions of the invention may be administered in vivo by any suitable route of administration. The appropriate dosage may vary according to the selected route of administration. The compositions are preferably injected intravenously in the form of a solution. Other suitable routes of administration include intravascular, subcutaneous (including slow-release implants), topical and oral. Appropriate dosages may be determined empirically, as is routinely practiced in the art.

The term “subject” include organisms and cells which can be adavantageously treated or altered through interaction with the polyplexes or anionic agents of the the invention. In an embodiment, the term “subject” includes protists, birds, reptiles, monera, bacteria, and preferrably, mammals, such as dogs, cats, horses, pigs, bears, cows, sheep, goats, rats, mice, hamsters, and, primates, such as chimpanzees, gorillas, and humans. In an embodiment, the subject is suffering from a genetic or an acquired disorder. Examples of disorders which the subject may be suffering from include, but are not limited to, 68. The method of claim 57, wherein the subject is treated for a disorder selected from the group consisting of hepatitis, inflammatory diseases, hemophilia, metabolic deficiencies, metabolic disorders, immune rejection of transplanted tissue, infections by invading pathogens, tissue trauma, ischemia, lipid metabolism disorders, cholesterolimia, hypercholesterolimia, peripheral and central nervous system disorders and regeneration, obesity, allergies, allergic rhinitis, asthma, Gaucher's disease, epilepsy, Parkinson's disease, ocular diseases, elevated intraocular pressure, cancer, skin disorders, and alopecia.

The term “treated,” “treating” or “treatment” includes the diminishment or alleviation of at least one symptom associated or caused by the state, disorder or disease being treated. For example, treatment can be diminishment of one or several symptoms of a disorder or complete eradication of a disorder.

The language “pharmaceutical composition” includes preparations suitable for administration to mammals, e.g., humans. When the compounds of the present invention are administered as pharmaceuticals to mammals, e.g., humans, they can be given per se or as a pharmaceutical composition containing, for example, 0.1 to 99.5% (more preferably, 0.5 to 90%) of active ingredient in combination with a pharmaceutically acceptable carrier.

The phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, α-tocopherol, and the like; and metal chelating agents, such as citric acid, ethylenediamine tetraacetic acid (EDTA), sorbitol, tartaric acid, phosphoric acid, and the like.

Formulations of the present invention include those suitable for oral, nasal, topical, transdermal, buccal, sublingual, rectal, vaginal and/or parenteral administration. The formulations may conveniently be presented in unit dosage form and may be prepared by any methods well known in the art of pharmacy. The amount of active ingredient which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol and glycerol monostearate; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluent commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert dilutents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the active compound in a polymer matrix or gel.

Pharmaceutical compositions of this invention suitable for parenteral administration comprise one or more compounds of the invention in combination with one or more pharmaceutically acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

The preparations of the present invention may be given orally, parenterally, topically, or rectally. They are of course given by forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Systemic administration is preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracistemally and topically, as by powders, ointments or drops, including buccally and sublingually.

Regardless of the route of administration selected, the anionic agent, or cationic polymeric agent of the present invention, which may be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular anionic agent or cationic polymeric agent of the present invention employed, the polyplex or penetration enhancing agent used, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses anionic agent of the invention employed in the pharmaceutical composition at levels lower than that required in order to achieve the desired therapeutic effect and gradually increase the dosage until the desired effect is achieved.

In general, a suitable daily dose of anionic agent of the invention will be that amount of the compound which is the lowest dose effective to produce a therapeutic effect. Such an effective dose will generally depend upon the factors described above. If desired, the effective daily dose of the polyplex of the invention may be administered as two, three, four, five, six or more sub-doses administered separately at appropriate intervals throughout the day, optionally, in unit dosage forms.

While it is possible for a compound of the present invention to be administered alone, it is preferable to administer the compound as a pharmaceutical composition.

The compositions of the invention may comprise compounds which may contain a basic functional group, such as amino or alkylamino, and are, thus, capable of forming pharmaceutically acceptable salts with pharmaceutically acceptable acids. The term “pharmaceutically acceptable salts” is art recognized and includes relatively non-toxic, inorganic and organic acid addition salts of compounds of the present invention. These salts can be prepared in situ during the final isolation and purification of the compounds of the invention, or by separately reacting a purified compound of the invention in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 66:1-19).

In other cases, the compounds which can be incorporated into the compositions of the present invention may contain one or more acidic functional groups and, thus, are capable of forming pharmaceutically acceptable salts with pharmaceutically acceptable bases. The term “pharmaceutically acceptable salts” in these instances includes relatively non-toxic, inorganic and organic base addition salts of compounds of the present invention. These salts can likewise be prepared in situ during the final isolation and purification of the compounds, or by separately reacting the purified compound in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like.

The term “pharmaceutically acceptable esters” refers to the relatively non-toxic, esterified products of compounds, such as, for example, penetration enhancers of the present invention. These esters can be prepared in situ during the isolation and purification, or by separately reacting the compound in its free acid form or hydroxyl with a suitable esterifying agent. Carboxylic acids can be converted into esters via treatment with an alcohol in the presence of a catalyst. Hydroxyls can be converted into esters via treatment with an esterifying agent such as alkanoyl halides. The term also includes lower hydrocarbon groups capable of being solvated under physiological conditions, e.g., alkyl esters, methyl, ethyl and propyl esters. (See, for example, Berge et al., supra.) This invention is illustrated further by the following examples which should not be construed as further limiting the subject invention. The contents of all references and published patent applications cited throughout this application are hereby incorporated by reference.

IV Polynucleotides for Cellular Delivery

The compositions (e.g., polyplexes) of the present invention can be used to deliver a variety of nucleic acids to cells, e.g., to be expressed. Polyplexes can contain more than one copy of the same polynucleotide or one or more different polynucleotides.

The term “polynucleotide”, as used herein, is intended to include any single or double-stranded DNA or RNA molecule, or any analogue thereof. In one embodiment, the polynucleotide is a gene encoding a desired therapeutic protein (e.g., a blood clotting factor, growth factor, enzyme, antagonist, immunogen, cell surface receptor or any other beneficial protein). The gene is generally in a form suitable for expression, processing and secretion by the target cell. For example, to be expressible, the gene must be operably linked to appropriate genetic regulatory elements which are functional in the target cell. Such regulatory sequences include, for example, promoter sequences which drive transcription of the gene. Suitable promoters include a broad variety of viral promoters, such as SV40 and CMV promoters. The gene may also include appropriate signal sequences which provide for trafficking of the encoded protein to intracellular destinations and/or extracellular secretion. The signal sequence may be a natural sequence of the protein or an exogenous sequence.

Regulatory sequences required for gene expression, processing and secretion are art-recognized and are selected to direct expression of the desired protein in an appropriate cell. Accordingly, the term “regulatory sequence”, as used herein, includes promoters, enhancers and other expression control elements. Such regulatory sequences are known and discussed in Goeddel, Gene expression Technology: Methods in Enzymology, p. 185, Academic Press, San Diego, Calif. (1990). The gene can be contained in an expression vector such as a plasmid or a transposable genetic element along with the genetic regulatory elements necessary for expression of the gene and secretion of the gene-encoded product.

In other embodiments of the invention, the polynucleotide is an antisense polynucleotide (DNA or RNA), or is a gene which is transcribed into an antisense RNA (e.g., a ribozyme). Antisense polynucleotides can be chemically synthesized using standard techniques well known in the art. For example, various methods of chemically synthesizing polydeoxynucleotides are known, including solid-phase synthesis which, like peptide synthesis, has been fully automated in commercially available DNA synthesizers (See e.g., Itakura et al., U.S. Pat. No. 4,598,049; Caruthers et al., U.S. Pat. No. 4,458,066; and Itakura, U.S. Pat. Nos. 4,401,796 and 4,373,071, incorporated by reference herein).

When administered in vivo, synthetic and natural polynucleotides are subject to degradation by exo- and endonucleases in a manner equivalent to any cellular nucleic acid. Accordingly, these polynucleotides can be chemically modified to provide substantial nuclease resistance. Such chemically modified polynucleotides include, for example, phosphorothioate polynucleotides, in which one of the phosphate oxygens is replaced by a sulfur atom (See e.g., U.S. Pat. No. 5,262,530, the teachings of which are incorporated by reference herein). Phosphorothioates may be synthesized using automated techniques employing either phosphoramidite or phosphonate chemistries. Other modified polynucleotides with increased stability include, for example, nonionic DNA analogs, such as alkyl- or arylphosphonates, in which the charged phosphate oxygen is replaced by an alkyl or aryl group (see e.g., U.S. Pat. No. 4,469,863, the teachings of which are incorporated by reference herein), and alkylphosphotriesters, in which the charged oxygen moiety is alkylated (See e.g., U.S. Pat. No. 5,023,243 and European Patent No: 092,574, the teachings of which are incorporated by reference herein). Both of these DNA analogs can be prepared by automated solid-phase synthesis using commercially available reagents. It is also known that addition of a diol, such as tetraethyleneglycol or hexaethyleneglycol, at either or both termini of a synthetic polynucleotide confers substantial nuclease resistance. (See e.g., U.S. Pat. No. 5,245,022, the teachings of which are incorporated by reference herein).

Examples of nucleic acids which may be incorporated into the polyplexes of the invention include nucleic acid sequences which encodes genetic markers such as, luciferase gene, β-galactosidase gene, hygromycin resistance, neomycin resistance, green fluorescent protein (GFP) or chloramphenicol acetyl transferase. Other examples of nucleic acids include sequences which encode proteins such as low density lipoprotein receptors, coagulation factors, suppressors of tumors, cytokines, angiogenesis factors, tumor antigens, immune modulators, major histocompatibility proteins, antioncogenes, p16, p53, thymidine kinase, IL2, IL4, IL10, or TNFα. Still other examples include nucleic acids which encode for viral proteins, bacterial proteins, cell surface markers, HIV antigens, HIV p24 antigens, HSVgD antigens, HBVS antigens. The nucleic acid incorporated into the polyplex of the invention also can be RNA, for example, a sense RNA, an antisense RNA, or a ribozyme.

### EXAMPLES

Materials and Methods

Poly-L-lysine (PLL) 10K [DP (Vis) 48, MW (Vis) 10,000; DP (LALLS) 32, MW (LALLS) 6,700, Mw/Mn (SEC-LALLS) 1.20], 26K [DP (Vis) 123, MW (Vis) 25,700; DP (LALLS) 120, Mw (LALLS) 25,000, Mw/Mn (SEC-LALLS) 1.20], 38K [DP(Vis) 184, Mw (Vis) 38,500; DP (LALLS) 172, Mw (LALLS) 35,900; Mw/Mn (SEC-LALLS) 1.10], Poly-L-aspartic acid (P(Asp)) sodium salt 10K [DP (Vis) 76, Mw 10,400 (Vis); DP (LALLS) 57, Mw (LALLS) 7,800] and ethidium bromide were purchased from Sigma Chemical Co., St. Louis, Mo. Tris (2-carboxyethyl) phosphine hydrochloride (TCEP.HCL) was purchased from Pierce Chemical Co. (Rockford, Ill.). PD 10 Sepadex G-25M (pre-packed) and phenyl sepharose high performance (hydrophobic interaction column [HIC]) columns and G-25M resin were purchased from Pharmacia Biotech Inc. (Piscataway, N.J.). The CM/M Poros column (CM) was purchased from PerSeptive Biosystems, Inc. (Farmington, Mass.). Synthetic polylysine, (Lys)₄₈Cys, was purchased from Dr. Christian Schwabe (Protein Chemistry Facility at the Medical University of South Carolina). Polyethylene glycol (PEG) amino 5k (MW5254; Substitution: 98% (¹H NMR), 98.2% (titration)) and Polyethylene glycol (PEG) epoxides 2K (Mₙ 1554; M_{w}/Mₙ 1.044 (GPC)), 3K (Mₙ 2696; M_{w}/Mₙ 1.035 (GPC)), and 5K (Mₙ 5231; M_{w}/Mₙ 1.017 (GPC)) were purchased from Shearwater Polymers, Inc. (Huntsville, Ala.).

The PEG amino 5k was dried in vacuo at 40° C. Acrylonitrile was purchased from Avacado Research Chemicals, Ltd., Lancaster, England. L-cysteine, 1-bromooctadecane, and LiOH.H₂O were purchased from Aldrich Chemical Co. (Milwaukee, Wis.). Potassium Hydroxide and tetrahydrofuran (THF) were purchased from VWR Scientific Products, West Chester, Pa. and double distilled from sodium benzophenyl ketal. Bis(trichloromethyl)carbonate (triphosgene) and N-ε-Z-L-lysine were purchased from Fluka Chemical Corp., Milwaukee, Wis. Plasmid DNA (pCMVb, Clontech, Palo Alto, Calif. and pCMV-Luciferase was prepared by BIO 101 (San Diego, Calif.). Plasmid DNA preparation contained more than 90% covalently closed circular DNA as determined by agarose gel electrophoresis.

Formulants, surfactants, permeation enhancers, and polymers commercially available were used as received and are listed below. Brij 700 (C18EO100), Brij 721 (C18EO21), Brij35 (C12EO23), Brij 98/99 (C18-1-EO20), Brij 97 (C18-1-EO10), Polyoxyethylene Ether W-1, Triton X-405, Triton X-100, N-Lauryl Sarcosine, sodium salt, n-Dodecyl-β-D-glucopyranoside, n-Heptyl-β-D-1-thioglucopyranoside, n-octyl-β-D-galactopyranoside, n-Decyl-β-D-maltopyranoside, Taurocholic Acid sodium salt, Saponin (from Quilaja Bark), Nystatin, and Chloroquine, diphosphate were purchased from Sigma Chemical Co. (St. Louis, Mo.). N-octyl-N-methyl glucamide, n-Octyl-α-D-glucopyranoside, n-Octyl-D-glucopyranoside, n-Octyl-β-D-1-thioglucopyranoside, N-Decanoyl-N-methyl glucamine, N,N-Dimethylnonylamine-N-oxide, N,N-Dimethyloctadecylamine-N-oxide, β-Carotene, and Cholesterol PEG-900 were purchased from Fluka Chemical Co. (Milwaukee, Wis.). Igepal CO-990, 2-Undecylimidazole, and Ethylenediamine tetrakis(propoxylate-block-ethoxylate tetrol) were purchased from Aldrich Chemical Co. Inc. (Milwaukee, Wis.). 1,2-Diheptanoyl-sn-glycero-3-phophocholine and 1,2-Dioctanoyl-sn-glycero-3-phosphocholine were purchased from Avanti Polar-Lipids, Inc. (Alabaster, Ala.). Zonyl FSN 100 and Zonyl FSA were purchased from Dupont Corp. (Wilmington, Del.). Phenyl-β-D-glucopyranosidesides and n-Octyl-β-D-glucopyranosides were purchased from Pfanstiehl Laboratories, Inc., Waukegan, Ill. N-Heptyl-β-D-glucopyranosides were purchased from Calbiochem (La Jolla, Calif.). N-Hexyl-β-D-glucopyranosides, n-Hexyl-β-D-1-thioglucopyranosides were purchased from Toronto Research Inc. (Ontario, Canada). Linolic Acid, sodium salt and 2-Heptadecylimidazole were purchased from TCI America, Inc. (Portland, Oreg.). Cholesteryl Hydrogen Succinate was purchased from ICN Biomedicals, Inc. (Aurora, Ohio). DSPE-PEG was purchased from Shearwater Polymers Inc. (Huntsville, Ala.). Tetronic 908 was purchased from BASF (Mount Olive, N.J.).

Commercially available bile acids were purchased from the following suppliers: Lithocholic acid [434-13-9], Chenodeoxycholic Acid, Glycochenodeoxycholic acid, sodium salt [16564-43-5], Deoxycholic acid [88-44-3], and Taurochenodeoxycholic acid, sodium salt [6009-98-9] were purchased from Sigma Chemical Co (St. Louis, Mo.). Glycodeoxycholic acid, sodium salt, Ursodeoxycholic acid [128-13-2], and Ursocholate were purchased from Fluka Chemical Corp. (Milwaukee, Wis.). Taurocholic acid, sodium salt [145-42-6], CHAPS [75621-03-3], and CHAPSO [82473-24-3] were purchased from Aldrich Chemical Co., Inc. (Milwaukee, Wis.). Cholic Acid [81-25-4] was purchased from Avacado Research Chemicals, Ltd. (Lancanster, England).

Instrumentation

¹H NMR spectra of the monomers and polymers were obtained on a 300 MHz ARX-300 Bruker spectrometer. IR Spectra were recorded on a Perkin-Elmer 1600 series FTIR as a KBr pellet or on NaCl plates.

Polynucleotide Carrier Complex Preparation

Polynucleotide carrier complexes were prepared by rapidly adding an equal volume of plasmid DNA to a volume of the copolymer. DNA (2×) was prepared in water and copolymers were dissolved in the 2× diluent before mixing. Polynucleotide carrier complex concentrations are reported by DNA content and were 10 μg/ml unless otherwise noted.

Exchange Reaction

Polynucleotide carrier complexes were formulated at room temperature by rapidly mixing 500 μL of DNA (2×) and 500 μL of copolymer stock solution. Final DNA concentration was 50 μg/mL at a charge ratio of 1.0 (+/−) in 150 mM NaCl. Each polynucleotide carrier complex solution was divided into five 200 μL aliquots and incubated at room temperature for 30 minutes. Anionic molecules were added to the polyplex aliquots in increasing amounts (charge ratio 1, 4, 7, 10, and 100 per phosphate group). The samples were then incubated for 20 hours and analyzed on agarose gel (0.6%).

Fluorescence Quenching Assay

The relative binding efficiencies of polycationic polymers were examined using an ethidium bromide-based quenching assay. Solutions were prepared containing 2.5 ug/ml EtBr and 10 ug/ml DNA (1:5 EtBr:DNA phosphates molar ratio) in a total volume of 1 ml. The polycation was added incrementally with fluorescence readings taken at each using a Sequoia-Turner 450 fluorometer with excitation and emission wavelengths at 540 nm and 585 nm, respectively. Fluorescence readings were adjusted to compensate for the change in volume due to addition of polycation which never exceeded 3% of the original volume. Results are reported as the percentage of fluorescence relative to that of uncomplexed plasmid DNA (no polycation).

Estimation of Polynucleotide Carrier Complex Size

Light scattering measurements were determined on a Brookhaven Instruments Corporation 90 Plus particle size analyzer equipped with a 50 mW laser which emits light at a wavelength of 532 nm. Reagents were passed through a Nalgene 200 nm surfactant-free cellulose acetate filter prior to polynucleotide carrier complex formation. Results are reported as effective diameter defined as the average diameter which is weighted by the intensity of light scattered by each carrier complex.

Gel Electrophoresis

Tris-borate EDTA urea gels were obtained through Novex (San Diego, Calif.). The gels were run in 1×TBE buffer. The samples were mixed with an equal volume of sample buffer containing 40% sucrose, 0.1% methyl green dye (Sigma Chemical Co., St. Louis, Mo.) 7.2 M urea in 1×TBE. The gels were run at 180 volts with polarity reversed for approximately 2 hours, stained with coomassie brilliant blue, and photographed.

Electron Microscopy

Carbon-coated copper grids with formvar support film (Tel Pella, Inc., Redding, CA) were glow-discharged for 30 seconds just prior to sample preparation. Samples were negatively stained with uranyl acetate by one of the following methods: 1) the grid was floated on a 15 μL droplet containing polyplexes (at 10 μg/ml unless otherwise stated) for three minutes, then wicked to filter paper. The grid was washed 2×by placing on a distilled water droplet for 15 seconds followed by thoroughly removing liquid by wicking to filter. 2) The grid was floated on a droplet containing equal volumes of sample and 1.5% uranyl acid stain (1 minute) followed by two washes with water. The grids were examined under a Zeiss EM 10b microscope at 10,000× and 40,000× magnification.

### Example 1

### Random Grafts on Cationic Chains—Synthesis of Grafting Elements

Synthesis, purification, and characterization of grafting elements can be performed as follows. In particular, the synthesis of grafting elements with a cholesterol nucleus is shown in Scheme 1. The synthesis was accomplished in three steps starting with cyanoethylation of cholesterol, followed by catalytic reduction of cyanoethyl derivative, and finally bromoacetylation of 3-aminopropyl-p-cholesterol ether.

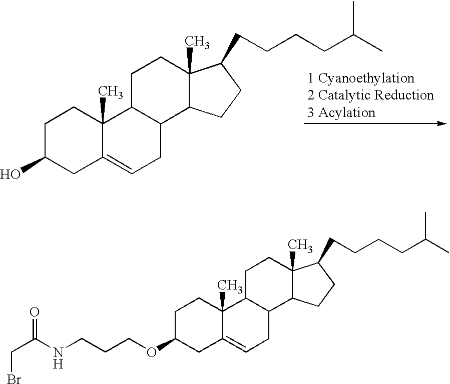

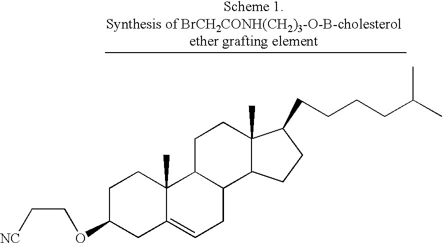

2-Cyanoethyl-O-β-cholesterol Ether

Acrylonitrile (47.7 g, 900 mmol) was added dropwise to a mixture of potassium hydroxide (0.360 g, 7.0 mmol) dissolved in water (5 mL) and cholesterol (11.6 g, 30 mmol) dissolved in 100 mL of dioxane. This mixture was stirred at room temperature for 3 days. The product was filtered, rinsed with water, methanol, and finally, cold dioxane. The product was dried in vacuo giving 5.5 g (40.3%). TLC (silica, R_{f}=0.44, CHCl₃). ¹HNMR (CDCl₃) δ 5.38 (d, 1H, CH═), 3.71 (t, 2H, OCH₂), 3.25 (h, 1H, CH₂OCH), 2.59 (t, 2H, CH₂CN), 2.40-0.86 (m, 40H), 0.69 (s, 6H, CH₃), ESP-MS of β-elimination product (C₃₀H₅₀ON—C₃H₄N) M+1: calculated, 387.7, obtained: 387.3.

3-Aminopropyl-O-β-cholesterol Ether

2-Cyanoethyl-O-β-cholesterol ether (2.02 g, 4.43 mmol) and slurry of Raney-Nickel catalyst (0.65 g) were added to a 250 mL glass hydrogenation vessel containing a solution of NaOH (20 mg) in 80 mL of 95% ethanol. The mixture was placed in a Parr Hydrogenation apparatus under hydrogen (50 psi) and shaken overnight with gentle heating. The catalyst was removed by vacuum filtration and rinsed with 95% ethanol. The solvent was evaporated in vacuo, and the residue dissolved in water. The aqueous layer was extracted with CH₂Cl₂. The organic layer was dried over Na₂SO₄, filtered through anhydrous Al₂O₃, and evaporated in vacuo. The crude product was purified by flash chromatography (neutral aluminum oxide, CHCl₃/MeOH/H₂O, 90:18:1, v/v/v) to give 1.14 g (57.8%) of a white solid. TLC (silica, R_{f}=0.12, CHCl₃/MeOH/H₂O, 90:36:1, v/v/v). ¹H NMR (CDCl₃): δ 5.34 (d, H, CH═), 3.55 (t, 2H, OCH₂), 3.13 (m, H, CH₂OCH), 2.80 (t, 2H, CH₂NH₂), 2.40-0.86 (m, 40H), 0.68 (s, 6H, CH₃), ESP-MS of M+1 (C₃₀H₅₄NO): calculated, 443.8, obtained: 443.8.

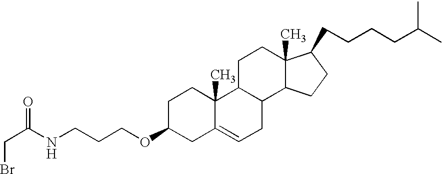

BrCH₂CONH(CH₂)₃—O-β-cholesterol Ether

3-Aminopropyl-O-β-Cholesterol ether (0.455 g, 1.03 mmol) and BrCH₂COONHS (0.294 g, 1.24 mmol) were dissolved in 10 mL of CH₂Cl₂ and the reaction mixture stirred for 1 hour. The solvent was removed in vacuo and the residue purified by flash chromatography (silica gel, CHCl₃/MeOH, 10:1, v/v) to give a white solid 0.423 g (72.7%). TLC (silica, R_{f}=0.81, CHCl₃:MeOH, 10:1, v/v). ¹H NMR (CDCl₃): δ 7.40 (b, H, NH), 5.35 (d, H, CH═), 3.90 (s, 2H, CH₂Br), 3.62 (t, 2H, OCH₂), 3.43 (m, 2H, CH₂NH₂), 3.21 (m, H, CH₂OCH), 2.40-0.86 (m, 40H), 0.68 (s, 6H, CH₃).

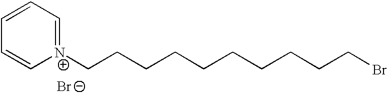

Br(CH₂)₁₀-N-pyridium Bromide.

1,10-Dibromodecane (6.0 g, 20 mmol) was dissolved in acetonitrile (10 mL) followed by addition of pyridine (0.36 g, 4.55 mmol). The resulting solution was refluxed for 5 hours. After evaporation of the solvent in vacuo, the residue was purified by flash chromatography (silica gel, EtOAc/HAc/MeOH/H₂O, 12:3:4:4, v/v/v/v) to give 1.64 g (95.2%) of a pink solid. ¹HNMR (CDCl₃): δ 9.48 [d, 2H, (CH₂)N], 8.52 (t, 1H, CH), 8.15 [m, 2H, CH₂CH₂N], 4.97 (m, 2H, CH₂N), 3.38 (t, 2H, CH₂Br), 2.06 (b, 2H, CH₂CH₂N), 1.82 (m, 2H, CH₂CH₂Br), 1.35-1.25 (b, 12H, 6×CH₂). TLC (silica, R_{f}=0.56, EtOAc/HAc/MeOH/H₂O, 12:3:4:4 v/v/v/v).

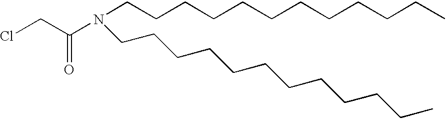

Didodecylamine Chloroacetamide

Solution of didodecylamine in CH₂Cl₂ (1.768 g, 5 mmol, 50 mL) and aqueous solution of K₂CO₃ (6.911 g, 50 mmol, 50 mL) were combined in a flask and stirred vigorously. Chloroacetic anhydride dissolved in CH₂Cl₂ (8.549 g, 50 mmol, 50 mL) was added dropwise and stirred for 6 hours. The organic layer was separated, rinsed three times with water (3×100 mL), dried overnight with MgSO₄, filtered, and evaporated in vacuo. The desired product was obtained 2.02 g (94%). TLC (silica, R_{f}=0.86, ethyl acetate). ¹H NMR (CDCl₃): δ 4.03 (s, 2H, ClCH2CO), 3.30 (m, 4H, CON(CH₂CH₂˜)₂), 1.55 (m, CON(CH₂CH₂˜)₂), 1.30 (m, 36H, —(CH₂)—), 0.90 (m, 6H, CH₃).

The synthesis of grafting element containing ligand, a terminal galactose, is shown on Scheme 2.

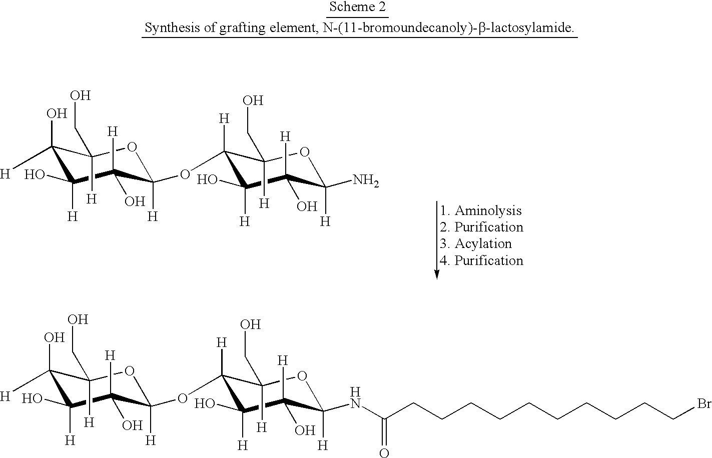

The N-(11-bromoundecanoyl)-β-lactosylamide was synthesized from α-Lactose in two steps. The first step involved formation of β-lactosyl amine that was converted to 11-bromoundecanoyl amide in the second step. The final product was used to prepare co-polymers containing terminal galactose as a potential ligand.

Br(CH₂)₁₀COONHS

DCC (2.06 g, 10 mmol) was added to a solution of 11-bromoundecanoic acid (2.65 g, 10 mmol) and N-hydroxysuccinimide (1.15 g, 10 mmol) in 40 ml of THF. The reaction mixture was stirred overnight at room temperature. The white precipitate, DCU, was removed by filtration. The solvent was removed in vacuo giving 3.38 g (9.0 mmol, 90%) of a desired product as a white solid. TLC (silica, R_{f}=0.3, CHCl₃).

¹H NMR(CDCl₃): δ 3.40 (t, 2H, BrCH₂), 2.88 (b, 4H, Suc (COCH₂CH₂CO)), 2.60 (t, 2H, CH₂COO), 1.90-1.20 (m, 16H, (CH₂)₈).

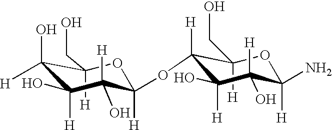

Lactosylamine

α-Lactose monohydrate (3.60 g, 10 mmol) was dissolved in 25 mL of concentrated aqueous ammonium hydroxide (16 M) to form a 0.4 M of α-Lactose solution. To a resulting solution of α-Lactose, ammonium bicarbonate was added to form a 0.4 M solution. The resulting solution was heated at 33° C. for 3 days. Then the solvent was removed in vacuo. The crude product was repeatedly dissolved in water and water was evaporated in vacuo. The entire process was repeated eight times (8×50 mL) to remove remaining ammonium salts. Finally, the resulting white solid was lyophilized to give 3.7 g of a product with 69% purity. ¹H NMR (D₂O): δ 4.39 (d, H, α-H), 4.12 (d, H, β-H). The other ¹H NMR signals were consistent with the structure of Lactose. ¹H NMR analysis also showed 20% of α-Lactose and 10% of α-Lactosylcarbamate as calculated from integration of anomeric protons. TLC (silica, R_{f}=0.3, detection with 5% H₂SO₄, EtOAc/MeOH/H₂O, 3:3:1 v/v/v).

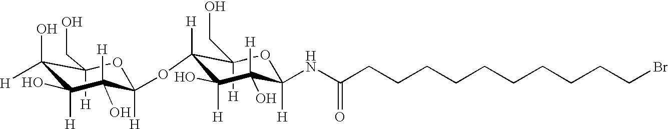

Br(CH₂)₁₀CO—NH-β-Lactosyl.

Lactosylamine (0.99 g, 2.9 mmol) and Br(CH₂)₁₀COONHS (1.05 g, 2.9 mmol) were added to DMF (20 mL). The resulting suspension was stirred at room temperature for 3 days. Then the solvent was removed in vacuo and the yellow solid was recrystalized from MeOH/H₂O (10:1, v/v) to give 0.44 g (26%) of a white product. ¹H NMR (CD₃OD): δ 4.95 (d, 1H, β-H), 4.44 (d, 1H, α-H), 4.0-3.30 (m, 10H, protons on α-Lactose without anomeric protons), 3.30 (t, 2H, BrCH₂), 2.25 (t, 2H, COCH₂), 1.90-1.35 (m, 16H, 8CH₂). TLC (silica, R_{f}=0.75, EtOAc/H₂O/MeOH 12:7:7 v/v/v).

To increase targeting via a multidentate ligand interaction with trigalactose receptor, triantennary galactose ligand was synthesized and its structure is shown below. This ligand-amine was further converted to bromoacetyl derivative that was used for synthesis of grafted co-polymers.

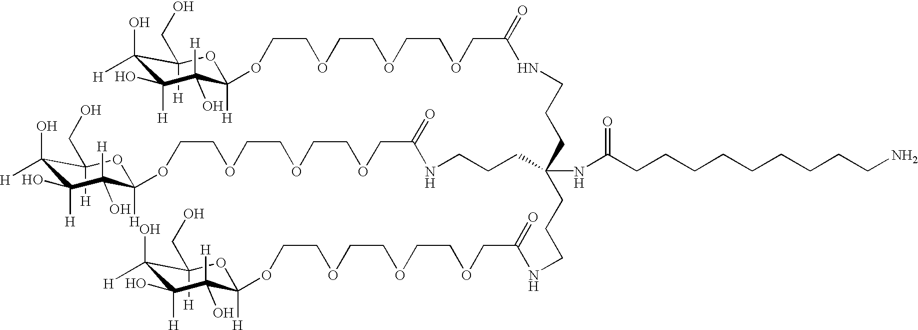

Trigalactose-ligand-amine, 1,1,1-Tris-[(O¹⁶-β-D-Galactopyranoside)-7,10,13,16-tetraoxo-5-one-4-aza-hexadecanyl]-1-[1-aza-11-amino-2-one-undecanyl]-methane (1351:070) was synthesized by Carbohydrate Synthesis Ltd. The trigalactose-ligand amine was characterized thoroughly by ¹H NMR, MS and TLC methods. The product was homogeneous by TLC on silica gel plates (R_{f}=0.12, EtOAc/HAc/MeOH/H₂O, 12:3:4:4, v/v/v/v) with approximate purity of 95%. FAB-MS, calculated for M+1, 1443.7; found for CHNO, M+1, 1443. ESP-MS, M+1, 1443.2.

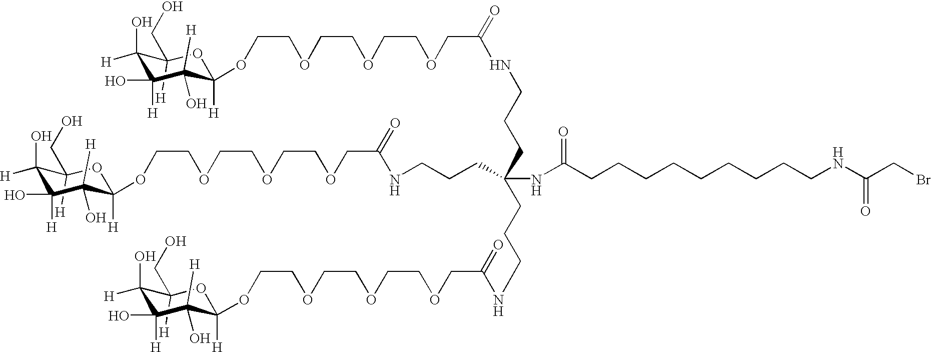

Trigalactose-ligand-bromoacetamide, 1,1,1-Tris-[(O¹⁶-β-D-Galactopyranoside)-7,10,13,16-tetraoxo-5-one-4-aza-hexadecanyl]-1-[1,11-diaza-2,12-dione-13-bromotridecanyl]-methane (1351-71), was prepared by standard procedure and used without further purification. First, trigalactose-ligand-amine (0.36 g, 0.25 mmol) was lyophilized from H₂O, then dissolved in 20 mL of MeOH, and combined with BrCH₂COONHS (0.624 g, 2.63 mmol). Triethylamine (37.6 μl, 0.27 mmol) was added and the solution was stirred overnight. The solvent was removed in vacuo and the residue purified by Sephadex G-25 column with 0.05 M Acetic Acid in 30% MeOH/H₂O v/v to give white solid (0.26 g, 0.166 mmol, yield 66.4%). The final product was lyophilized from H₂O and used for the next step without further purification. Presence of iodoacetamido moiety was confirmed by ¹H NMR (D₂O): δ 1.95 (s, 2H, COCH₂Br). The other ¹H NMR signals were consistent with trigalactose-ligand amine precursor. TLC (silica, R_{f}=0.26, EtOAc/HAc/MeOH/H₂O, 12:3:4:4 v/v/v/v).

Bile acids are transported into hepatocytes via system of protein receptors/transporters that are distinct from the ASGPr. Bile acids enter hepatocytes via a non-endocytic pathway, and therefore, can serve as a possible ligand for targeted delivery to liver. Derivatives of bile acids that can be grafted on cationic polymers were then prepared and are described below in Scheme 3.

The grafting element containing cholic acid was prepared in two steps starting with methyl cholate as shown in Scheme 3. The final product, iodoacetamide derivative was used for grafting without further purification. The polymeric products obtained in such procedure were purified by standard procedures and are described in later sections.

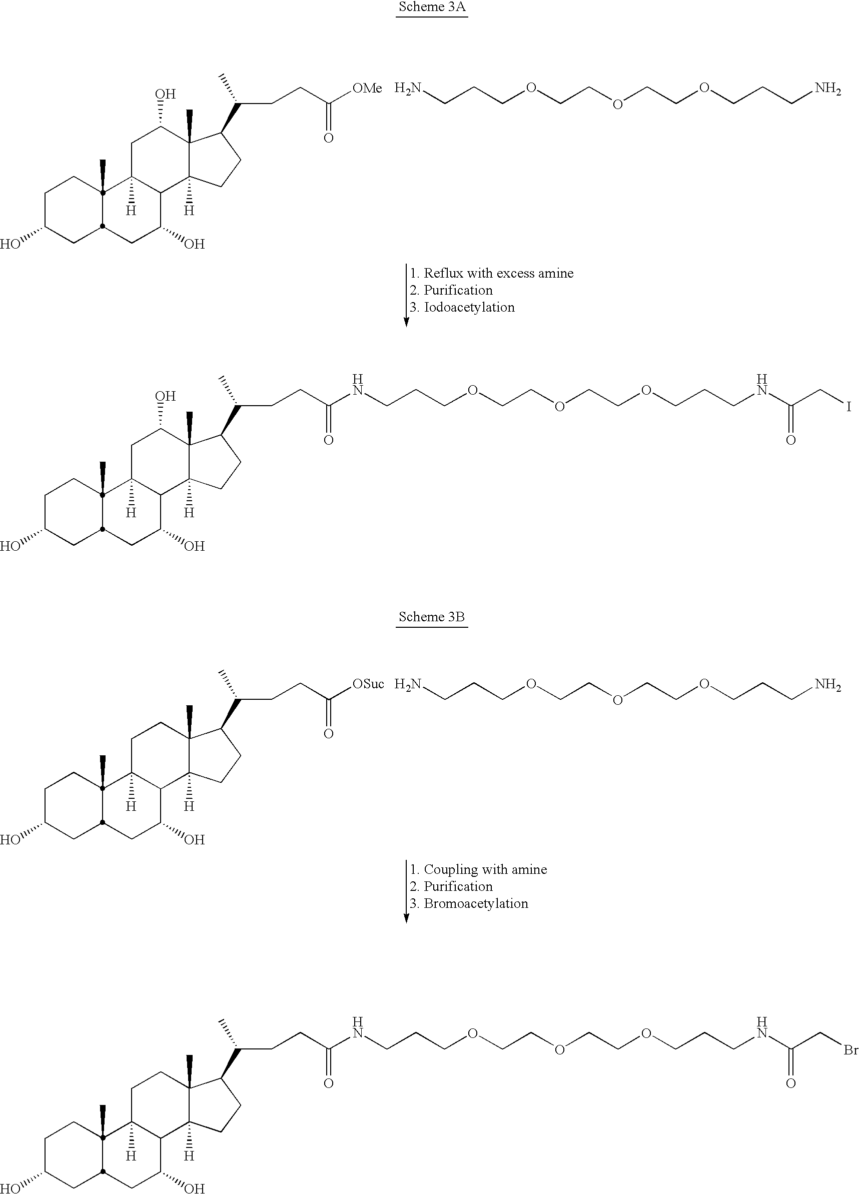

Scheme 3 depicts the synthesis of grafting elements containing bile acid derivatives. Scheme 3A depicts the synthesis of N¹-(Iodoacetamide)-N¹³-(Cholic Acid Amide)-4,7,10-trioxo-1,13-tridecanediamine) Scheme 3B depicts the synthesis of N¹-(Bromoacetamide)-N¹³-(Chenodeoxy Cholic Acid Amide)-4,7,10-trioxo-1,13-tridecanediamine.

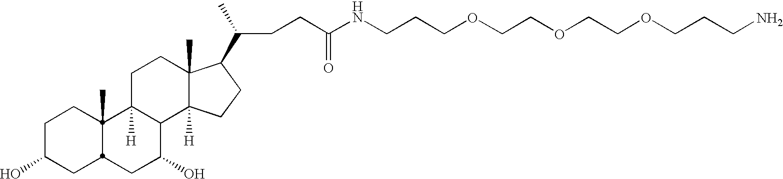

N¹-(Chenodeoxycholic Acid Amide)-4,7,10-trioxo-1,13-tridecanediamine

Chenodeoxycholic acid (5.0 g, 0.013 mole) was dissolved in dry THF (150 ml). N-hydroxysuccinimide (NHS) (1.6 g, 0.014 mole) and then Dicyclohexylcarbodiimide (DCC) (2.8 g, 0.014 mol) were added. The mixture was stirred under argon at 25° C. for 18 h. The reaction mixture was then filtered over a medium porosity sintered glass filter to remove the precipitate dicyclohexylurea (DCU). The solvents were removed in vacuo and re-dissolved in dichloromethane (200 ml). Neat 4,7,10-trioxa-1,13-tridecanediamine (70.5 g, 0.32 mol) was added and the solution stirred at 25° C. for 4 h. The reaction mixture was then washed three times with water, separated, and dried over sodium sulfate. The mixture was filtered and the solvent removed in vacuo to give a white solid (5.8 g, 75%). TLC (silica, R_{f}=0.78, i-Propanol:30% ammonium hydroxide:water, 10:2:1 v/v/v). ¹H NMR was consistent with the structure. MS for (C₃₄H₆₂N₂O₆) calcd M+1 595.8, found 595.7.

Iodoacetamide was prepared as described earlier for bromoacetamide derivative.

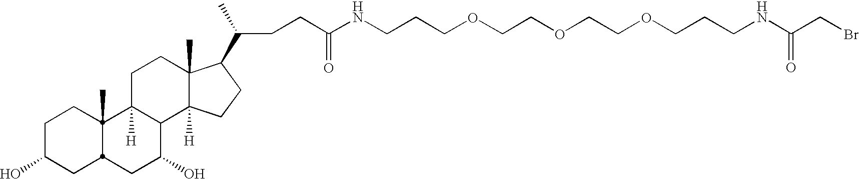

N¹-(Bromoacetamide)-N¹³-(Chenodeoxycholic Acid Amide)-4,7,10-trioxo-1,13-tridecanediamine

Chenodeoxycholic-amino derivative (1408-64) (500 mg, 0.80 mmol) was dissolved in dry methylene chloride (50 ml). Succinimidyl Bromoacetate (232 mg, 0.98 mmol) was added, and the solution stirred for 10 min. at 25° C. under argon. Methanol (10 ml) was added and the solution was stirred overnight. The solution was evaporated to dryness, re-dissolved in chloroform, and rinsed with saturated sodium chloride three times (3×200 ml). The chloroform layer was separated, dried over anhydrous sodium sulfate, and evaporated in vacuo. The product (500 mg, yield 83%) was used for the next step without further purification. TLC (silica, R_{f}=0.17, i-propanol:30% ammonium hydroxide:water, 10:2:1 v/v/v). MS for (C₃₆H₆₃N₂O₇Br) calcd M+1 715.8, found 717.7.

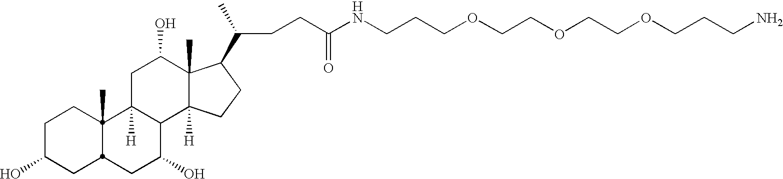

N¹-(Cholic Acid Amide)-4,7,10-trioxo-1,13-tridecanediamine

Cholic acid methyl ester (10 g, 23.66 mmol) and neat 4,7,10-trioxa-1,13-tridecanediamine (130.3 g, 0.592 mol) were combined with 100 mL of absolute ethanol. The mixture was refluxed for 4 days and TLC indicated consumption of cholic acid methyl ester. The solvent and the excess of 4,7,10-trioxa-1,13-tridecanediamine were removed in vacuo. The residue was first dissolved in 50 mL of water and 50 mL of saturated sodium chloride was added. The organic layer was extracted with CH₂Cl₂ (3×200 mL). The combined organic layers were rinsed with saturated salt solution (5×100 mL) and dried over sodium sulfate. Evaporation of organic solvent in vacuo yielded crude product 7.49 g. Crude product was purified to homogeneity by first dissolving in CH₃Cl (150 mL) and stirring with 150 mL of water for 3 hours. The organic layer was dried with sodium sulfate, filtered, and evaporated in vacuo giving 5.81 g (40%). TLC (silica, R_{f}=0.56, i-propanol:30% NH₄OH:H₂O; 10:2:1 v/v/v). ¹H NMR was consistent with the structure. MS for (C₃₄H₆₃N₂O₇) calcd M+1 611.9, found 611.7.

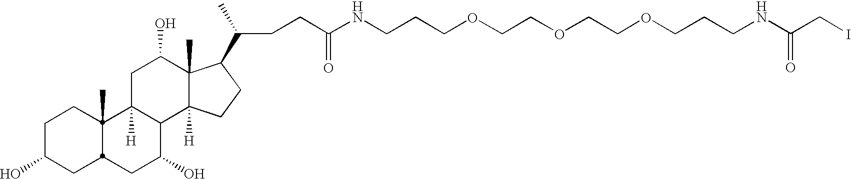

Synthesis of N¹-(Iodoacetamide)-N¹³-(Cholic Acid Amide)-4,7,10-trioxo-1,13-tridecanediamine.

Iodoacetamide was prepared as described for iodoacetamide of chenodeoxy acid derivative above. Yield 860 mg (94%). The product was used for a next step without further purification.

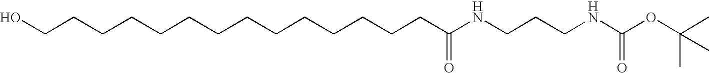

[3-(15-Hydroxy-pentadecanoylamino)-propyl]-carbamic Acid Tert-Butly Ester

ω-Pentadecalactone (26.2 mL, 100 mmol) was added to neat 1,3-diaminopropane (83.50 mL, 1 mol) and the mixture was refluxed overnight. The excess of 1,3-diaminopropane was evaporated in vacuo, the crude intermediate, 15-Hydroxy-pentadecanoic acid (3-amino-propyl)-amide, recrystallized once from methanol, and used without further purification. t-Butyl Pyrocarbonate (6.14 g, 28.1 mmol) was added to the solution of 15-Hydroxy-pentadecanoic acid (3-amino-propyl)-amide (8.03 g, 25.6 mmol) in 200 mL of methanol and stirred overnight at room temperature. The solvent was removed in vacuo and the crude product was purified by flash chromatography (silica, CHCl₃/MeOH, 10:0.8, v/v) to give 8.4 g (79.3%) of the desired product as a white solid. TLC (silica, R_{f}=0.40, CHCl₃/MeOH, 10:1, v/v). ¹HNMR (CDCl₃): δ 1.33-1.51 (br, 33H, (CH₂)₁₂, OC(CH₃)₃), 1.66 (m, 2H, NHCH₂CH₂CH₂NH), 2.22-2.27 (t, 2H, CH₂CONH), 3.22-3.24 (m, 2H, CONHCH₂), 3.35-3.37 (m, 2H, CH₂CH₂NHCOO), 3.70-3.72 (b, 2H, CH₂OH), 5.03 (b, 1H, NH), 6.25 (b, 1H, NH).

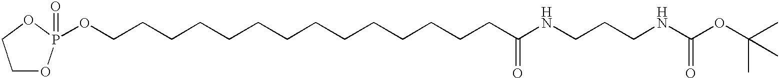

{3-[15-(2-oxo-2λ⁵-[1,3,2dioxaphospholan-2-yloxy)-pentadecanoylamino]-propyl}-carbamic Acid Tert-Butly Ester

First, the white solid of [3-(15-Hydroxy-pentadecanoylamino)-propyl]-carbamic acid tert-butyl ester (0.5 g, 1.21 mmol) was dried in a vacuum oven over P₂O₅ overnight and then was dissolved in 10 mL of freshly distilled THF. Ethylene chlorophosphate (0.26 g, 168 μL, 1.81 mmol) and Et₃N were added to the THF solution of the ester and the mixture was stirred at room temperature under argon overnight. The progress of the reaction was monitored on a TLC by phosphate sensitive molybdenum stain (silica, R_{f}=0.51, CHCl₃/MeOH, 10:1, v/v). The crystalline (Et)₃N.HCl precipitate was removed by filtration, the solvent was removed in vacuo to give the intermediate as a white solid. This reactive intermediate was used immediately for the next step without further purification.

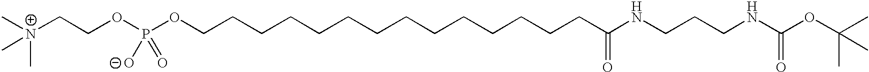

[3-(15-phosphoryl choline-pentadecanoylamino)-propyl]-carbamic Acid Tert-Butly Ester

Trimethylamine (1.5 mL) was added to a Pyrex pressure bottle containing the intermediate from the previous step dissolved in anhydrous acetonitrile (10 mL) and cooled in a dry-ice acetone bath. The pressure bottle was sealed with a teflon stopper and heated at 65° C. for 4 days. The reaction progress was monitored by TLC (silica, R_{f}=0.51, CHCl₃/MeOH, 10:1, v/v) until disappearance of starting material. The solution became cloudy. The solvent was removed in vacuo and 0.6 g of the crude product was purified by flash chromatography (silica, 10 g, MeOH/CHCl₃/H₂O/AcOH, 65:25:4:2, v/v/v/v) to give 0.37 g (52.7%) of a desired product as white solid. The final product was freeze-dried from aqueous solution. TLC (silica, R_{f}=0.21, MeOH/CHCl₃/H₂O/AcOH, 65:25:4:2, v/v/v/v). ¹H NMR (CDCl₃): δ 1.33-1.51 (b m, 33H, (CH₂)₁₂, OC(CH₃)₃), 1.66 (m, 2H, NHCH₂CH₂CH₂NH), 2.22-2.27 (t, 2H, CH₂CONH), 3.22-3.24 (m, 2H, CONHCH₂), 3.35-3.37 (m, 2H, CH₂CH₂NHCOO), 3.46 (bs, 9H, N(CH₃)₃), 3.89-3.91 (b, 4H, CH₂OH, CH₂N(CH₃)₃], 4.45 (b, 2H, CH₂CH₂N(CH₃)₃, 5.03 (b, H, NH), 6.25 (b, H, NH). ESP-MS, M+1 (C₂₈H₅₈O₇N₃P): calculated, 580.40, obtained: 580.6.

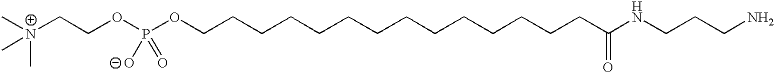

15-phosphoryl Choline-Pentadecanoic Acid (3-amino-propyl)-amide.

The ester from the previous step was deprotected by standard procedure (20 mL, TFA:CH₂Cl₂, 1:1, v/v). The progress of the deprotection was monitored by TLC (silica, R_{f}=0.1, MeOH/CHCl₃/H₂O/AcOH, 65:25:4:2, v/v/v/v) and showed the disappearance of the starting material. The solvent was removed in vacuo, the product dissolved in water, lyophilized, and used in the next step without further purification.

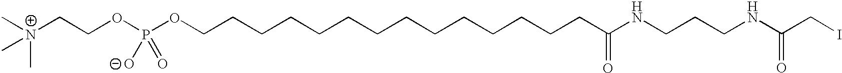

15-phosphoryl Choline-Pentadecanoic Acid (3-iodoacetamide-propyl)-amide

Triethylamine (0.12 g, 0.165 mL, 2.4 mmol) was added to amino amide from the previous step (0.35 g, 0.59 mmol) dissolved in 10 mL of MeOH and was immediately followed by addition of iodoacetyl N-hydroxylsuccinimide (0.34 g, 1.2 mmol). The resulting solution was stirred overnight. The progress of reaction was monitored by TLC (silica, R_{f}=0.21, MeOH/CHCl₃/H₂O/HAc, 65:25:4:2, v/v/v/v). The solvent was removed in vacuo and the crude product was purified by flash chromatography (silica, MeOH/CHCl₃/H₂O/Hac, 65:25:4:2, v/v/v/v) to give 0.29 g (76%) of a desired product as a white solid. The product was dissolved in water and lyophilized. ¹H NMR (CDCl₃): δ 1.33-1.51 (br, 33H, (CH₂)₁₂, OC(CH₃)₃), 1.66 (m, 2H, NHCH₂CH₂CH₂NH), 2.22-2.27 (t, 2H, CH₂CONH), 3.22-3.24 (m, 2H, CONHCH₂), 3.35-3.37 (m, 2H, CH₂CH₂NHCOO), 3.46 (bs, 9H, N(CH₃)₃), 3.89-3.91 (b, 4H, CH₂OH, CH₂N(CH₃)₃), 3.81 (bs, 2H, ICH₂), 4.45 (b, 2H, CH₂CH₂N(CH₃). ESP-MS, M+1 (C₂₅H₅₁IO₆N₃P): calculated, 648.60, obtained: 651.8.

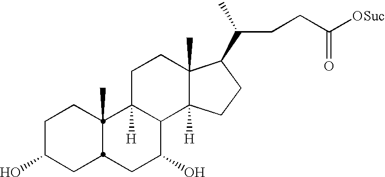

Chenodeoxycholic Acid N-Hydroxysuccinimidyl Ester

N-Hydroxysuccinimide (2.93 g, 25.5 mmol) was added to the solution of Chenodeoxycholic acid (5 g, 12.7 mmol) in 200 mL of freshly distilled THF and stirred for 2.5 hours at room temperature. After 30 minutes of stirring white precipitate of DCU was formed and later removed by filtration. The solution was evaporated in vacuo to give the crude active ester as a white solid. The crude active ester was dissolved in 150 mL of CHCl₃ and washed with saline/brine solution (0.1 N Na₂CO₃, 5 M NaCl, 3×150 mL). The organic layer was separated and dried over anhydrous Na₂SO₄. The solvent was removed in vacuo to give 5.85 g (11.9 mmol, 93.7%) of desired product as a white solid. TLC (silica, R_{f}=0.71, CHCl₃/MeOH, 10:2 v/v). ¹H NMR (CDCl₃): δ 0.68 (b, 3H, CH₃), 0.90-2.75 (b, 34H), 2.87 (bs, 4H, COCH₂CH₂), 3.50 (b, 2H, CHOH), 3.78 (b, H, CHOH), 3.88 (b, H, CHOH).

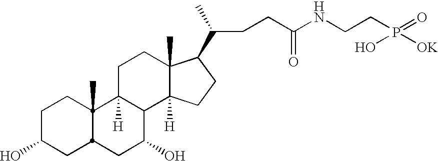

N-Chenodeoxycholyl-2-aminoethyl-phosphonic Acid Monopotassium Salt

2-Aminoethylphosphonic acid (0.30 g, 2.4 mmol) was dissolved in 5 mL of aqueous solution of potassium hydroxide (0.27 g, 4.8 mmol) and lyophilized to obtain 2-aminoethylphosphonic acid dipotassium salt as a colorless glass. This dipotassium salt was dissolved in 20 mL of MeOH and added to the 20 mL of methanolic solution of Chenodeoxycholic Acid N-Hydroxysuccinimidyl ester (1.41 g, 2.88 mmol). The resulting clear solution was stirred at room temperature overnight. Some unreacted 2-aminoethylphosphonic acid dipotassium salt that had precipitated was removed by filtration. The solvent was removed in vacuo and the crude product was redissolved in water. At this step, unreacted Chenodeoxycholic acid N-Hydroxysuccinimidyl ester was removed by filtration. The volume was reduced in vacuo and the product was lyophilized from water giving 1.66 g of crude product as a colorless glass. The crude product was first purified by flash chromatography (15 g of freshly baked SiO₂, isopropanol/AcOH/H₂O, 100:14:12 v/v/v) to give 0.87 g of glassy white solid. TLC indicated that this solid still contained some N-Hydroxysuccinimide. The solid (0.87 g) was divided into four batches of approximately equal weight (˜200 mg) and each batch was further purified with prepacked Amprep C18 column (500 mg sorbent per column). Each batch of solid dissolved in 0.5 mL of water was loaded on the column. The column was eluted first with 4 mL of water then with 4 mL of ethanol. The fractions containing the product were combined. The solvents were removed in vacuo and the final product was freeze-dried from water to give a white solid. Overall yield of the desired product was 0.24 g (18.6%). TLC (silica, R_{f}=0.43, iso-Propanol/AcOH/H2O 100:14:12 v/v/v). ¹HNMR(D₂O): δ 0.68 (b, 3H, CH₃), 0.90-2.75 (b, 36H), 3.37-3.40 (b, 2H, CH₂NH), 3.50 (b, H, CHOH), 3.90 (b, H, CHOH). ESP-MS of M+1 (C₂₆H₄₅O—₆NPK): calculated, 538.71, obtained, 538.4.

Poly-L-lysine-graft-R₁-graft-R₂-graft-R₃ Co-Polymers

A variety of poly-L-lysine-graft-copolymers was successfully synthesized through epoxide, tosyl, vinyl sulfone and haloacetamido chemistries. These chemistries were selected over typical activated ester approach to preserve charges on polycation and minimize impact on conjugate-DNA binding. These copolymers, poly-L-lysine-graft-R₁-graft-R₂-graft-R₃ co-polymers, could have a variety of molecules grafted on amino groups of cationic poly-L-lysine in a stepwise synthesis. Typically, PEG molecules are grafted first (R₁), followed by introduction of other molecules (R₂), and finally fluorescent tags or ligand molecules (R₃), are covalently attached to some copolymers. The synthesis of these copolymers is described below.

Poly-L-lysine-graft-R₁-graft-R₂-graft-R₃ Co-Polymers (R₁=PEG Derivative; R₂=None, R₃=None)

Poly-L-lysine-graft-PEG polymers were prepared by reacting a PEG-electrophile with ε-NH₂ lysine groups under basic conditions. For individual co-polymers, the ratios of PEG-electrophile to poly-L-lysine, PEG-electrophile size, and poly-L-lysine size were varied. The conditions of the syntheses are summarized in Table 2 and the general procedure is described for PEG-epoxide below.

Poly-L-lysine 10K (600 mg, 0.06 mmol) and lithium hydroxide monohydrate (41 mg, 2.9 mmol) were dissolved in water (2 ml) and methanol (6 ml) in a siliconized glass flask. Solid PEG5K-epoxide (600 mg, 0.12 mmol) was added, the flask was then sealed, and the solution incubated at 65° C. for 48 h. After incubation, the solvent was removed in vacuo. The product was redissolved in a loading buffer (0.1 M sodium phosphate pH 6 in 10% MeOH (v/v)) and loaded on cation exchange column (Amersham Pharmacia SP Sepharose FF resin) followed by extensive washing step (up to 10 column volumes). The product then was eluted with 0.1 N NaOH in 10% MeOH solution. The macromolecular fractions containing the product were combined and the solvent removed in vacuo. The product containing inorganic salts was re-dissolved in minimum amount of 0.05 M acetic acid in 30% MeOH solution and eluted over a G-25 column (Amersham Pharmacia Sephadex G-25 fine resin) with the same acetic acid solution. The macromolecular fractions were pooled and lyophilized. The ratio of PEG chains to poly-L-lysine chains was determined by ¹H NMR.

TABLE 2
PEG Grafts (Poly-L-lysine-graft-R₁-graft-R₂-graft-R₃ co-polymers; R₁ = PEG; R₂ = None; R₃ = None)

| | | | | | | | Effective | |
|---|---|---|---|---|---|---|---|---|
| | | Graft | Stoichio- | | | | Diameter | Effective |
| | Type of | Size × | metric | ¹NMR Ratio | PLL Size | PLL | in H₂O | Diameter in |
| ID | Graft (R₁) | 10³ | Ratio | R₁/PLL | ×10³ | Dₚ | [nm] | NaCl [nm] |
| PG-A | PEG-Tosyl | 2 | 20 | 25 | 9.6 | 46 | * | ND |
| PG-B | PEG-Tosyl | 2 | 12.5 | 44 | 9.6 | 46 | * | ND |
| PG-C | PEG-Tosyl | 2 | 25 | 44 | 9.6 | 46 | * | ND |
| PG-D | PEG-Epoxide | 3 | 2 | 5 | 9.4 | 45 | 97.3 (2.5) | 99.8 |
| PG-E | PEG-Epoxide | 3 | 5 | 9.7 | 9.4 | 45 | 124.1 | 115.4 |
| PG-F | PEG-Epoxide | 3 | 10 | 11.9 | 9.4 | 45 | 88 | 88.3 |
| PG-G | PEG-Epoxide | 5 | 5 | 10.6 | 9.4 | 45 | 114.3 | 110.6 |
| PG-H | PEG-Epoxide | 3 | 10 | 16.6 | 9.4 | 45 | 118.1 | 115.5 |
| PG-I | PEG-Vinyl | 5 | 5 | 1 | 47Cys | 47 | 82 | ND |
| | sulfone | | | | | | | |
| PG-J | PEG-Vinyl | 5 | 5 | 1 | 47Cys | 47 | 94.2 | 83.5 |
| | sulfone | | | | | | | |
| PG-K | PEG-Epoxide | 5 | 2 | 4.5 | 10 | 48 | 72.8 | ND |
| PG-L | PEG-Epoxide | 5 | 5 | 10.4 | 10 | 48 | 83.0 | ND |
| PG-M | PEG-Epoxide | 5 | 36 | 38 | 10 | 48 | 153.0 | 147.2 |
| PG-N | PEG-Epoxide | 5 | 2 | 3.2 | 10 | 48 | 67.7 | 93.1 |
| PG-O | PEG-Epoxide | 5 | 5 | 7.5 | 10 | 48 | 111.4 | 166.3 |
| PG-P | PEG-Epoxide | 5 | 10 | 14.3 | 10 | 48 | 125.1 | ND |
| PG-Q | PEG-Epoxide | 5 | 2 | 2 | 9.4 | 45 | 116.8 | 111.0 |
| PG-R | PEG-Epoxide | 5 | 10 | 12.9 | 9.4 | 45 | 167.0 | 122.4 |
| PG-S | Br—CH₂—(CO)—NH— | 3.4 | 25 | 33 | 26 | 125 | * | ND |
| | PEG-COOH | | | | | | | |
| PG-T | Br—CH2—(CO)—NH— | 3.4 | 30 | 37 | 26 | 125 | 83.3 | ND |
| | PEG-NH-t-BOC | | | | | | | |
| PG-U | PEG-Epoxide | 3 | 15 | 16 | 26 | 126 | * | ND |
| PG-V | PEG-Epoxide | 3 | 75 | 87 | 26 | 126 | * | ND |
| PG-W | PEG-Epoxide | 3 | 25 | 22 | 26 | 126 | * | ND |
| PG-X | PEG-Epoxide | 5 | 2 | 2.4 | 26 | 125 | 54.0 | 65.7 |
| PG-Y | PEG-Epoxide | 5 | 5 | 6 | 26 | 125 | 61.9 | 67.9 |
| PG-Z | PEG-Epoxide | 5 | 10 | 11.1 | 26 | 125 | 88.6 | 72.3 |
| PG-AA | Branched PEG- | 10 | 15 | 11.9 | 26 | 125 | * | ND |
| | (CO)—NH—(CH₂)₃— | | | | | | | |
| | Br | | | | | | | |
| PG-AB | PEG-Epoxide | 5 | 2 | 2.5 | 38 | 181 | 76.6 | ND |
| PG-AC | PEG-Epoxide | 5 | 5 | 6 | 38 | 181 | 84.7 | ND |
| PG-AD | PEG-Epoxide | 5 | 10 | 11.7 | 38 | 181 | 136.0 | ND |
| PG-AE | PEG-Epoxide | 5 | 30 | NA | PEI 25 | 583 | * | ND |
| PG-AF | PEG-Epoxide | 5 | 813 | NA | PEI 70 | 1632 | * | ND |
| PG-AG | PEG-Epoxide | 5 | 872 | NA | PEI 750 | 14483 | * | ND |

*Count rate too low to measure size by LLS..
NA—Not Applicable.
ND—Not Done

Poly-L-lysine-graft-R₁-graft-R₂-graft-R₃ Co-Polymers (R₁=PEG; R₂=Hydrophobe; R₃=None)

The hydrophobically modified series of poly-L-lysine-graft-R₁-graft-R₂ co-polymers was synthesized through epoxide, bromoalkyl and amidine chemistry. The products of such syntheses are listed in Tables 2 and 3. The exemplary synthesis is described below.

Poly-L-lysine 10k-graft-(ε-NH-PEG5k)_{14.3} (100 mg, 1.2 mmol) and lithium hydroxide (1.7 mg, 41 mmol) were dissolved in methanol (10 ml) in a siliconized flask. To this solution, dodecyl/tetradecyl glycidyl ether (8.9 mg, 36.8 mmol) was added. The flask was capped tightly and incubated at 65° C. After 48 h incubation, the methanol was removed in vacuo, the residue was re-dissolved in water (4 ml), and the pH was adjusted to 4 with glacial acetic acid. The mixture was eluted over G-25 column with 0.01 M acetic acid. The ratio of dodecyl/hexadecyl chains grafted to PEG-PL was determined by 300 MHz ¹H NMR.

Poly-L-lysine-graft-(ε-NH-PEG5k)_{12.8}-graft-(ε-NH—CH₂CONHCH₂CH₂CH₂-O-β-cholesterol ether)₂₆.

Poly-L-lysine 9.4K (200 mg, 0.02 mmol) and lithium hydroxide monohydrate (15.1 mg, 0.36 mmol) were dissolved in methanol (15 mL) and water (1.0 mL) in a siliconized glass flask. Then solid PEG5K-epoxide (900 mg, 0.18 mmol) was added. The flask was then sealed and incubated at 65° C. for overnight. Then BrCH₂CONH(CH₂)₃O-β-Cholesteryl ether (254 mg, 0.45 mmol) was added and the resulting solution was incubated at 65° C. over night. After incubation, the solvent was removed in vacuo and the product was re-dissolved in a minimum amount of water. The pH of the solution was adjusted to pH 4 with glacial acetic acid. The resulting solution was eluted over a G-25 column (Amersham Pharmacia Sephadex G-25 fine resin) with 0.1 M of acetic acid. The macromolecular fractions were pooled and lyophilized. The ratio of PEG and cholesteryl moieties to poly-L-Lysine was determined by ¹H NMR and is presented in Table 3.

Poly-L-lysine26k-graft-(ε-NH-PEG5k)_{12.2}graft-(ε-NH—(CH₂)₁₀—N-pyridine_{20.8}

Lithium hydroxide monohydrate (30.6 mg, 0.73 mmol) dissolved in water (1 mL) was added to a solution of poly-L-Lysine 26K (257.3 mg, 0.0099 mmol) in 10 mL of MeOH. PEG5K-epoxide (495 mg, 0.099 mmol) was then added. The flask was sealed and the solution incubated at 65° C. for 8 hours. Then Br(CH₂)₁₀—N-pyridinuim bromide (300 mg, 0.79 mmol) was added and the resulting solution was incubated at 65° C. for 3 days. After incubation, the solvent was removed in vacuo and the product was re-dissolved in a minimum amount of water. The pH of the solution was adjusted to pH 4 with glacial acetic acid. The resulting solution was eluted over a G-25 column (Amersham Pharmacia Sephadex G-25 fine resin) with 0.1 M of acetic acid. The macromolecular fractions were pooled and lyophilized yielding 486 mg.

TABLE 3.
Hydrophobic Groups Grafted on a PLL Chain.

| | | | | | | ¹H | | | Eff. | |
|---|---|---|---|---|---|---|---|---|---|---|
| | Type of | Stoichio- | ¹H NMR | | Stoichio- | NMR | PLL | | Dia. in | Eff. Dia. |
| | Graft | metric | Ratio | Type of | metric | Ratio | Size | PLL | H₂O | in NaCl |
| ID.: | (R₁) | Ratio | R/PLL | Graft (R₂) | Ratio | R₂/PLL | ×10³ | Dp | [nm] | [nm] |
| HG-A | PEG- | 5 | 5 | C₁₂- | 30 | 29.2 | 10 | 48 | 155.9 | 138.8 |
| | epoxide 3 k | | | Epoxide | | | | | | |
| HG-B | PEG- | 15 | 20 | C₁₂/C₁₄ glyc- | 30 | 30.5 | 10 | 48 | 99.0 | ND |
| | epoxide 5 k | | | idyl ether | | | | | | |
| HG-C | None | NA | NA | C₁₂-Epoxide | 15 | 27.5 | 9.4 | 45 | 70.7 | ND |
| HG-D | PEG- | 2 | 3 | C₁₂- | 30 | 87.5 | 10 | 48 | 117.0 | 117.0 |
| | epoxide 5 k | | | Epoxide | | | | | | |
| HG-E | None | NA | NA | C₁₂- | 41 | 81 | 26 | 125 | 63.0 | 115.6 |
| | | | | Epoxide | | | | | | |
| HG-F | PEG- | 25 | 19 | C₁₂- | 98 | 78 | 26 | 126 | 81.9 | 89.1 |
| | epoxide 3 k | | | Epoxide | | | | | | |
| HG-G | PEG- | 10 | 6.8 | C₁₂- | 41 | 48 | 26 | 125 | Not | ND |
| | epoxide 5 k | | | Epoxide | | | | | Soluble | |
| HG-H | None | NA | NA | C₁₈—Br | 123 | 64 | 26 | 123.8 | 60.6 | ND |
| HG-I | None | NA | NA | C₁₈—Br | 25 | 22.8 | 26 | 123.8 | 1318.3 | ND |
| HG-J | PEG- | 12 | 8.8 | C₁₈—Br | 123 | 85.4 | 26 | 123.8 | ND | ND |
| | epoxide 5 k | | | | | | | | | |
| HG-K | None | NA | NA | C₁₂ | 20 | 20 | PEI 25 | 583 | ND | ND |
| | | | | Epoxide | | | | | | |
| HG-L | None | NA | NA | Br—C₁₂— | 26 | 16 | 9.6 | 46 | ND | ND |
| | | | | COO- | | | | | | |
| HG-M | None | NA | NA | C₇—COO⁻ | XS | 123.8 | 26 | 123.8 | 46.3 | 805.9 |
| HG-N | PEG- | 7 | 11 | Br—CH₂— | 50 | 49.2 | 9.4 | 45 | 382.9 | 377.0 |
| | epoxide 5 k | | | (CO)— | | | | | | |
| | | | | N(C₁₂)₂ | | | | | | |
| HG-O | PEG- | 10 | 12.2 | N- | 80 | 20.8 | 26 | 123.8 | ND | ND |
| | epoxide 5 k | | | pyridinium- | | | | | | |
| | | | | C₁₀—-Br | | | | | | |
| | | | | Bromide Salt | | | | | | |

NA—Not Applicable.
ND—Not Done.

Some grafting elements were attached to cationic polymer by amidine functionality. Such grafting elements were prepared starting with cyano derivatives that were transformed into imino methyl esters in a presence of HCl and anhydrous methanol in CH₂Cl₂ and as shown on Scheme 4 and 5. The polymeric products are described in Table 4.

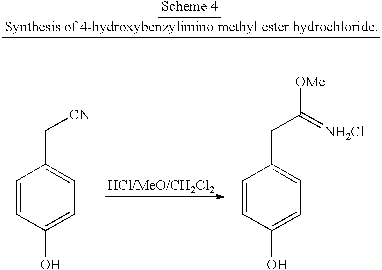

4-hydroxybenzylimino Methyl Ester Hydrochloride.

4-hydroxybenzyl cyanide 0.7354 g (5.52 mmol) was dissolved in 20 mL of CH₂Cl₂, 800 μL of anhydrous methanol was added and the reaction was cooled to 0° C. The reaction mixture was saturated with HCl gas and maintained 0° C. The resulting white precipitate was collected, washed thoroughly with cold dichloromethane and anhydrous diethyl ether and dried in vacuo over to yield 1.084 g (97%). The imino methyl ester was used in the following step without further purification.

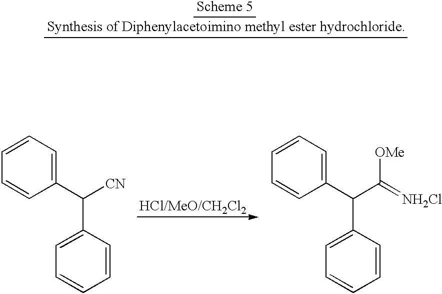

Diphenylacetoimino Methyl Ester Hydrochloride.

Diphenylacetonitrile (9.65 g, 49.9 mmol) was dissolved under argon in dichloromethane (60 mL), methanol (6.52 mL, 165 mmol) was added, and the reaction mixture was cooled to 0° C. The reaction mixture was saturated with HCl gas and maintained at 0° C. overnight. The final product was then precipitated in ethyl ether to yield 9.0 g (70%) as a white solid. The product was used for the next step without further purification.

Synthesis of PL10k-graft-(ε-NH-(4-Hydroxybenzyl)amidine) (HG-Q)

PL10k-graft-(PEG5k)_{7.9}-graft-(NH—C(═NH)-benzyl)_{5.5}

PL10k-graft-(PEG5k)_{7.9} (200 mg, 0.004 mmol) and the hydroxybenzylimino methyl ester hydrochloride (183.7 mg, 0.911 mmol) were dissolved in a 4 ml mixture of methanol and water (1:1 v/v). The pH was adjusted to 10.9 with 10 N NaOH, and the solution was stirred under argon at ambient temperature. After 16 h, the solution was evaporated in vacuo and the residue was re-dissolved in 0.05 N acetic acid. The solution was purified on a G-25 size exclusion column (30×4 cm column, 0.05 N acetic acid). The macromolecular fractions were pooled and lyophilized to yield 156.7 mg (75%) of product. ¹H NMR (D₂O) indicated 7.9 PEG chains and 5.5 hydroxyphenyl chains per PL chain.

TABLE 4
Hydrophobic Groups Grafted via Amidine Chemistry

| | | | | | | | | | Effective |
|---|---|---|---|---|---|---|---|---|---|
| | | Stoichio- | ¹NMR | | Sotto- | ¹NMR | PLL | Effective | Diameter |
| | Type of | metric | Ratio | Type of Graft | metric | Ratio | Dp | Diameter in | in NaCl |
| ID | Graft (R₁) | Ratio | R₁/PLL | (R₂) | Ratio | R₁/PLL | ×10³ | H2O [nm] | [nm] |
| HG-P | None | NA | NA | Hydroxybenzyl- | XS | 5.2 | 48 | 56.3 | ND |
| | | | | amidine | | | | | |
| HG-Q | PEG- | 5 | 7 | Hydroxybenzyl- | 205 | 5.5 | 48 | 87.6 | ND |
| | Epoxide 5 k | | | amidine | | | | | |
| HG-R | PEG- | 40 | 38 | Hydroxybenzyl- | 400 | 3.7 | 48 | 70.6 | ND |
| | Epoxide 5 k | | | amidine | | | | | |
| HG-S | PEG- | 5 | 7 | Hydroxybenzyl- | 99 | 27 | 48 | 112.4 | ND |
| | Epoxide 5 k | | | amidine | | | | | |
| HG-T | PEG- | 10 | 15.2 | Hydroxybenzyl- | XS | 32.5 | 48 | ND | ND |
| | Epoxide 5 k | | | amidine | | | | | |
| HG-U | PEG- | 5 | 11 | Biphenyl | XS | 99 | 125 | ND | ND |
| | Epoxide 5 k | | | | | | | | |

Hydroxybenzyl-amidme MW 201;
Biphenyl MW 227.
NA—Not Applicable.
XS—Excess

Poly-L-lysine-graft-R₁-graft-R₂-graft-R₃ Co-Polymers (R₁=None or PEG; R₂=None; R₃=Ligand, Endosomal Escape Moiety or Fluorescent Probe)

Ligands and endosomal escape molecules along with fluorescent labels were grafted on amino groups of co-polymers in a final step. The ligand molecules were grafted via bromoacetyl chemistry. The picolyl moieties were introduced onto co-polymers using picolyl chloride. The products were purified and characterized as described earlier. The products containing grafted ligands and endosomal escape molecules are listed on Table 5. Fluorescently labeled conjugates were prepared following manufacturer protocol and are listed in Table 6. These fluorescently labeled conjugates were used to evaluate polyplex interactions in vivo and in vitro with hepatocytes. Their uptake was also investigated by histological methods.

PL10k-graft-(ε-NH—(CH₂)₁₀PEG2k)₄-graft-(ε-NH-(4-picolyl))₃₀ (1408:022)

PL10k-graft-(ε-NH—(CH₂)₁₀PEG2k)₄ (PL-E) (115 mg, 0.0062 mmol) and 4-Picolyl Chloride (49 mg, 0.301 mmol) were dissolved in methanol (8 ml). Lithium Hydroxide (22 mg, 0.54 mmol) was added as a solution in methanol (1 mL). The reaction was incubated at 65° C. for 6 days. The reaction mixture was then evaporated to dryness. It was re-dissolved in 0.05 N acetic acid in 30% methanol, and chromatographed over G-25 column. The macromolecular fraction was collected, and evaporated to dryness to yield 84 mg of red-brown solid. ¹H NMR (D₂O): δ 8.66 (br-m, Ar—H); 8.03 (br-m, Ar—H); 4.15 (br-s, Cα-H), 3.56 (s, O—CH₂—CH₂), 3.27 (s, PEG-O—CH₃); 2.82 (s, ε-CH₂); 1.19 (br-s, (CH₂)₃). Ratio of PLL:PEG:Picoline was 1:4:30 as determined by ¹H NMR.

TABLE 5
Ligand Containing Grafts.

| | | | ¹H | | | | | | Effective | |
|---|---|---|---|---|---|---|---|---|---|---|
| | Type of | Stoichio- | NMR | Type of | Stoichio- | ¹H NMR | PLL | | Dia in | Effective |
| | Graft | metric | Ratio | Graft | metric | Ratio | Size | PLL | H₂O | Diameter in |
| ID.: | (R₁) | Ratio | R/PLL | (R₂) | Ratio | R₂/PLL | ×10³ | Dp | [nm] | NaCl [nm] |
| LG-A | None | NA | NA | Cholic Acid | 123 | 123.8 | 26 | 123.4 | 202.4 | 680.4 |
| | | | | Derivative | | | | | | |
| LG-B | PEG- | 9 | 12.8 | Cholesterol | 22.5 | 26 | 9.4 | 45 | 310.0 | 296.9 |
| | Epoxide 5 k | | | Derivative | | | | | | |
| LG-C | None | NA | NA | Cholesterol | 14 | 17.8 | 9.4 | 45 | 336.9 | 781.2 |
| | | | | Derivative | | | | | | |
| LG-D | None | NA | NA | Trigal- | 10 | 16.1 | 9.4 | 45 | ND | ND |
| | | | | NH(CO)CH 2Br | | | | | | |
| LG-E | PEG2k- | 40 | 4.7 | Trigal- | 5 | 9 | 9.4 | 45 | ND | ND |
| | (CH₂)₁₀Br | | | NH(CO)CH | | | | | | |
| | | | | 2Br | | | | | | |
| LG-F | PEG- | 10 | 12.6 | Lactose- | 10 | 8.8 | 9.6 | 45 | ND | ND |
| | Epoxide 3 k | | | (CO)-C12- | | | | | | |
| | | | | Br | | | | | | |
| LG-G | PEG2k- | 40 | 4 | Picolyl-Cl | 48 | 30 | 9.4 | 45 | ND | ND |
| | (CH₂)₁₀Br | | | | | | | | | |
| LG-H | None | NA | NA | Chenodeoxy | 0.731 | 10 | 9.4 | 45 | 379.9 | 553.4 |
| | | | | cholic Acid- | | | | | | |
| | | | | Br | | | | | | |
| LG-I | None | NA | NA | Chenodeoxy | 0.731 | 10 | 9.4 | 45 | ND | ND |
| | | | | cholic Acid- | | | | | | |
| | | | | Br | | | | | | |
| LG-J | None | NA | NA | Cholic | 0.747 | 10 | 9.4 | 45 | ND | ND |
| | | | | Acid-Br | | | | | | |
| LG-K | None | NA | NA | Chenodeoxy | 0.731 | 10 | 9.4 | 45 | ND | ND |
| | | | | cholic Acid- | | | | | | |
| | | | | Br | | | | | | |
| LG-L | None | NA | NA | Chenodeoxy | 0.731 | 10 | 9.4 | 120 | ND | ND |
| | | | | cholic Acid- | | | | | | |
| | | | | Br | | | | | | |
| LG- | PEG2k- | 36 | 9 | Chenodeoxy | 0.731 | 10 | 9.4 | 45 | ND | ND |
| M | (CH₂)₁₀Br | | | cholic Acid- | | | | | | |
| | | | | Br | | | | | | |
| LG-N | PEG- | 5 | 6 | Chenodeoxy | 0.731 | 10 | 7 | 45 | ND | ND |
| | Epoxide 5 k | | | cholic Acid- | | | | | | |
| | | | | Br | | | | | | |
| LG-O | PEG- | 5 | 2 | Chenodeoxy | 0.731 | 10 | 4 | 45 | ND | ND |
| | Epoxide 5 k | | | cholic Acid- | | | | | | |
| | | | | Br | | | | | | |

NA—Not Applicable.
ND—Not Done.
PEG2k-(CH₂)₁₀Br ( )

Poly-L-lysine-graft-R₁-graft-R₂-graft-R₃ Co-Polymers (R₁=None or PEG; R₂=None; R₃=Ligand, Endosomal Escape Moiety or Fluorescent Probe)

Ligands and endosomal escape molecules along with fluorescent labels were grafted on amino groups of co-polymers in a final step. The ligand molecules were grafted via bromoacetyl chemistry. The picolyl moieties were introduced onto co-polymers using picolyl chloride. The products were purified and characterized as described earlier. The products containing grafted ligands and endosomal escape molecules are listed in Table 5. Fluorescently labeled conjugates were prepared following manufacturer protocol and are listed in Table 6. These fluorescently labeled conjugates were used to evaluate polyplex interactions in vivo and in vitro with hepatocytes. Their uptake was also investigated by histological methods.

Poly-L-lysine9.6k-graft-(ε-NH-PEG3k)_{12.6}-graft-(ε-NH—(CH₂)₁₀—CO—NH-Lactose)_{8.8}

Poly-L-lysine 9.4K (150 mg, 0.016 mmol) and lithium hydroxide monohydrate (18.1 mg, 0.43 mmol) were dissolved in a mixture of methanol (10 mL) and water (0.5 mL) in a siliconized glass flask. Solid PEG3K-epoxide (478.7 mg, 0.16 mmol) was added. The flask was then sealed and the clear solution incubated at 65° C. for 2 days. After 6% TBU gel showed the disappearance of free PL9.4K, Br(CH₂)₁₀—CO-lactosylamide was added in MeOH (3 mL) and the resulting solution was incubated at 33° C. for 2 days. After incubation, the solvent was removed in vacuo and the product was re-dissolved in a minimum amount of water. The pH of the solution was adjusted to pH 4 with glacial acetic acid. The product was eluted over a G-25 column (Amersham Pharmacia Sephadex G-25 fine resin) with 0.1 M of acetic acid. The macromolecular fractions were pooled and lyophilized. The desired product was characterized by ¹HNMR and the ratio of lactose residues reported in Table 5.

Some polymers were fluorescently labeled for mechanistic studies. The synthesis of such fluorescent conjugates is described briefly below. The conjugates prepared are listed in Table 6.

PL10K/PEG5K/Cy5 (FL-B)

Sodium bicarbonate (15 mg, 0.18 mmol) and sodium carbonate (5 mg, 0.047 mmol) were dissolved in 20 mL of H₂O to give a 1.0 mg/mL carbonate buffer at pH 9.47. Then, a vial of Cy5 (100 mmol) was added to the polymer solution in carbonate buffer (16.4 mg, 365 mmol, 1 mL). The reaction mixture was kept in the dark overnight. Then, the solvent was removed in vacuo and the blue product was purified on PD-10 column (Sephadex G-25, acetic acid 0.05 M). Fractions containing blue conjugate were pooled and lyophilized to give 6 mg of blue solid. The fluorescently labeled product was used without further purification.

TABLE 6
Fluorescent Labeled Conjugates

| | Type of | Graft | Stoichio- | ¹H NMR | Type of | | ¹H NMR | PLL | |
|---|---|---|---|---|---|---|---|---|---|
| IRC | Graft | Size | metric | Ratio | Graft | Stoichiometric | Ratio | Size | |
| Lot No.: | (R₁) | ×10³ | Ratio | R/PLL | (R₂) | Ratio | R₂/PLL | ×10³ | PLL Dp |
| FL-A | None | NA | NA | NA | CY5 | 0.1 | NA | 10 | 48 |
| FL-B | PEG- | 5 | 7 | 7 | CY5 | 0.14 | NA | 10 | 48 |
| | Epoxide | | | | | | | | |
| FL-C | PEG- | 5 | 10 | 14.3 | CY5 | 0.5 | NA | 10 | 48 |
| | Epoxide | | | | | | | | |
| FL-D | PEG- | 2 | 40 | 12 | CY5 | 0.23 | NA | 9.4 | 45 |
| | C10-Br | | | | | | | | |
| FL-E | None | NA | NA | NA | CY5 | 0.19 | NA | 10 | 48 |

NA—Not Applicable. Reference: Product specification, FluoroLink ™ Cy5 reactive dye 5-pack, PA 25000, Amersham Pharmacia Biotech Inc.

### Example 2

### Random Grafts of PEG Coupled Hydrophobic Molecules on PLL Chains

Materials and Methods

Poly-L-lysine (PLL) 10K [DP (Vis) 48, MW (Vis) 10,000; DP (LALLS) 32, MW (LALLS) 6,700, Mw/Mn (SEC-LALLS) 1.20], 26K [DP (Vis) 123, MW (Vis) 25,700; DP (LALLS) 120, Mw (LALLS) 25,000, Mw/Mn (SEC-LALLS) 1.20], 38K [DP(Vis) 184, Mw (Vis) 38,500; DP (LALLS) 172, Mw (LALLS) 35,900; Mw/Mn (SEC-LALLS) 1.10], Poly-L-aspartic acid (P(Asp)) sodium salt 10K [DP (Vis) 76, Mw 10,400 (Vis); DP (LALLS) 57, Mw (LALLS) 7,800] and ethidium bromide were purchased from Sigma Chemical Co., St. Louis, Mo. Tris (2-carboxyethyl) phosphine hydrochloride (TCEP.HCL) was purchased from Pierce Chemical Co. (Rockford, Ill.). PD 10 Sepadex G-25M (pre-packed) and phenyl sepharose high performance (hydrophobic interaction column [HIC]) columns and G-25M resin were purchased from Pharmacia Biotech Inc. (Piscataway, N.J.). The CM/M Poros column (CM) was purchased from PerSeptive Biosystems, Inc. (Farmington, Mass.). Synthetic polylysine, (Lys)₄₈Cys, was purchased from Dr. Schwabe (Protein Chemistry Facility at the Medical University of South Carolina). Polyethylene glycol (PEG) epoxides 2K (Mₙ 1554; M_{w}/Mₙ 1.044 (GPC)), 3K (Mₙ 2696; M_{w}/Mₙ 1.035 (GPC)), and 5K (Mₙ 5231; M_{w}/Mₙ 1.017 (GPC)) were purchased from ShearWater Polymers, Inc. (Huntsville, Ala.). LiOH.H₂O was purchased from Aldrich Chemical Co. (Milwaukee, Wis.). Plasmid DNA (pCMVβ, Clontech, Palo Alto, Calif. and pCMV-Luciferase was prepared by BIO 101 (San Diego, Calif.). Plasmid DNA preparation contained more than 90% covalently closed circular DNA as determined by agarose gel electrophoresis.

Synthesis of Grafting Elements

Synthesis, purification, and characterization of grafting elements are described in subsequent parts. The synthesis of grafting element with hydrophobic domain is illustrated on Scheme 6.

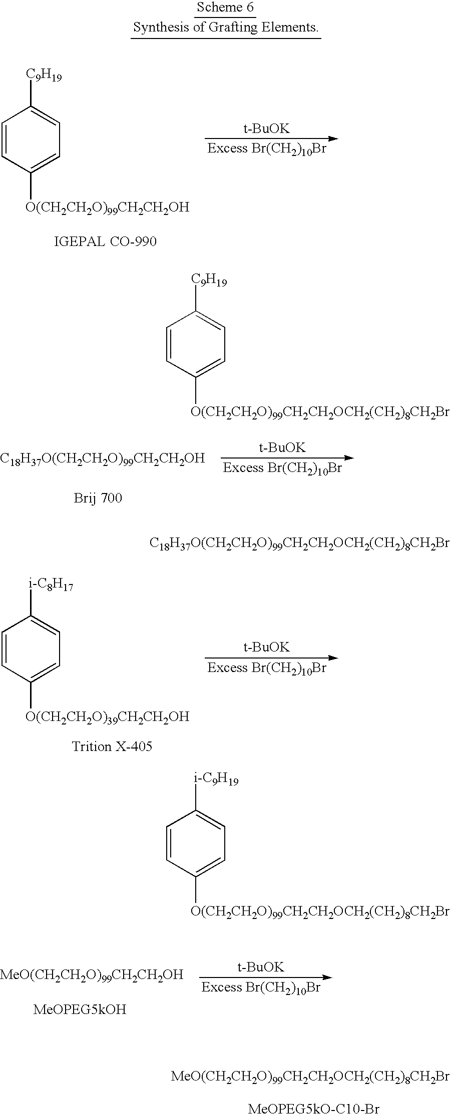

α-Methoxy-ω-(O-11-bromoundecan-1-oxy)-poly(ethylene oxide 2k)

A solution of 40 grams (40 mmol) of α-Methoxy-ω-hydroxy-poly(ethylene oxide) 2k in 200 mL of toluene was refluxed and water removed as an azeotrope using Dean-Stark distillation head. The solution was cooled to approximately 30° C. and solid potassium t-butoxide was added. The resulting suspension was stirred at room temperature until complete dissolution of base and was followed by the addition of solid 1,10-dibromodecane. The mixture was gently heated and stirred for three days. 3 mL of glacial acetic acid was added to neutralize excess base. The solids were removed by filtration, the volume reduced in vacuo, and the product precipitated in a large excess of anhydrous diethyl ether (500 mL). The product was recrystallized from methylene chloride/ethyl ether to yield 42.8 grams (96%). The purity of the name product was determined by ¹H NMR to be approximately 72%. The crude product contained approximately 28% of α-methoxy-ω-(O-11-undec-1-ene)-poly(ethylene oxide 2k) formed as bromide elimination side product. This unsaturated derivative of PEG did not interfere with the next step and was removed at the final purification step. ¹H NMR (CDCl₃) d 3.62 (PEG,), 3.54 (m, CH₂Br), 3.40 (m, CH₂), 3.38 (s, CH₃O,) 1.82 (m, CH₂), 1.53 (m, CH₂), 1.26 (m, CH₂).

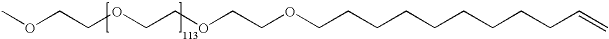

α-methoxy-ω-(O-11-undecene-1)-poly(ethylene oxide 5k)

Potassium t-Butoxide (1.13 g, 10.1 mmol) was added to Poly(ethylene glycol)monomethyl ether 5k (25.25 g, 5.05 mmol) that was azeotropically dried using toluene (200 mL). The solvents were removed in vacuo resulting in a yellow viscous residue. The residue was dissolved in freshly distilled THF (200 mL) and immediately followed by addition of 11-Bromo-1-Undecene (2.36 g, 2.22 mL, 10.1 mmol). First, the resulting solution was stirred at room temperature under Argon and in the dark for two days, and then, it was briefly refluxed for 2 hours resulting in a cloudy solution. The solvent was evaporated in vacuo and the yellow solid was dissolved in a minimum amount of chloroform and precipitated into anhydrous ethyl ether. The solid was collected by centrifugation and rinsed three times with ethyl ether. The desired product was dried in vacuum oven overnight and obtained as a white solid (17.05 g, 3.31 mmol, 65.5%). ¹H NMR (CDCl₃): δ 1.30-1.35 (b, 10H, (CH₂)₅), 1.55-1.65 (b, 2H, CH₂), 2.00-2.20 (b, 4H, 2×(CH₂)), 3.50-4.00 (b, 459H, (CH₂CH₂O)₁₁₃+OCH₂+CH₂CH═CH₂+OCH₃), 5.00-5.15 (b, 2H, CH═CH₂), 5.90-6.05 (m, H, CH═CH₂).

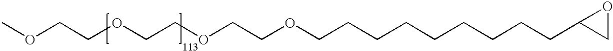

α-methoxy-ω-(O-11-undecane-1,2-dioxo)-poly(ethylene oxide 5k)

Prior to use, commercially available 3-Chloroperoxybenzoic acid (0.60 g, 60%, 2.1 mmol) was dissolved in toluene (100 mL) and dried over anhydrous sodium sulfate for two hours. The polymer from the previous step (5 g, 0.97 mmol) was added to 3-Chloroperoxybenzoic acid (80 mL of toluene solution) and stirred for two days. The solvent was removed in vacuo producing a white solid. The crude product was dissolved in CH₂Cl₂ and precipitated into anhydrous ethyl ether cooled in a dry ice-acetone bath. The precipitate was rinsed with ether twice and collected at the bottom of centrifuge tube (centrifugation at 10,000 rps for 30 min. at −20° C. The final product was dried in vacuum oven over P₂O₅ overnight and obtained as a soft yellow solid (4.25 g, 0.82 mmol, 85%). ¹H NMR (CDCl₃): δ 1.30-1.35 (b, 12H, (CH₂)₆), 1.55-1.65 (b, 2H, CH₂), 2.00-2.20 (b, 4H, 2×(CH₂)), 3.50-4.00 [b, 460H, (CH₂CH₂O)₁₁₃+OCH₂+CH—CH₂+OCH₃).

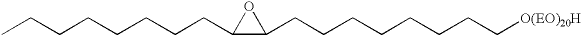

Polyoxyethylene(20)-9,10-dioxa-1-octadecylether (Brij98-epoxide).

Prior to use, commercially available 3-Chloroperoxybenzoic acid (11.7 g, 57%, 38.7 mmol) was dissolved in toluene (300 mL) and dried over anhydrous sodium sulfate overnight. Brij98 (25 g, 21.7 mmol) was added to 3-Chloroperoxybenzoic acid (280 mL of toluene solution) and stirred overnight. The solvent was removed in vacuo producing a yellow oil. The crude product was dissolved in CH₂Cl₂ and precipitated into anhydrous ethyl ether cooled in a dry ice-acetone bath. The precipitate was rinsed with ether twice and collected at the bottom of centrifuge tube (centrifugation at 10,000 rps for 30 min. at −20° C. The final product (27.8 g) was dried by vacuum oven over P₂O₅ overnight. ¹H NMR (CDCl₃): δ 0.98 (t, 3H, CH₃), 1.30-1.55 (b, 22H, (CH₂)₅ & (CH₂)₆), 1.56-1.75 (m, 6H, 3×(CH₂)), 3.60-3.90 (b, 84H, (CH₂CH₂O)₂₀ & CH₂ & OCHCHO). TLC (silica, R_{f}=0.66, CHCl₃/MeOH, 10:2, v/v).

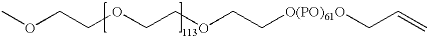

α-methoxy-ω-allyloxy-poly(oxyethylene5k)-poly(oxypropylene(61)) Co-Polymer; MeO(EO)₁₁₃(PO)₆₁O-allyl Ether

Potassium t-Butoxide (0.561 g, 5 mmol) was added to Poly(ethylene glycol) monomethyl ether 5k (25 g, 5 mmol) that was azeotropically dried using toluene 250 mL). The solvents were removed in vacuo resulting in a yellow viscous residue. he residue was dissolved in freshly distilled THF (200 mL) and immediately followed by addition of propylene oxide (38.35 g, 46.2 mL, 0.66 mol). The resulting solution was stirred and gently heated under Argon for two days. Sodium hydride (1.2 g, 5 equivalents, 25 mmol) was added and the mixture stirred at room temperature overnight. Allyl bromide (5 equivalents, 3.02 g, 25 mmol) was then added and the mixture stirred for 2 more days. The reaction was quenched with glacial acetic acid, inorganic salts removed by filtration, and solvent evaporated in vacuo. The crude product was obtained as an orange oil (64.21 g) and was used without further purification. The amount of propylene oxide incorporated to this co-polymer was determined by ¹H NMR as 61.

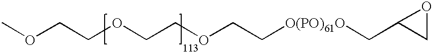

α-methoxy-ω-(3-oxy-1,2-dioxapropyl)-poly(oxyethylene5k)-poly(oxypropylene(61)) Co-Polymer; 1,2-epoxypropyl-3-ether-O(PO)₆₁(EO)₁₁₃OCH₃

3-Chloroperbenzoic Acid (2.9 g, 10.08 mmol) was added to Toluene (80 mL, 0.75 mmol) and dried over anhydrous Na₂SO₄ overnight. Solution became clear and yellow once it was dry. This solution was added to 20 g of allyl ether-O(PO)₆₁(EO)₁₁₃OCH₃; MeO(EO)₁₁₃(PO)₆₁OCH₂CHCH₂ (1373-079) and stirred over weekend. Solvent was then reduced in vacuo to 40 mL and product was precipitated from 600 mL ether that was chilled to −70° C. The solution becomes opalescent and was centrifuged at 10000 RPM at −20° C. to form a solid pellet. The solid was collected by centrifugation and rinsed three times with ethyl ether before drying under vacuum.

¹H NMR (D₂O): δ 1.1 (b, (—OCH₂CH(CH₃)—O)₆₁), 3.5-3.9 (b, (OCH₂CH₂—O)₁₁₃.

α-(p-isooctylphenyl)-ω-(O-11-bromoundecan)-poly(ethylene oxide) (Triton X-405-O—C10-Br,

The water was removed azeotropically from Triton X-405 (70% aqueous solution) (33.2 ml) with toluene (200 ml). Potassium t-butoxide was added (1.1 g, 0.01 ml). The solution was stirred at 25° C. under argon for 2 hr. Any remaining toluene was removed in vacuo. The remaining residue was re-dissolved in anhydrous THF (150 ml), 1,10-dibromodecane was added (6.0 g, 0.02 mol), and the yellow-orange solution was stirred at 25° C. overnight. Glacial acetic acid (3 ml) was then added to neutralize excess base. The solids were removed by filtration. The filtrate was concentrated in vacuo, and then precipitated into large excess of ethyl ether. The product was filtered and dried in vacuo. Yield 15 g (62%). Triton X-405-C₁₀—Br 1H NMR (CDCl₃) δ 7.25 (m, Ar—H); 6.817 (br-s, Ar—H); 3.64 (br-s, O—CH₂—CH₂O); 2.16 (m); 1.67 (s); 1.32 (s,); 0.69 (s, CH₃)

Poly-L-lysine-graft-R₁-graft-R₂-graft-R₃ Co-Polymers (R₁=PEG-Hydrophobe Derivative; R₂=None, R₃=None)

Poly-L-lysine-graft-(PEG-Hydrophobe) polymers were prepared by reacting a PEG-hydrophobe-electrophile with ε-NH₂ lysine groups under basic conditions. For individual co-polymers, the ratios of PEG-hydrophobe-electrophile to poly-L-lysine, PEG-hydrophobe-electrophile size, and poly-L-lysine size were varied. The conditions of the syntheses are summarized in Table 8 and the general procedure is described for Triton X-405-C10-Br and PEG-C₁₀—Br below.

Poly-L-Lysine 10k-graft-(ε-NH—C10-PEG2k)₉ (PL-A)

Lithium hydroxide (18.1 mg, 0.43 mmol) dissolved in water (0.5 ml) was added to a solution of MeOPEG2k-C₁₀—Br (1.4 g, 0.63 mmol) and Poly-L-Lysine 10k (150 mg, 0.016 mmol) in methanol (8 ml). The flask was sealed and incubated at 65° C. overnight. After 18 h, additional PEG2k-C₁₀—Br (160 mg. 0.072 mmol) and lithium hydroxide (2.6 mg, 0.062 mmol) were added, and the flask was sealed and incubated at 65° C. After 48 h incubation at 65° C., the solvent was removed in vacuo, the residue was redissolved in water, and pH was adjusted to 3.7 with glacial acetic acid. The product was purified by CM and G-25 column chromatography as described to yield 350 mg (76%). ¹H NMR (D₂O): δ 4.23 (s, Cα-H), 3.61 (m, (CH₂CH₂O)₄₅), 3.29 (s, OCH₃), 2.88 (m, CH₂), 1.59 (m, Lys-(CH₂) and (CH₂)₁₀), 1.23 (m, CH₂ and (CH₂)₁₀).

PL10k-graft-(ε-NH—C10-Triton X-405)

Lithium hydroxide (12.4 mg, 0.28 mmol) dissolved in water (1 ml) was added to a solution of PL10k (100 mg, 0.01 mmol) and Triton X-405-C10—Br (2.04 g, 0.44 mmol) in methanol (8 mL). The flask was sealed and incubated at 65° C. for 48 h. Then additional lithium hydroxide (2.6 mg) and Triton X-405-C10-Br (255 mg) were added, and the reaction mixture incubated at 65° C. for 48 h. The solvents were evaporated in vacuo and the residue was re-dissolved in 0.05 M Acetic Acid.

Purification

The solid product was first dissolved into 150 mL of a solution of 0.1 M of sodium phosphate buffer pH 6 containing 10% of methanol v/v and then loaded on SP Sepharose FF Cation-Exchange Column. After 10 column volume washes to remove excess unreacted PEG-hydrophobe starting material, the final product was eluted with 0.1 M NaOH containing 10% of methanol v/v. The ninhydrin positive fractions were combined and pH was adjusted to pH 4-5 by dropwise addition of acetic acid. The solvent was removed in vacuo and the residue re-dissolved in 0.05 M Acetic Acid in 30% methanol v/v. The product was purified by Sephadex G-25 column eluted with 0.05 M HAc in 30% methanol v/v. The ninhydrin positive fractions were combined and lyophilized to give the product as a white solid. The purity of the final conjugate was established by two analytical methods. Gel electrophoresis was performed to exclude contamination by poly-L-lysine and TLC to exclude free PEG-hydrophobe contamination. Typically, the final product did not contain unreacted poly-L-lysine or PEG-hydrophobe starting material.

TABLE 8
Poly-L-lysine-graft-PEG-Coupled-Hydrophobe Conjugates for Gene Delivery.

| PL-A | PEG2k-C₁₀—Br | 40 | 9 | 9.4 | 45 | 145.2 (+/−1.35) | 188.2 (+/−1.35) |
|---|---|---|---|---|---|---|---|
| PL-B | PEG2k-C₁₀—Br | 60 | 29 | 9.4 | 45 | 366.5 (+/−1.20) | 162.3 (+/−1.2) |
| | | | | | | 285.9 (+/−1.50) | 156.5 (+/−1.5) |
| PL-C | PEG2k-C₁₀—Br | 40 | 19.2 | 9.4 | 45 | NA | NA |
| PL-D | PEG2k-C₁₀—Br | 40 | 14.5 | 9.4 | 45 | 178.1 (+/−1.35) | NA |
| PL-E | PEG2k-C₁₀—Br | 17.5 | 4.4 | 9.4 | 45 | NA | NA |
| PL-F | PEG2k-C₁₀—Br | 80 | 36.5 | 9.4 | 45 | NA | NA |
| PL-G | PEG2k-C₁₀—Br | 35 | 12 | 9.4 | 45 | NA | 164.9 (+/−1.35)* |
| PL-H | PEG2k-C₁₀—Br | 40 | 5.5 | K48Cys | 48 | NA | NA |
| PL-I | TritonX-405-C₁₀— | 40 | 9 | 9.4 | 45 | 208.2 (+/−1.2) | 127.7 (+/−1.2) |
| | Br | | | | | 219.8 (+/−1.1) | 134.7 (+/−1.1) |
| PL-J | PEG5k-C₁₂—Br | 40 | 4.7 | 9.4 | 45 | 185.9 (+/−1.2) | 106.4 (+/−1.2) |
| | | | | | | 175.9 (+/−1.1) | 115.0 (+/−1.1) |
| PL-K | Igepal-C₁₀—Br | 40 | 3.2 | 9.4 | 45 | 70.8 (+/−1.2) | 1206.3 (+/−1.2) |
| PL-L | PEG0.75k-C₁₀—Br | 36 | 8.4 | 9.4 | 45 | NA | NA |
| PL-M | C₁₈-PEG4.4k-Br | 15 | 2.8 | 9.4 | 45 | NA | NA |
| PL-N | C₁₈-PEG5k-C₁₀— | 18 | 6.6 | 9.4 | 45 | NA | NA |
| | Br | | | | | | |
| PL-O | N—(C₁₀-PEG2k)—N—(C₁₂)—N— | 18 | 4 | 9.4 | 45 | NA | NA |
| | (COCH₂I) | | | | | | |
| PL-P | PEG2k-C₁₀—Br | 50 | 16 | 26 | 123 | NA | NA |
| PL-Q | PEG2k-C₁₀—Br | 200 | 38 | 26 | 123 | NA | NA |

*with OGP

### Example 3

### Random Grafts of PEG Coupled Hydrophobic Molecules on Cationic Chains

Materials

Poly-L-aspartic acid (P(Asp)) sodium salt 10K [DP (Vis) 76, Mw 10,400 (Vis); DP (LALLS) 57, Mw (LALLS) 7,800] and ethidium bromide were purchased from Sigma Chemical Co., St. Louis, Mo. Plasmid DNA (pCMVb, Clontech, Palo Alto, Calif. and pCMV-Luciferase was prepared by BIO 101 (San Diego, Calif.). Plasmid DNA preparation contained more than 90% covalently closed circular DNA as determined by agarose gel electrophoresis. Tetrahydrofuman (THF) was purchased from VWR and doubly distilled from sodium benzophenyl ketal. Polyethylene glycol (PEG) amino 5k (MW 5254; Substitution: 98% (¹HNMR), 98.2% (titration)) purchased from Shearwater Polymers, Inc. (Huntsville, Ala.) was dried in vacuo at 40° C. All other reagents were used without further purification. L-cysteine and 1-bromooctadecane were purchased from the Aldrich Chemical Co. Bis(trichloromethyl)carbonate (triphosgene) and Nε-Z-L-Lysine were purchased from Fluka Chemika. Potassium Hydroxide was obtained from VWR Scientific.

Instrumentation

¹H NMR spectra of the monomers and polymers were obtained on a 300 MHz ARX-300 Bruker spectrometer. IR Spectra were recorded on a Perkin-Elmer 1600 series FTIR as a KBr pellet or on NaCl plates.

Synthesis of L-Octadecylcysteine (CysC₁₈)

Potassium hydroxide (1.68 g, 0.03 mol) in absolute ethanol (50 ml), and L-cysteine (1.8 g, 0.015 mol) ground to fine powder, were added and stirred under argon for 5 min. 1-bromooctadecane was then added (5.0 g, 0.015 mol) and the reaction was stirred under argon at 50° C. After 2 hr, acetic acid (25 ml) was added, and the reaction mixture was filtered over a medium porosity fritted glass filter, and washed with absolute ethanol (2×25 ml). The resulting white solid was dried in vacuo; yield 4.6 g (82%). The product was characterized by ¹H NMR and IR and used without further purification.

N-Carboxyanhydride of L-Cys-S—C₁₈

Synthesis of N-Carboxyanhydride of L-Octadecylcysteine was carried out by the Fuchs-Farthing method using triphosgene. L-octadecylcysteine (6.0 g, 0.014 mol) was suspended in dry THF (30 ml). Bis(trichloromethyl) carbonate (2.2 g, 0.007 mol) was added as a solution in dry THF (10 ml). The solution was stirred at 50° C. for 2 hours. The reaction mixture was filtered over fritted glass filter (M porosity), and the filtrate was poured into hexanes (300 ml) and stored at −20° C. overnight. The precipitate was filtered, washed with cold hexane (3×50 ml) and dried in vacuo. The white solid was recrystallized from THF/hexanes three times until the melting point remained constant. (m.p. 83-86° C.). The product was characterized by ¹HNMR (CDCl₃) and IR.

N-Carboxyanhydrides of e-(Benzyloxycarbonyl)-L-lysine and of L-phenylalanine were Synthesized and Characterized as Previously Reported.

Synthesis of PEG-5k-block-(Cys-S—C₁₈)₁₀.

To a solution of L-octadecylcysteine-NCA (3.3 g, 0.007 mol) in anhydrous THF (20 ml) was added PEG5k-amine (3.6 g, 0.0007 mol) dissolved in anhydrous THF (55 ml). The solution was stirred under argon at 40° C. for 24 hours. The reaction was monitored by IR. After 24 hours, the reaction was divided into 3 equal portions. The first part was used to characterize the intermediate co-polymer. The other two parts were used for synthesis of the triblock co-polymers that are described below.

PEG5k-block-(CysC₁₈)₁₀-block-(Lys-Z)₁₂₀.

NCA-LysZ (9.2 g, 0.03 mol) was suspended in 100 mL THF and PEG5k-block-(CysC₁₈)₁₀—NH₂ (33 ml, 0.0002 mol) was added. The solution was stirred at 40° C. for 72 hours, and became very viscous after 24 hours. The solution was monitored by IR. After 72 hours, the solution was evaporated to dryness, and re-dissolved in 90 ml CHCl₃, and precipitated into ether (800 ml) to obtain 9.5 grams. The product was characterized by ¹H NMR (CDCl₃ and DMSO).

PEG5k-block-(CysC₁₈)₁₀-block-(LysZ)₄₅ was prepared as described above. The removal of ε-N-carboxybenzyl protecting group was performed as previously described. The resulting tri-block polymers PEG5k-block-(CysC₁₈)₁₀-block-(Lys)ₙ (where n=45, 120) were characterized by ¹H NMR (D₂O and DMSO), and by gel electrophoresis.

Polyplex Preparation

Polyplexes were prepared by rapidly adding an equal volume of plasmid DNA to a volume of the copolymer. DNA (2×) was prepared in water and copolymers were dissolved in the 2× diluent before mixing. Polyplex concentrations are reported by DNA content and were 10 μg/ml unless otherwise noted.

Exchange Reaction

Polyplexes were formulated at room temperature by rapidly mixing 500 μL of DNA (2×) and 500 μL of copolymer stock solution. Final DNA concentration was 50 μg/mL at a charge ratio of 1.0 (+/−) in 150 mM NaCl. Each polyplex solution was divided into five 200 μL aliquots and incubated at room temperature for 30 minutes. Anionic molecules were added to the polyplex aliquots in increasing amounts (charge ratio 1, 4, 7, 10, and 100 per phosphate group). The samples were then incubated for 20 hours and analyzed on agarose gel (0.6%).

Estimation of Polyplex Size

Light scattering measurements were determined on a Brookhaven Instruments Corporation 90 Plus particle size analyzer equipped with a 50 mW laser which emits light at a wavelength of 532 nm. Reagents were passed through a Nalgene 200 nm surfactant-free cellulose acetate filter prior to polyplex formation. Results are reported as effective diameter defined as the average diameter which is weighted by the intensity of light scattered by each particle and shown in Table 1.

### Example 4

### Animal Studies

Polyplexes were administered to 10 week-old female Balb/c mice (Charles River Laboratories, Wilmington, Mass.) by tail vein injection. Animals were anesthetized with a 80 μl intramuscular injection of a cocktail prepared from 20 ml isotonic saline, 7.5 ml ketamine (100 mg/ml), 3.8 ml xylazine (20 mg/ml) and 0.75 ml acepromazine (10 mg/ml) prior to treatment. Typically, 500 μl to as low as 200 μl of isotonic saline containing 15-20 μg of pDNA formulated with conjugate and any formulant was injected. For luciferase expression studies, animals were sacrificed 24 hours post-injection by asphyxiation with CO₂. Organs were excised and rinsed twice with phosphate buffered saline (PBS). Organ weight was determined gravimetrically and recorded. Organs were dounce homogenized in ten volumes of cell lysis buffer (100 mmol/L potassium phosphate pH 7.8, 0.2% Triton X-100). The resultant cell lysate was centrifuged for 5 min at maximum speed in a clinical centrifuge tube. The clear aqueous phase was collected from between the lipid layer on top and the cell pellet on the bottom of the tube. This clear lysate was further clarified by an additional 5 minute centrifugation at high speed in a microcentrifuge. The luciferase assay was performed on 0.1-100 μl of the final supernatant. The luciferase activity of aliquots of tissue homogenate was measured with an Analytical Luminescence 2010 Luminometer. Background measurement was subtracted and the relative light units were converted to picograms of protein as calculated from standard curves based on purified luciferase protein standards (Analytical Luminescence Laboratories, San Diego, Calif.). In some studies, polyplexes were administered to anesthetized Buffalo, SHR, or Lewis rats by tail vein injection of 5 ml of the polyplex.

Serum samples for interferon measurements were obtained at various time points by retro-orbital puncture and stored at −70° C. prior to assay. Serum concentrations of IFN-α2b were measured using an ELISA kit (Endogen Inc., Cambridge, Mass.) according to the manufacturer's protocol. The ELISA is specific for human IFN-α2b and does not cross react with murine IFN. Non-specific signal was accounted for by subtracting 3× background level from each value. Animal data are reported as mean with standard deviation.

Fluorescent Studies.

For fluorescent localization studies, 0.5 ml of fluorescent CY5 polyplex was injected into the tail vein of 12 week-old Balb/C mice. Five minutes after injection, the animals were killed by cervical dislocation and the livers excised and rinsed in PBS. Liver tissue was cut into 2 mm by 2 mm squares and fixed in 4% paraformaldehyde for 4 hours. Tissue was infused in 0.5 molar sucrose overnight and then frozen in liquid nitrogen chilled isopentane. Frozen tissue was cut on a Leica cryostat at 10 mm and allowed to air dry for tissue attachment to slides. Liver sections were counterstained with the nuclear stain DAPI, mounted with immunomount (Shandon Lipshaw, Pittsburgh, Pa.), and viewed on an Olympus BH2 microscope equipped with filter cubes designed for emission wavelengths of 461 nm (DAPI) and 670 nm (Cy5). Images were captured and superimposed on one another using a CCD camera and Metamorph software.

Ultrasound Application

Animals were anesthetized (see above) approximately 15 minutes prior to treatment. The abdomens were shaved with Oster Finisher clippers and ultrasound gel was applied. Ultrasound was administered twice for 30 seconds at 0.5 minutes prior to and 5 minute after polyplex administration. Ultrasound was administered (1 MHz, 2W/cm2, 10% duty cycle) with a Therasound 2.5 instrument using a 2 cm 2 head (Rich-Mar, Inola, Okla.). The data for the in vivo experiments is summarized in Table 10.

The effect of different formulations of polyplexes on the expression on Luciferase per gram of liver was tested by injection mice with 0.5 mL of PLL-PEG and PLL-(C₁₀-PEG) polyplexes with formulants. The formulation contained 15 μg of pCMVLuc and measurements were taken after twenty four hours. The data shows that the addition of formulants such as Brij 35, OGP, TCDC and DHPC increase expression by about 100,000. Only low levels of Luciferase expression are detected when pCMVLuc is injected without formulant.

Furthermore, other formulants have also been tested. Table 9 shows the results of in vivo studies. The mice were injected with a 200 Cl dose, which contained 15 μg of DNA per injection. The structure of the steroidal fomulant is given below the table.

TABLE 9

| | | Luciferase | |
|---|---|---|---|
| | | Average | |
| Formulant | Dose | [pg./g liver] | SD |
| 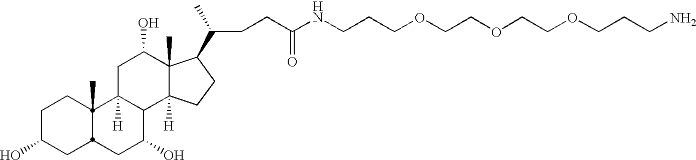 | 0.10% | 3964 | 8156 |
| TCDC | 0.50% | 517 | 564 |
| DHPC | 0.40% | 470 | 420 |
| CHAPSO | 0.50% | 188 | 217 |
| CHAPS | 0.50% | 178 | 236 |
| Deoxy BIGCHAP | 0.50% | 129 | 186 |
| CHENO CHAPS | 0.50% | 18 | 26 |
| TransIT | mfg | 0 | 0 |
| | spec. | | |

FIG. 13 shows the effects of varying polyplexes and using a formulant to enhance luciferase expression. The mice were injected with a 0.5 mL dose which contained 15 μg/mL of DNA (pCMVLuc). It was found that the polyplex which was constructed from the copolymer PLL9.4k-g-(ε-NH—C10-PEG2k)₁₄ when administered with DHPC, TCDC, OGP, Brij 35 resulted in enhanced expression of the gene luciferase. Furthermore, enhancement was also found, to a lesser degree, when the DNA was administered without a penetration enhancer in a polyplex of the invention.

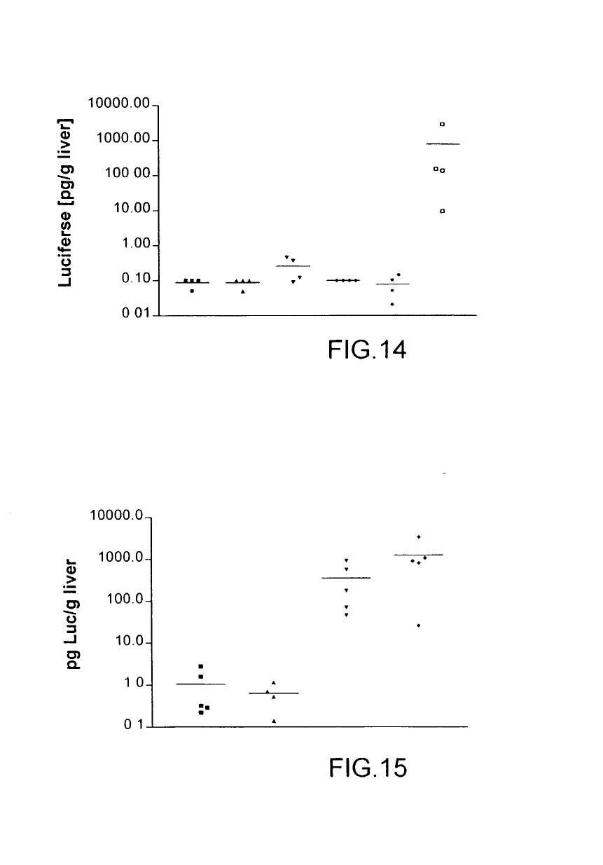

FIG. 14 shows the effects of varying polyplexes and using a formulant to enhance luciferase expression. The mice were injected with a 0.5 mL dose which contained 15 μg/mL of DNA (pCMVLuc). It was found that the polyplex which was constructed from the copolymer PLL9.4k-g-(ε-NH—C10-PEG2k)₁₄ when administered with DHPC resulted in enhanced expression of the gene luciferase, as compared to the other formulations tested in this trial.

FIG. 15 shows that the addition of the formulant, DHPC, greatly enhances the expression of luciferase. As above, the mice were injected a dose of 200 μL which contained 15 μg/mL of DNA (pCMVLuc). Both (PLL9.4k-g-(ε-NH—CO-“Trigal”)_{16.1}) and (PLL9.4k-g-(ε-NH—C12-PEG5K)_{4.7}-g-(ε-NH-“Trigal”) (LG-E) advantageously allowed for enhanced expression of luciferase in vivo with and without the addition of formulant.

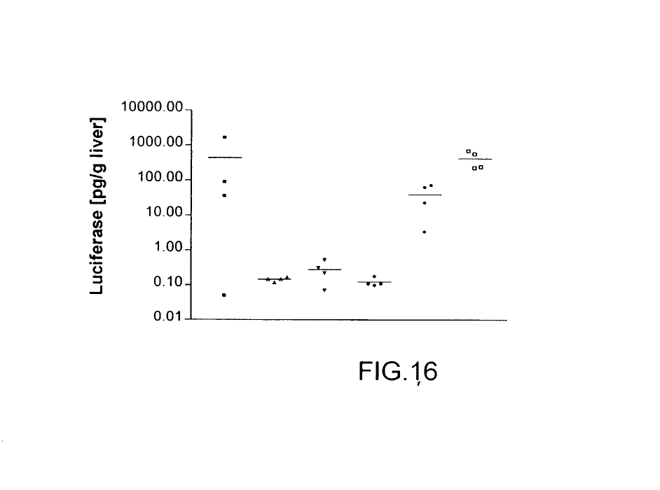

FIG. 16 shows that when mice were injected with a dose of 200 μl containing 15 μg/mL of DNA (pCMVLuc), expression of luciferase was dependent on the architecture of the conjugate used. It was found that the conjugate comprised of random grafts of PEG and the hydrophobe Cholesterol (10KPL-5 KPEG-cholesterol) had a wide range of luciferase expression when administered with the formulant, DHPC (represented by ‘●’). The range of luciferase expression ranged from below 0.1 pg Luc per gram of liver to over 1000 g of Luc per gram of liver. The symbol ′ represents Polyplexes comprised of block co-polymer (PEG5k-b-(Cys-S—C18)₁₀-b-(Lys)₄₅) and (PEG5k-b-(Phe)₁₄-b-(Lys)₅₁) (represented by ‘▴’ and ‘♦’, respectively), resulted in some luciferase expression. Polyplexes comprised of polymers consisting of random grafts of PEG-coupled-hydrophobe with and without Trigalactose ligand included PLL9.4k-g-(ε-NH-PEG4.4k-C18)_{2 8}(‘▾’), PLL10k-g-(ε-NH—C10-PEG4.4k-C18)_{6.6} (‘♦’), and PLL9.4k-g-(ε-NH—C₁₂-PEG5k)_{4.7}-g-(ε-NH—CH2CO-“Trigal”)₉(‘□’). When administered with DHPC, the resulting expression of DNA expression was enhanced.

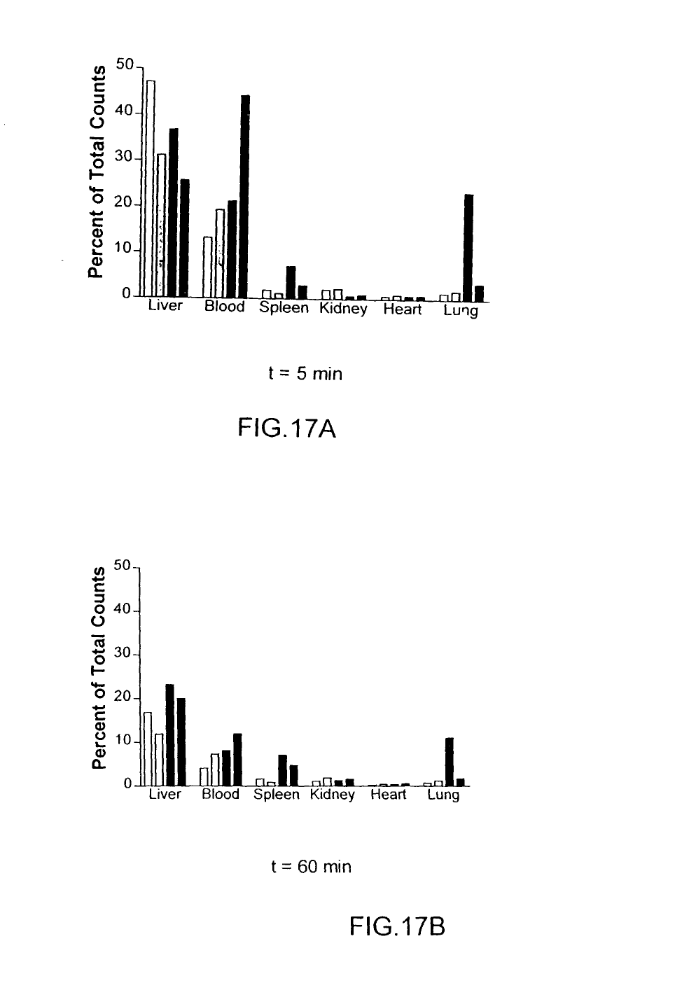

The biodistribution of ¹²⁵I-pCMV βGal was also studied in mice. Mice were injected with a dose of 300 μL containing 50 μg/mL of DNA. The distribution of free DNA, free DNA and TCDC, encapsulated in a polyplex comprised of block copolymer (PEG5k-b-(Cys-S—C18)₁₀-b-(Lys)₄₅) and the polyplex with TCDC were tested after 5 (FIG. 17A) and 60 (FIG. 17B) minutes. It was found that TCDC redirects the polyplex from the lungs and other organs to the blood. The clearance of ¹²⁵I-pCMVβGal was also studied using the ABC Polymer, (PEG5k-b-(Cys-S—C18)₁₀-b-(Lys)₄₅) (BP-A). It was found that the administration of TCDC greatly enhanced the half-life of the DNA.

1. A method of delivering an anionic agent through a lipid membrane comprising:
a) contacting the anionic agent with a delivery enhancing formulation, comprising a cationic backbone moiety, a hydrophobic moiety, and a hydrophilic moiety;
b) allowing a polyplex to form; and
c) contacting the lipid membrane with a penetration enhancer, such that upon contact of the polyplex with the lipid membrane, the anionic agent is delivered through the membrane.

2. The method of claim 1, wherein said lipid membrane is a cellular membrane.

3. The method of claim 1, wherein said lipid membrane is a nuclear membrane.

4. The method of claim 1, wherein said lipid membrane is an endosomal membrane.

5. The method of claim 1, wherein said cationic backbone moiety is non-peptidic.

6. The method of claim 5, wherein said cationic backbone moiety is polyethylenimine.

7. The method of claim 1, wherein said cationic backbone is peptidic.

8. The method of claim 7, wherein said cationic backbone is polylysine.

9. The method of claim 8, wherein said polylysine backbone as a molecular weight from about 5 to about 50 K.

10. The method of claim 1, wherein said hydrophobic moiety is comprised of moieties selected from the group consisting of substituted and unsubstituted alkyl, substituted and unsubstituted alkenyl, substituted and unsubstituted alkynyl, substituted and unsubstituted aryl, peptides and combinations thereof.

11. The method of claim 10, wherein said hydrophobic moiety contains from 4 to 40 carbon atoms

12. The method of claim 1, wherein said hydrophobic moiety comprises a steroidal nucleus.

13. The method of claim 12, wherein said steroidal nucleus is cholesterol.

14. The method of claim 1, wherein said hydrophobic moiety modifies about 0.5% to about 85% of cationic charges on said cationic backbone.

15. The method of claim 1, wherein said hydrophilic moiety is poly(oxyalkylene) glycol.

16. The method of claim 15, wherein said hydrophilic moiety is poly(oxyethylene glycol).

17. The method of claim 16, wherein the molar ratio of said poly(oxyethylene glycol) chains to the cationic backbone is from about 1 to about 40.

18. The method of claim 1, wherein said hydrophilic moiety is poly(ethyloxazoline) or poly(methyloxazoline).

19. The method of claim 18, wherein the molar ratio of the poly(ethyloxazoline) or poly(methyloxazoline) chains to the cationic backbone is from about 1 to about 40.

20. The method of claim 1, wherein said penetration enhancer is selected from the group consisting of non-ionic agents, negatively charged ionic agents, cationic agents, zwitterionic agents, lipid derivatives, fluorinated agents, natural products, synthetic products, and mixtures thereof.

21. The method of claim 20, wherein said non-ionic agent is selected from the group consisting of Brij surfactants, oleyl surfactants; Igepal CO-990, Tween 20, Tween 40, Tween 60, Tween 80, Triton X-405, Triton X-100, Tetronic 908, Cholesterol PEG 900, Polyoxyethylene Ether W-1; Span 20, Span 40, Span 85, azones and mixtures thereof.

22. The method of claim 21, wherein said Brij surfactant is selected from the group consisting of Brij 30, Brij 35, Brij 36, Brij 52, Brij 56, Brij 58, Brij 72, Brij 76, Brij 78, Brij 92, Brij 96, Brij 97, Brij 98, Brij 98/99, and combinations thereof.

23. The method of claim 21, wherein said oleyl surfactant is selected from the group consisting of oleyl-EO₀, oleyl-EO₂, oleyl-EO₅, and oleyl-EO₁₀.

24. The method of claim 21, wherein said azone is selected from the group consisting of N-ethyl-aza-cycloheptanones, N-hexyl-aza-cycloheptanones, N-octyl-aza-cycloheptanones, N-decyl-aza-cycloheptanones, N-dodecyl-aza-cycloheptanones, N-tetradecyl-aza-cycloheptanones, and N-hexadecyl-aza-cycloheptanones.

25. The method of claim 20, wherein said non-ionic agent is selected from the group consisting of n-hexyl-β-glucopyranoside, n-heptyl-β-glucopyranoside, α-octyl-β-gluopyranoside, n-dodecyl-β-glucopyranoside, n-octyl-α-glucopyranoside, phenyl-β-glucopyranoside, n-hexyl-β-(D-1-thioglucopyranosides), n-heptyl-β-(D-1-thioglucopyranosides), n-octyl-β-(D-1-thioglucopyranosides), n-dodecyl-β-(D-1-thioglucopyranosides), n-octyl-β-(D)-galactopyranosides, n-dodecyl-β-(D)-galactopyranosides, N-decanoyl-N-methyl-glucamine, N-octanoyl-N-methyl-glucamine, and mixtures thereof.

26. The method of claim 20, where said negatively charged ionic agent is selected from the group consisting of: N-lauryl sarcosine salt, linolic acid salt, cholesteryl hydrogen succinate, DSPE-PEG, bile acid, hydrotropes, and mixtures thereof.

27. The method of claim 26, wherein said hydrotrope is 8-(5-carboxy-4-hexyl-cyclohex-2-enyl)-octanoic acid.

28. The method of claim 26, wherein said bile acid is selected from the group consisting of natural and synthetic bile acids, conjugated bile acids, mixtures, and pharmaceutically acceptable salts thereof.

29. The method of claim 28, wherein said bile acid is selected from the group consisting of lithocholate, deoxycholate, glycodeoxycholate, taurodeoxycholate, chenodeoxycholate, glycochenodeoxycholate, taurochenodeoxycholate, ursodeoxycholate, glycoursodeoxycholate, tauroursodeoxycholate, cholate, glycocholate, taurocholate, ursocholate, glycoursocholate, tauroursocholate, pharmaceutically acceptable salts and combinations thereof.

30. The method of claim 29 wherein said bile acid is taurochenodeoxycholate.

31. The method of claim 20, wherein said cationic or said zwitterionic agent is selected from the group consisting of 2-undecylimidazole, 2-heptadecylimidazole, N,N-dimethylnonylamine-N-oxide, N,N-dimethyloctadecylamine-N-oxide, and mixtures thereof.

32. The method of claim 20, wherein said lipid derivative is selected from the group consisting of 1,2-diheptanoyl-sn-glycero-3-phosphocholine, 1,2-dioctanoyl-sn-glycero-3-phosphocholine, and mixtures thereof.

33. The method of claim 20, wherein said per-fluorinated agent is selected from the group consisting of Zonyl FSN 100, Zonyl FSA and mixtures thereof.

34. The method of claim 20, wherein said natural or synthetic product is selected from the group consisting of nystatin, natural and synthetic saponins, β-carotene, and chloroquine diphosphate.

35. The method of claim 1, wherein said anionic agent is polymeric.

36. The method of claim 35, wherein said anionic agent is a nucleic acid.

37. The method of claim 36, wherein said nucleic acid is DNA.

38. The method of claim 37, wherein said nucleic acid comprises a DNA sequence which encodes a genetic marker selected from the group consisting of luciferase, α-galactosidase, hygromycin resistance, neomycin resistance, green fluorescent protein and chloramphenicol acetyl transferase.

39. The method of claim 37, wherein the nucleic acid comprises a DNA sequence encoding a protein selected from the group consisting of low density lipoprotein receptors, coagulation factors, suppressors of tumors, cytokines, angiogenesis factors, tumor antigens, immune modulators, major histocompatibility proteins, antioncogenes, p16, p53, thymidine kinase, IL2, IL4, IL10, and TNFα.

40. The method of claim 37, wherein said nucleic acid encodes for a viral protein, a bacterial protein, or a cell surface marker.

41. The method of claim 36, wherein said nucleic acid encodes an RNA selected from the group consisting of a sense RNA, an antisense RNA, and a ribozyme.

42. The method of claim 36, wherein said nucleic acid encodes a protein selected from the group consisting of lectin, a mannose receptor, a sialoadhesin, and a retroviral transactiviating factor.

43. The method of claim 1, wherein said polyplex further comprises one or more cellular ligands, nuclear ligands, or an endosomal escape mechanisms.

44. The method of claim 1, wherein said delivery enhancing formulation is selected from the group consisting of Br(CH₂)₁₀CO—NH-β-lactosyl amide, N¹-(bromoacetamide)-N¹³-(chenodeoxycholic acid amide)-4,7,10-trioxo-1,13-tridecanediamine, 1,1,1-tris-[(O¹⁶-β-D-galactopyranoside)-7,10,13,16-tetraoxo-5-one-4-aza-hexadecanyl]-1-[1-aza-11-amino-2-one-undecanyl]-methane; 1,1,1-tris-[(O¹⁶-β-D-galactopyranoside)-7,10,13,16-tetraoxo-5-one-4-aza-hexadecanyl]-1-[1,11-diaza-2,12-dione-13-bromotridecanyl]-methane; N¹-(iodoacetamide)-N¹³-(cholic acid amide)-4,7,10-trioxo-1,13-tridecanediamine; and BrCH₂CONH(CH₂)₃—O-β-cholesterol ether.

45. A method of enhancing expression of a nucleic acid in a cell, comprising:
a) contacting the nucleic acid with a delivery enhancing formulation comprising a cationic backbone moiety, a hydrophobic moiety, and a hydrophilic moiety;
b) allowing a polyplex to form;
c) contacting the membrane of the cell with a penetration enhancer, such that upon contact of the polyplex with the membrane of the cell, the nucleic acid is internalized into the cell and expression of said nucleic acid is enhanced.

46. The method of claim 44, wherein said penetration enhancer is a cholanic or a chenodeoxycholanic acid derivative.

47. The method of claim 45, wherein said penetration enhancer is N′-(Cholic Acid Amide)-4,7,10-trioxo-1,13-tridecanediamine; N¹-(Chenodeoxycholic Acid Amide)-4,7,10-trioxo-1,13-tridecanediamine; or N-Chenodeoxycholyl-2-aminoethyl-phosphonic acid monopotassium salt.

48. The method of claim 44, wherein said cationic backbone moiety is polylysine.

49. The method of claim 47, wherein said polylysine backbone is 5K to 50 K.

50. The method of claim 44, wherein said hydrophobic moiety is poly(oxyalkylene) glycol.

51. The method of claim 49, wherein said hydrophilic moiety is poly(oxyethylene glycol).

52. The method of claim 49, wherein the molar ratio of said poly(oxyalkylene) glycol chains to the cationic backbone is from about 1 to about 40.

53. The method of claim 44, wherein said nucleic acid is DNA.

54. The method of claim 52, wherein said nucleic acid comprises a DNA sequence which encodes a genetic marker selected from the group consisting of luciferase, β-galactosidase, hygromycin resistance, neomycin resistance, green fluorescent protein and chloramphenicol acetyl transferase.

55. The method of claim 53, wherein the nucleic acid comprises a DNA sequence encoding a protein selected from the group consisting of low density lipoprotein receptors, recombinant proteins, coagulation factors, suppressors of tumors, cytokines, angiogenesis factors, tumor antigens, immune modulators, anti-inflammatory proteins, major histocompatibility proteins, enzymes, antioncogenes, p16, p53, thymidine kinase, interleukins, IL2, IL4, IL10, and TNFα.

56. The method of claim 52, wherein said nucleic acid encodes for a protein selected from the group consisting of a viral antigens, a bacterial protein, and cell surface markers.

57. The method of claim 56, wherein said viral antigen is selected from the group consisting of HIV, HIV p24, HSV gD, and HBV S.

58. A method for treating a subject comprising administering to said subject an effective amount of a penetration enhancer and a polyplex comprising a nucleic acid, a cationic backbone moiety, a hydrophobic moiety, and a hydrophilic moiety, such that said subject is treated.

59. The method of claim 57, wherein said subject is a human.

60. The method of claim 58, wherein said human is suffering from a genetic disorder.

61. The method of claim 58, wherein said human is suffering from an acquired disease.

62. The method of claim 57, wherein said anionic agent is a nucleic acid.

63. The method of claim 57, wherein said polyplex comprises a poly lysine back bone moiety and a hydrophobic moiety, and a poly(oxyethylene glycol) hydrophilic moiety.

64. The method of claim 57, wherein said penetration enhancer is a cholanic acid or a chenodeoxycholanic acid derivative.

65. The method of claim 61, wherein said nucleic acid is associated with a genetic disorder or an acquired disease.

66. The method of claim 57, wherein said polyplex is administered by a method selected from the group consisting of systemic, regional, topical, perfusive, injection, intramuscular, intraperitoneal, subcutaneous, intradermal, and oral administration.

67. The method of claim 57, further comprising administering a pharmaceutically effective carrier.

68. The method of claim 57, wherein the subject is treated for a disorder selected from the group consisting of hepatitis, inflammatory diseases, hemophilia, metabolic deficiencies, metabolic disorders, immune rejection of transplanted tissue, infections by invading pathogens, tissue trauma, ischemia, lipid metabolism disorders, cholesterolimia, hypercholesterolimia, peripheral and central nervous system disorders and regeneration, obesity, allergies, allergic rhinitis, asthma, Gaucher's disease, epilepsy, Parkinson's disease, ocular diseases, elevated intraocular pressure, cancer, skin disorders, and alopecia.

69. The method of claim 57, wherein said polyplex is comprised of random grafts of hydrophobic moieties and random grafts of hydrophilic moieties on a cationic backbone moiety.

70. The method of claim 68, wherein said cationic backbone moiety is poly-L-lysine.

71. The method of claim 68, wherein said hydrophilic moiety is poly(oxyethylene glycol).

72. The method of claim 57, wherein said polyplex is comprised of a polymer of the formula:
A-B-C
wherein
A is a hydrophilic domain;
B is a hydrophobic domain;
C is a cationic domain.

73. The method of claim 57, wherein said polyplex is comprised of a polymer selected from the group consisting of poly-L-lysine-graft-(ε-NH-PEG5k)_{12.8}-graft-(ε-NH—CH₂CONHCH₂CH₂CH₂—O-β-cholesterol ether)₂₆; poly-L-lysine9.6k-graft-(ε-NH-PEG3k)_{12.6}-graft-(ε-NH—(CH₂)₁₀—CO—NH-Lactose)_{8.8}; or PLL9.4k-graft-(ε-NH—C10-PEG2k)_{4.7}-graft-(ε-NH—CH₂CONH(CH₂)₁₀—CO—NH-Trigalactose)₉.

74. The method of claim 57, wherein said polyplex is comprised of a polymer selected from the group consisting of PLL10k-graft-(ε-NH—C10-PEG2k)₉; PL10k-graft-(ε-NH—C10-Triton X-405)₉; PL9.4k-graft-(ε-NH—C10-Igepal-CO-990)_{3.2}; PLL9.4k-graft-(ε-NH-Brij700)_{2.8}; PLL9.4k-graft-(ε-NH—C10-Brij700)_{6.6}; PLL9.4k-graft-(ε-NH—CH₂CH(OH)(CH₂)₉-PEG5k)_{6.5}; PLL9.4k-graft-(ε-NH-Brij⁹⁸)₁₁; PLL9.4k-graft-(NH-Brij98)₆; PLL9.4k-graft-(ε-NH—CH₂CH(OH)CH₂O(PO)₆₁(EO)₁₁₃OCH₃)_{9.8}; PLL9.4k-graft-(ε-NH—CH₂CH(OH)CH₂O(PO)₆₁(EO)₁₁₃OCH₃)_{24.6}; polyethylenimine-graft-(—NH—CH₂CH(OH)CH₂O(PO)₆₁(EO)₁₁₃OCH₃)₇; or polyethylenimine-graft-(—NH—CH₂CH(OH)CH₂O(PO)₆₁(EO)₁₁₃OCH₃)₁₅.

75. The method of claim 57, wherein said polyplex is comprised of PEG5k-block-(CysC₁₈)₁₀-block-(Lys)₄₅; or PEG5k-block-(CysC₁₈)₁₀-block-(Lys)₁₂₀.

76. A polyplex comprising an anionic agent, and a polymer selected from the group consisting of poly-L-lysine-graft-(ε-NH-PEG5k)_{12.8}-graft-(ε-NH—CH₂CONHCH₂CH₂CH₂—O-β-cholesterol ether)₂₆; poly-L-lysine9.6k-graft-(ε-NH-PEG3k)_{12.6}-graft-(ε-NH—(CH₂)₁₀—CO—NH-Lactose)_{8.8}; and PLL9.4k-graft-(ε-NH—C10-PEG2k)_{4.7}-graft-(ε-NH—CH₂CONH(CH₂)₁₀-CO—NH-Trigalactose)₉.

77. A polyplex comprising an anionic agent and a polymer selected from the group consisting of PLL10 k-graft-(ε-NH—C 10-PEG2k)₉; PL10k-graft-(ε-NH—C10-Triton X-405)₉; PL9.4k-graft-(ε-NH—C10-Igepal-CO-990)_{3.2}; PLL9.4k-graft-(ε-NH-Brij700)_{2.8}; PLL9.4k-graft-(ε-NH—C10-Brij700)_{6.6}; PLL9.4k-graft-(ε-NH—CH₂CH(OH)(CH₂)₉-PEG5k)_{6.5}; PLL9.4k-graft-(ε-NH-Brij98)₁₁; PLL9.4k-graft-(NH-Brij98)₆; PLL9.4k-graft-(ε-NH—CH₂CH(OH)CH₂O(PO)₆₁(EO)₁₁₃OCH₃)_{9.8}; PLL9.4k-graft-(ε-NH—CH₂CH(OH)CH₂O(PO)₆₁(EO)₁₁₃OCH₃)_{24.6}; polyethylenimine-graft-(—NH—CH₂CH(OH)CH₂O(PO)₆₁(EO)₁₁₃OCH₃)₇; and polyethylenimine-graft-(—NH—CH₂CH(OH)CH₂O(PO)₆₁(EO)₁₁₃OCH₃)₁₅.

78. A polyplex comprising an anionic agent and a polymer selected from the group consisting of PEG5k-block-(CysC₁₈)₁₀-block-(Lys)₄₅; or PEG5k-block-(CysC₁₈)₁₀-block-(Lys)₁₂₀.

79. The polyplex of claim 75-77, wherein said anionic agent is a nucleic acid.

80. The polyplex of claim 78, wherein said nucleic acid is DNA.

81. The polyplex of claim 79, wherein said nucleic acid comprises a DNA sequence which encodes a genetic marker selected from the group consisting of luciferase gene, β-galactosidase gene, hygromycin resistance, neomycin resistance, green fluorescent protein and chloramphenicol acetyl transferase.

82. The polyplex of claim 79, wherein said nucleic acid comprises a DNA sequence encoding a protein selected from the group consisting of low density lipoprotein receptors, coagulation factors, suppressors of tumors, cytokines, angiogenesis factors, tumor antigens, immune modulators, major histocompatibility proteins, antioncogenes, p16, p53, thymidine kinase, IL2, IL4, IL 10, and TNFα.

83. The polyplex of claim 78, wherein said nucleic acid encodes for a viral protein, a bacterial protein, a cell surface marker, HIV antigens, HIV p24 antigens, HSV gD antigens, HBV S antigens.

84. The polyplex of claim 78, wherein said nucleic acid encodes an RNA selected from the group consisting of a sense RNA, an antisense RNA, and a ribozyme.

85. The polyplex of claim 78, wherein said nucleic acid encodes a lectin, a mannose receptor, a sialoadhesin, or a retroviral transactiviating factor.

86. The polyplex of claim 75-77, further comprising Br(CH₂)₁₀CO—NH-β-lactosyl amide, N¹-(bromoacetamide)-N¹³-(chenodeoxycholic acid amide)-4,7,10-trioxo-1,13-tridecanediamine; or N¹-(iodoacetamide)-N¹³-(cholic acid amide)-4,7,10-trioxo-1,13-tridecanediamine; BrCH₂CONH(CH₂)₃—O-β-cholesterol ether.

87. The polyplex of claim 75-77, further comprising a trigalactose-ligand-amine.

88. The polyplex of claim 86, wherein said trigalactose-ligand-amine is 1,1,1-Tris-[(O¹⁶-β-D-Galactopyranoside)-7,10,13,16-tetraoxo-5-one-4-aza-hexadecanyl]-1-[1-aza-11-amino-2-one-undecanyl]-methane.

89. The polyplex of claim 75-77 further comprising a trigalactose-ligand-bromoacetamide.

90. The polyplex of claim 88, wherein said trigalactose-ligand-bromoacetamide is 1,1,1-Tris-[(O¹⁶-β-D-Galactopyranoside)-7,10,13,16-tetraoxo-5-one-4-aza-hexadecanyl]-1-[1,11-diaza-2,12-dione-13-bromotridecanyl]-methane.

91. A pharmaceutical composition comprising an effective amount of, a penetration enhancer, a pharmaceutically acceptable carrier and a polyplex, wherein said polyplex is comprised of a cationic backbone moiety, a hydrophobic moiety, an anionic agent, and a hydrophilic moiety.

92. The pharmaceutical composition of claim 90, wherein said cationic backbone is polylysine.

93. The pharmaceutical composition of claim 91, wherein said poly lysine backbone has a molecular weight from about 5 to about 50 K.

94. The pharmaceutical composition of claim 90, wherein said hydrophilic moiety is poly(oxyethylene glycol).

95. The pharmaceutical composition of claim 90, wherein said hydrophilic moiety is poly(ethyloxazoline) or poly(methyloxazoline).

96. The pharmaceutical composition of claim 90, wherein said penetration enhancer is selected from the group selected from non-ionic agent, a negatively charged ionic agent, a cationic agent, a zwitterionic agent, a lipid derivative, a per-fluorinated agent, a natural product, synthetic products, and mixtures thereof.

97. The pharmaceutical composition of claim 95, where said negatively charged ionic agent is selected from the group consisting of N-lauryl sarcosine salt, linolic acid salt, cholesteryl hydrogen succinate, DSPE-PEG, bile acid, hydrotropes, and mixtures thereof.

98. The pharmaceutical composition of claim 96, wherein said bile acid is taurochenodeoxycholate.

99. The pharmaceutical composition of claim 90, wherein said anionic agent is a nucleic acid.

100. The pharmaceutical composition of claim 90, wherein said effective amount is effective to treat a genetic disorder.

101. The pharmaceutical composition of claim 98, wherein said effective amount is effective to treat a non-genetic disease.

102. The pharmaceutical composition of claim 90, wherein said pharmaceutically acceptable carrier is suitable for systemic, regional, topical, perfusive, injection, intramuscular, intraperitoneal, subcutaneous, intradermal, or oral administration.

103. The pharmaceutical composition of claim 90, wherein said polyplex is comprised of poly-L-lysine-graft-(ε-NH-PEG5k)_{12.8}-graft-(ε-NH—CH₂CONHCH₂CH₂CH₂—O-β-cholesterol ether)₂₆; poly-L-lysine9.6k-graft-(ε-NH-PEG3k)_{12 6}-graft-(ε-NH—(CH₂)₁₀—CO—NH-Lactose)_{8.8}; or PLL9.4k-graft-(ε-NH—C10-PEG2k)_{4.7}-graft-(ε-NH—CH₂CONH(CH₂)₁₀—CO—NH-Trigalactose)₉.

104. The pharmaceutical composition of claim 90, wherein said polyplex is comprised of a polymer selected from the group consisting of PLL10k-graft-(ε-NH—C10-PEG2k)₉; PL10k-graft-(ε-NH—C10-Triton X-405)₉; PL9.4k-graft-(ε-NH—C10-Igepal-CO-990)_{3.2}; PLL9.4k-graft-(ε-NH-Brij700)_{2.8}; PLL9.4k-graft-(εNH—C10-Brij700)_{6.6}; PLL9.4k-graft-(εNH—CH₂CH(OH)(CH₂)₉-PEG5k)_{6.5}; PLL9.4k-graft-(ε-NH-Brij98)₁₁(1401-080); PLL9.4k-graft-(NH-Brij98)₆; PLL9.4k-graft-(-ε-NH—CH₂CH(OH)CH₂O(PO)₆₁(EO)₁₁₃OCH₃)_{9.8}; PLL9.4k-graft-(ε-NH—CH₂CH(OH)CH₂O(PO)₆₁(EO)₁₁₃OCH₃)_{24.6}; polyethylenimine-graft-(—NH—CH₂CH(OH)CH₂O(PO)₆₁(EO)₁₁₃OCH₃)₇; or polyethylenimine-graft-(—NH—CH₂CH(OH)CH₂O(PO)₆₁(EO)₁₁₃OCH₃)₁₅.

105. The pharmaceutical composition of claim 90, wherein said polyplex is a comprised of PEG5k-block-(CysC₁₈)₁₀-block-(Lys)₄₅; or PEG5k-block-(CysC₁₈)₁₀-block-(Lys)₁₂₀.

106. A polymer of the formula: poly-L-lysine-graft-(ε-NH-PEG5k)_{12.8}-graft-(ε-NH—CH₂CONHCH₂CH₂CH₂—O-β-cholesterol ether)₂₆.

107. A polymer selected from the group consisting of: PLL10k-graft-(ε-NH—C10-PEG2k)₉; PL10k-graft-(ε-NH—C10-Triton X-405)₉; PL9.4k-graft-(ε-NH—C10-Igepal-CO-990)_{3.2}; PLL9.4k-graft-(ε-NH-Brij700)_{2.8}; PLL9.4k-graft-(ε-NH—C10-Brij700)_{6 6}; PLL9.4k-graft-(ε-NH—CH₂CH(OH)(CH₂)₉-PEG5k)_{6.5}; PLL9.4k-graft-(ε-NH-Brij98)₁₁; PLL9.4k-graft-(NH-Brij98)₆; PLL9.4k-graft-(-ε-NH—CH₂CH(OH)CH₂O(PO)₆₁(EO)₁₁₃OCH₃)_{9.8}; PLL9.4k-graft-(ε-NH—CH₂CH(OH)CH₂O(PO)₆₁(EO)₁₁₃OCH₃)_{24.6}; polyethylenimine-graft-(—NH—CH₂CH(OH)CH₂O(PO)₆₁(EO)₁₁₃OCH₃)₇; and polyethylenimine-graft-(—NH—CH₂CH(OH)CH₂O(PO)₆₁(EO)₁₁₃OCH₃)₁₅.

108. A polymer of the formula PEG5k-block-(CysC₁₈)₁₀-block-(Lys)₄₅; or PEG5k-block-(CysC₁₈)₁₀-block-(Lys)₂₀.

109. A method for enhancing expression of a nucleic acid in a cell, comprising contacting said cell with said nucleic acid and a penetration enhancer, such that the expression of the nucleic acid is enhanced.

110. The method of claim 108, wherein said penetration enhancer is a non-ionic agent, a negatively charged ionic agent, a cationic agent, a zwitterionic agent, a lipid derivative, a per-fluorinated agent, a natural or synthetic product or mixtures thereof.

111. The method of claim 109, wherein said non-ionic agent is selected from the group consisting of n-hexyl-β-glucopyranoside, n-heptyl-β-glucopyranoside, n-octyl-β-glucopyranoside, n-dodecyl-β-glucopyranoside, n-octyl-α-glucopyranoside, phenyl-β-glucopyranoside, n-hexyl-β-(D-1-thioglucopyranosides), n-heptyl-β-(D-1-thioglucopyranosides), n-octyl-β-(D-1-thioglucopyranosides), n-dodecyl-β-(D-1-thioglucopyranosides), n-octyl-β-(D)-galactopyranosides, n-dodecyl-β-(D)-galactopyranosides, N-decanoyl-N-methyl-glucamine, N-octanoyl-N-methyl-glucamine, and mixtures thereof.

112. The method of claim 108, where said negatively charged ionic agent is selected from the group consisting of: N-lauryl sarcosine salt, linolic acid salt, cholesteryl hydrogen succinate, DSPE-PEG, bile acid, hydrotropes, and mixtures thereof.

113. The method of claim 111, wherein said bile acid is selected from the group consisting of lithocholate, deoxycholate, glycodeoxycholate, taurodeoxycholate, chenodeoxycholate, glycochenodeoxycholate, taurochenodeoxycholate, ursodeoxycholate, glycoursodeoxycholate, tauroursodeoxycholate, cholate, glycocholate, taurocholate, ursocholate, glycoursocholate, tauroursocholate, and combinations thereof.

114. The method of claim 112, wherein said bile acid is a cholanic or a chenodeoxycholanic acid derivative.

115. The method of claim 112, wherein said penetration enhancer is selected from the group consisting of N¹-(eholic acid amide)-4,7,10-trioxo-1,13-tridecanediamine, N¹-(chenodeoxycholic acid amide)-4,7,10-trioxo-1,13-tridecanediamine; N-chenodeoxycholyl-2-aminoethyl-phosphonic acid monopotassium salt, and combinations thereof.

116. The method of claim 108, wherein said lipid membrane is a nuclear membrane.
